(19) United States
(12) Patent Application Publication
(10) Pub. No.: US 2023/0190831 A1
(43) Pub. Date: Jun. 22, 2023

(54) SOLID DOSAGE FORMS WITH IMPROVED DISINTEGRATION PROFILES

(71) Applicant: Evelo Biosciences, Inc., Cambridge, MA (US)

(72) Inventors: Syed Altaf, Cambridge, MA (US); Mike Frodsham, Flintshire (GB); James Graves, Flintshire (GB); Jiannan Lu, Cambridge, MA (US); Lance Pryce, Flintshire (GB)

(21) Appl. No.: 17/996,118

(22) PCT Filed: Apr. 16, 2021
(86) PCT No.: PCT/US2021/027731

§ 371 Date: Oct. 13, 2022

Related U.S. Application Data
(60) Provisional application No. 63/011,541, filed on Apr. 17, 2020.

(51) Int. Cl. A61K 35/745; A61K 35/744; A61K 35/741; A61K 9/20; A61K 9/48

(52) U.S. Cl. CPC A61K 35/745; A61K 9/2013; A61K 9/2018; A61K 9/2027; A61K 9/2054; A61K 9/2095; A61K 9/4808; A61K 35/741; A61K 35/744

(57) ABSTRACT

Methods and compositions related to improved solid dosage forms (e.g., minitablets) that facilitate the oral delivery of bacteria or agents of bacterial origin are provided herein.

### RELATED APPLICATIONS

This application claims the benefit of priority to U.S. Provisional Pat. Application having serial number 63/011,541, filed Apr. 17, 2020, the entire contents of which are hereby incorporated by reference in their entirety.

### BACKGROUND

The formulation of the solid dosage form of a pharmaceutical product can have a significant impact on the bioavailability of its active pharmaceutical ingredients. To improve bioavailability, a disintegration agent can be included in the solid dosage form. However, there are many potential disintegration agents available to choose from, each possessing its own properties. As the solid formulation disintegration process is complex and not well-understood, the effectiveness of any particular disintegration agent to facilitate the disintegration of a specific solid dose formulation is unpredictable. As a consequence, even with the addition of a disintegration agent, the disintegration rate of many solid dosage forms of pharmaceutical products can remain slow, adversely affecting active ingredient bioavailability.

### SUMMARY

This disclosure is based, in part, on the discovery of certain improved solid dosage forms that facilitate the oral delivery of bacteria and agents (e.g., components) of bacterial origin (e.g., microbial extracellular vesicles, or mEVs). For example, in certain embodiments the solid dosage forms disclosed herein include certain combinations and/or amounts of disintegration agents, resulting in a decrease in the disintegration time of the composition (e.g., 2-fold, 3-fold, 4-fold, 5-fold, 6-fold, 7-fold, 8-fold, 9-fold) as compared to conventional solid dosage forms (e.g., solid dosage forms containing conventional amounts of disintegration agents). In certain embodiments, the solid dosage forms provided herein result in an increase in therapeutic efficacy and/or physiological effect as compared to a pharmaceutical product having conventional solid dosage forms.

In certain aspects provided herein are solid dosage forms of pharmaceutical compositions. In certain embodiments, the solid dosage form comprises a pharmaceutical agent (e.g., bacteria and/or an agent of bacterial origin, such as mEVs, a powder comprising bacteria and/or an agent of bacterial origin, such as mEVs) and one or more disintegration agents (e.g., one, two or three disintegration agents). In certain embodiments, the solid dosage form comprises a pharmaceutical agent (e.g., bacteria and/or an agent of bacterial origin, such as mEVs, a powder comprising bacteria and/or an agent of bacterial origin, such as mEVs) and three disintegration agents. In certain embodiments, the total pharmaceutical agent mass is at least 0.5%, 1%, 5%, 10%, 20%, 25%, 30%, 40%, 50%, 60%, or 70% of the total mass of the pharmaceutical composition. In some embodiments, the total pharmaceutical agent mass is no more than 85%, 80%, 75%, 70%, or 50% of the total mass of the pharmaceutical composition. In some embodiments, the total mass of the one or more disintegrating agents is at least 5%, at least, 6%, at least 7%, at least 8%, at least 9%, at least 10%, at least 11%, or at least 12% of the total mass of the pharmaceutical composition. In some embodiments, the total mass of the one or more disintegrating agents is no more than 12%, 11%, 10%, 9%, or 8% of the total mass of the pharmaceutical composition. In some embodiments, the one or more disintegration agents comprise low-substituted hydroxypropyl cellulose (L-HPC, e.g., LH-11) and/or crospovidone (e.g., polyvinylpolypyrrolidone (PVPP), such as crospovidone CL-F)

In certain embodiments, the solid dosage forms provided herein comprise L-HPC, In some embodiments, the L-HPC is of grade LH-11. In certain embodiments, the total L-HPC mass is at least 0.1%, 0.5%, 1%, 2%, 3%, 4%, 5%, 6%, 7%, 8%, 9%, or 10% of the total mass of the pharmaceutical composition. In certain embodiments, the total L-HPC mass is no more than 0.1%, 0.5%, 1%, 2%, 3%, 4%, 5%, 6%, 7%, 8%, 9%, or 10% of the total mass of the pharmaceutical composition. In certain embodiments, the total L-HPC mass is about 0.1%, 0.5%, 1% 2%, 3%, 4%, 5%, 6%, 7%, 8%, 9%, or 10% of the total mass of the pharmaceutical composition. In certain embodiments, the total L-HPC (e.g., LH- 11, e.g., L-HPC LH-11) mass is about 0.5% of the total mass of the pharmaceutical composition. In certain embodiments, the total L-HPC (e.g., LH- 11) mass is about 5% of the total mass of the pharmaceutical composition.

In certain embodiments, the pharmaceutical composition does not comprise L-HPC. For example, a second pharmaceutical composition can comprise additional mannitol in an amount at which L-HPC had been present in a first pharmaceutical composition, e.g., a first pharmaceutical composition comprises about 36.5% mannitol and about 5% L-HPC, and a second pharmaceutical composition comprises about 41.5% mannitol and 0% L-HPC, wherein the amounts of the remaining components are the same in the first and second pharmaceutical compositions.

In certain embodiments, the solid dosage forms provided herein comprise crospovidone (e.g., polyvinylpolypyrrolidone (PVPP), such as crospovidone CL-F , such as Kollidon CL-F). In certain embodiments, the total crospovidone (e.g., PVPP) mass is at least 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., PVPP) mass is no more than 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., PVPP) mass is about 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., PVPP) mass is about 4% to about 10% of the total mass of the pharmaceutical composition. In certain embodiments, the total crospovidone (e.g., PVPP) mass is about 7% of the total mass of the pharmaceutical composition.

In certain embodiments, the solid dosage forms provided herein comprise: (i) a pharmaceutical agent having a total pharmaceutical agent mass that is at least 0.5% and no more than 75% of the total mass of the pharmaceutical composition, (ii) L-HPC (e.g., L-HPC of grade LH-11) having a total L-HPC mass that is at least 0.1% (e.g., at least 0.1%, 0.5%, 1%, 2%, 3%, 4%, 5%, 6%, 7%, 8%, 9%, or 10%) and no more than 10% (e.g., no more than 0.1%, 0.5%, 1%, 2%, 3%, 4%, 5%, 6%, 7%, 8%, 9%, or 10%) of the total mass of the pharmaceutical composition;; and (iii) crospovidone (e.g., polyvinylpolypyrrolidone (PVPP), such as crospovidone CL-F) having a total crospovidone (e.g., PVPP) mass that is at least 1% (e.g., at least 1%, 2%, 3%, 4%, 5%, 6%, 7%, 8%, 9%, 10%, 11%, 12%, 13%, 14%, or 15%) and no more than 15% (no more than 1%, 2%, 3%, 4%, 5%, 6%, 7%, 8%, 9%, 10%, 11%, 12%, 13%, 14%, or 15%) of the total mass of the pharmaceutical composition. In certain embodiments, the total L-HPC mass plus the total crospovidone (e.g., PVPP) mass is at least 5%, 6%, 7%, 8%, 9%, or 10% of the total mass of the pharmaceutical composition. In some embodiments, the solid dosage form comprises: a total L-HPC mass is about 0.5% of the total mass of the pharmaceutical composition; and a total crospovidone (e.g., PVPP) mass is about 7% of the total mass of the pharmaceutical composition. In some embodiments, the solid dosage form comprises: a total L-HPC mass is about 5% of the total mass of the pharmaceutical composition; and a total crospovidone (e.g., PVPP) mass is about 7% of the total mass of the pharmaceutical composition.

In certain embodiments, the solid dosage forms provided herein further comprise mannitol. In some embodiments, the mannitol is mannitol SD200. In certain embodiments, the total mannitol mass is at least 25%, 30%, 35%, 40%, 45%, 50%, 55%, 60%, 65%, 70%, or 75% of the total mass of the pharmaceutical composition. In certain embodiments, the total mannitol mass is no more than 25%, 30%, 35%, 40%, 45%, 50%, 55%, 60%, 65%, 70%, 75%, 80%, 85%, 90%, or 95% of the total mass of the pharmaceutical composition. In certain embodiments, the total mannitol mass is about 25%, 30%, 35%, 40%, 45%, 50%, 55%, 60%, 65%, 70%, 75%, 80%, 85%, , 90%, or 95% of the total mass of the pharmaceutical composition. In certain embodiments, the total mannitol (e.g., mannitol SD200) mass is about 26% to about 85% of the total mass of the pharmaceutical composition. In certain embodiments, the total mannitol (e.g., mannitol SD200) mass is about 26.5% of the total mass of the pharmaceutical composition. In certain embodiments, the total mannitol (e.g., mannitol SD200) mass is about 36.5% of the total mass of the pharmaceutical composition. In certain embodiments, the total mannitol (e.g., mannitol SD200) mass is about 56.5% of the total mass of the pharmaceutical composition. In certain embodiments, the total mannitol (e.g., mannitol SD200) mass is about 61% of the total mass of the pharmaceutical composition. In certain embodiments, the total mannitol (e.g., mannitol SD200) mass is about 70.5% of the total mass of the pharmaceutical composition. In certain embodiments, the total mannitol (e.g., mannitol SD200) mass is about 76% of the total mass of the pharmaceutical composition. In certain embodiments, the total mannitol (e.g., mannitol SD200) mass is about 80.5% of the total mass of the pharmaceutical composition. In certain embodiments, the total mannitol (e.g., mannitol SD200) mass is about 81.5% of the total mass of the pharmaceutical composition. In certain embodiments, the total mannitol (e.g., mannitol SD200) mass is about 83% of the total mass of the pharmaceutical composition. In certain embodiments, the total mannitol (e.g., mannitol SD200) mass is about 84.9% of the total mass of the pharmaceutical composition.

In certain embodiments, the solid dosage forms provided herein comprise magnesium stearate. In certain embodiments, the total magnesium stearate mass is at least 0.01%, 0.1%, 1%, 2%, 3%, 4%, 5%, 6%, 7%, 8%, 9%, 10%, or 11% of the total mass of the pharmaceutical composition. In certain embodiments, the total magnesium stearate mass is no more than 0.01%, 0.1%, 1%, 2%, 3%, 4%, 5%, 6%, 7%, 8%, 9%, 10%, or 11% of the total mass of the pharmaceutical composition. In certain embodiments, the total magnesium stearate mass is about 0.01%, 0.1%, 0.5%, 1%, 1.5%, 2%, 2.5%, 3%, 3.5%, 4%, 4.5%, 5%, 5.5%, 6%, 6.5%,7%, 7.5%, 8%, 8.5%, 9%, 9.5%, 10%, or 11% of the total mass of the pharmaceutical composition. In certain embodiments, the total magnesium stearate mass is about 0.5% to about 1.5% of the total mass of the pharmaceutical composition. In certain embodiments, the total magnesium stearate mass is about 0.5% of the total mass of the pharmaceutical composition. In certain embodiments, the total magnesium stearate mass is about 1% of the total mass of the pharmaceutical composition. In certain embodiments, the total magnesium stearate mass is about 1.5% of the total mass of the pharmaceutical composition. In certain embodiments, the total magnesium stearate mass is about 2% of the total mass of the pharmaceutical composition.

In certain embodiments, the solid dosage forms provided herein comprise colloidal silica dioxide (also referred to as colloidal silicon dioxide or silicon dioxide). In some embodiments, the colloidal silica dioxide is Aerosil 200. In certain embodiments, the total colloidal silica dioxide mass is at least 0.01%, 0.1%, 1%, 2%, 3%, 4%, 5%, 6%, 7%, 8%, 9%, 10%, or 11% of the total mass of the pharmaceutical composition. In certain embodiments, the total colloidal silica dioxide mass is no more than 0.01%, 0.1%, 1%, 2%, 3%, 4%, 5%, 6%, 7%, 8%, 9%, 10%, or 11% of the total mass of the pharmaceutical composition. In certain embodiments, the total colloidal silica dioxide mass is about 0.01%, 0.1%, 0.5%, 1%, 2%, 3%, 4%, 5%, 6%, 7%, 8%, 9%, 10%, or 11% of the total mass of the pharmaceutical composition. In certain embodiments, the total colloidal silica dioxide mass is about 0.5% to about 5% of the total mass of the pharmaceutical composition. In certain embodiments, the total colloidal silica dioxide mass is about 0.5% of the total mass of the pharmaceutical composition. In certain embodiments, the total colloidal silica dioxide mass is about 1% of the total mass of the pharmaceutical composition. In certain embodiments, the total colloidal silica dioxide mass is about 5% of the total mass of the pharmaceutical composition.

In certain embodiments, in the solid dosage forms provided herein, pharmaceutical agent (e.g., a powder comprising bacteria and/or an agent of bacterial origin, such as mEVs) and mannitol (e.g., mannitol SD200) comprise about 70%, 75%, 80%, 85%, 90%, or 95% of the mass of the solid dosage form. In certain embodiments, in the solid dosage forms provided herein, pharmaceutical agent (e.g., a powder comprising bacteria and/or an agent of bacterial origin, such as mEVs) and mannitol (e.g., mannitol SD200) comprise about 86% of the solid dosage form.

In certain embodiments, in the solid dosage forms provided herein, pharmaceutical agent (e.g., a powder comprising bacteria and/or an agent of bacterial origin, such as mEVs) and mannitol (e.g., mannitol SD200) comprise about 75%, 80%, 85%, 90%, 95%, or 99% of the mass of the solid dosage form. In certain embodiments, in the solid dosage forms provided herein, pharmaceutical agent (e.g., a powder comprising bacteria and/or an agent of bacterial origin, such as mEVs) and mannitol (e.g., mannitol SD200) comprise about 91% of the solid dosage form.

In certain embodiments, the solid dosage forms provided herein comprise about 25% pharmaceutical agent (e.g., a powder comprising bacteria and/or an agent of bacterial origin, such as mEVs); about 61% mannitol (e.g., mannitol SD200); about 5% L-HPC (e.g., L-HPC LH-11); about 7% crospovidone (e.g., PVPP); about 1.5% magnesium stearate; and about 0.5% colloidal silica dioxide.

In certain embodiments, the solid dosage forms provided herein comprise about 5% pharmaceutical agent (e.g., a powder comprising bacteria and/or an agent of bacterial origin, such as mEVs); about 80.5% mannitol (e.g., mannitol SD200); about 5% L-HPC (e.g., L-HPC LH-11); about 7% crospovidone (e.g., PVPP); about 2% magnesium stearate, and about 0.5% colloidal silica dioxide.

In certain embodiments, the solid dosage forms provided herein comprise about 50% pharmaceutical agent (e.g., a powder comprising bacteria and/or an agent of bacterial origin, such as mEVs); about 36.5% mannitol (e.g., mannitol SD200); about 0.5% L-HPC (e.g., L-HPC LH-11); about 7% crospovidone (e.g., PVPP); about 1% magnesium stearate; and about 5% colloidal silica dioxide.

In certain embodiments, the solid dosage forms provided herein comprise about 30% pharmaceutical agent (e.g., a powder comprising bacteria and/or an agent of bacterial origin, such as mEVs); about 56.5% mannitol (e.g., mannitol SD200); about 0.5% L-HPC (e.g., L-HPC LH-11); about 7% crospovidone (e.g., PVPP); about 1% magnesium stearate; and about 5% colloidal silica dioxide.

In certain embodiments, the solid dosage forms provided herein comprise about 10% pharmaceutical agent (e.g., a powder comprising bacteria and/or an agent of bacterial origin, such as mEVs); about 76% mannitol (e.g., mannitol SD200); about 0.5% L-HPC (e.g., L-HPC LH-11); about 7% crospovidone (e.g., PVPP); about 1.5% magnesium stearate; and about 5% colloidal silica dioxide.

In certain embodiments, the solid dosage forms provided herein comprise about 16% pharmaceutical agent (e.g., a powder comprising bacteria and/or an agent of bacterial origin, such as mEVs); about 70.5% mannitol (e.g., mannitol SD200); about 0.5% L-HPC (e.g., L-HPC LH-11); about 7% crospovidone (e.g., PVPP); about 1% magnesium stearate; and about 5% colloidal silica dioxide.

In certain embodiments, the solid dosage forms provided herein comprise about 60% pharmaceutical agent (e.g., a powder comprising bacteria and/or an agent of bacterial origin, such as mEVs); about 26.5% mannitol (e.g., mannitol SD200); about 5% L-HPC (e.g., L-HPC LH-11); about 7% crospovidone (e.g., PVPP); about 0.5% magnesium stearate; and about 1% colloidal silica dioxide.

In certain embodiments, the solid dosage forms provided herein comprise about 50% pharmaceutical agent (e.g., a powder comprising bacteria and/or an agent of bacterial origin, such as mEVs); about 36.5% mannitol (e.g., mannitol SD200); about 5% L-HPC (e.g., L-HPC LH-11); about 7% crospovidone (e.g., PVPP); about 0.5% magnesium stearate; and about 1% colloidal silica dioxide.

In certain embodiments, the solid dosage forms provided herein comprise about 5% pharmaceutical agent (e.g., a powder comprising bacteria, and/or an agent of bacterial origin, such as mEVs); about 81.5% mannitol (e.g., mannitol SD200); about 50% L-HPC (e.g., L-HPC LH-11); about 7% crospovidone (e.g., PVPP); about 0.5% magnesium stearate; and about 1% colloidal silica dioxide.

In certain embodiments, the solid dosage forms provided herein comprise about 3% pharmaceutical agent (e.g., a powder comprising bacteria and/or an agent of bacterial origin, such as mEVs); about 83% mannitol (e.g., mannitol SD200); about 0.5% L-HPC (e.g., L-HPC LH-11); about 7% crospovidone (e.g., PVPP); about 1.5% magnesium stearate; and about 5% colloidal silica dioxide.

In certain embodiments, the solid dosage forms provided herein comprise about 1.6% pharmaceutical agent (e.g., a powder comprising bacteria and/or an agent of bacterial origin, such as mEVs); about 84.9% mannitol (e.g., mannitol SD200); about 0.5% L-HPC (e.g., L-HPC LH-11); about 7% crospovidone (e.g., PVPP); about 1% magnesium stearate; and about 5% colloidal silica dioxide.

In certain embodiments, the total pharmaceutical agent mass is at least 5% and no more than 25% of the total mass of the pharmaceutical composition; the total mannitol mass is at least 61% and no more than 80.5% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is at least 1.5% and no more than 2% of the total mass of the pharmaceutical composition; and the total colloidal silicon dioxide mass is about 0.5% of the total mass of the pharmaceutical composition.

In certain embodiments, the total pharmaceutical agent mass is at least 5% and no more than 60% of the total mass of the pharmaceutical composition; the total mannitol mass is at least 26.5% and no more than 81.5% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

In certain embodiments, the total pharmaceutical agent mass is at least 3% and no more than 50% of the total mass of the pharmaceutical composition; the total mannitol mass is at least 36.5% and no more than 84.9% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

In certain embodiments, the total pharmaceutical agent mass is at least 10% and no more than 50% of the total mass of the pharmaceutical composition; the total mannitol mass is at least 56.5% and no more than 76% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

In certain embodiments, the total pharmaceutical agent mass is about 50% of the total mass of the pharmaceutical composition; the total mannitol mass is about 36.5% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1% of the total mass of the pharmaceutical composition; and the total colloidal silicon dioxide mass is about 0.5% of the total mass of the pharmaceutical composition.

In certain embodiments, the total pharmaceutical agent mass is at least 5% and no more than 60% of the total mass of the pharmaceutical composition; the total mannitol mass is at least 26% and no more than 81 % of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1.5% of the total mass of the pharmaceutical composition; and the total colloidal silicon dioxide mass is about 0.5% of the total mass of the pharmaceutical composition.

In certain embodiments, the total pharmaceutical agent mass is about 0.5% of the total mass of the pharmaceutical composition; the total mannitol mass is about 90.5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

In certain embodiments, the total pharmaceutical agent mass is about 5% of the total mass of the pharmaceutical composition; the total mannitol mass is about 86% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

In certain embodiments, the total pharmaceutical agent mass is about 25% of the total mass of the pharmaceutical composition; the total mannitol mass is about 66% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1 % of the total mass of the pharmaceutical composition; and the total colloidal silicon dioxide mass is about 1% of the total mass of the pharmaceutical composition.

In certain embodiments, the solid dosage forms of a pharmaceutical agent as described herein include minitablets. In some embodiments, the solid dosage form is enterically coated (e.g., comprises an enteric coating; e.g., is coated with an enteric coating). The minitablets are coated with one layer of enteric coating or with two layers of enteric coatings (e.g., an inner enteric coating and an outer enteric coating). The enterically-coated minitablets (with one layer of enteric coating or with two layers of enteric coatings (e.g., an inner enteric coating and an outer enteric coating)) can be loaded into a capsule; e.g., the capsule is not enterically coated.

In some embodiments, the solid dosage form comprises a minitablet. In some embodiments, the minitablet (e.g., enterically coated minitablet) is a 1 mm minitablet, 1.5 mm minitablet, 2 mm minitablet, 3 mm minitablet, or 4 mm minitablet. In some embodiments, a plurality of enterically coated minitablets are contained in a capsule (e.g., a size 0 capsule can contain about 31 to about 35 (e.g., 33) minitablets, wherein the minitablets are 3 mm in size). In some embodiments, the capsule is a size 00, size 0, size 1, size 2, size 3, size 4, or size 5 capsule. In some embodiments, the capsule comprises HPMC (hydroxyl propyl methyl cellulose) or gelatin.

In some embodiments, the enteric coating comprises one enteric coating.

In some embodiments, the enteric coating comprises an inner enteric coating and an outer enteric coating. In some embodiments, the enteric coating comprises an inner enteric coating and an outer enteric coating, and wherein the inner and outer enteric coatings are not identical (e.g., the inner and outer enteric coatings do not contain identical components in identical amounts).

In some embodiments, the enteric coating (e.g., the one enteric coating or the inner enteric coating and/or the outer enteric coating) comprises a polymethacrylate-based copolymer.

In some embodiments, the enteric coating (e.g., the one enteric coating or the inner enteric coating and/or the outer enteric coating) comprises a methacrylic acid ethyl acrylate (MAE) copolymer (1.1).

In some embodiments, the one enteric coating comprises methacrylic acid ethyl acrylate (MAE) copolymer (1:1) (such as Kollicoat MAE 100P).

In some embodiments, the one enteric coating comprises a Eudragit copolymer, e.g., a Eudragit L (e.g., Eudragit L 100-55; Eudragit L 30 D-5.5), a Eudragit S, a Eudragit RL, a Eudragit RS, a Eudragit E, or a Eudragit FS (e.g., Eudragit FS 30 D).

In some embodiments, the enteric coating (e.g., the one enteric coating or the inner enteric coating and/or the outer enteric coating) comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

In some embodiments, the enteric coating (e.g., the one enteric coating or the inner enteric coating and/or the outer enteric coating) comprises an anionic polymeric material.

The pharmaceutical agent can be of bacterial origin (e.g., mixture of selected strains or agents (e.g., components) thereof, such as microbial extracellular vesicles (mEVs) of the mixture of selected strains). The pharmaceutical agent can be of bacterial origin (e.g., a single selected strain and/or agents (e.g., components) thereof, such as microbial extracellular vesicles (mEVs) of that single selected strain). The pharmaceutical agent can be a powder that comprises the bacteria and/or components thereof, and, can comprise additional agents such as, e.g., cryoprotectant. For example, in some embodiments, the pharmaceutical agent is a lyophilized powder of bacteria and/or components thereof (e.g., mEVs) that optionally, further comprise additional agents, such as a cryoprotectant.

In some embodiments, the pharmaceutical agent comprises bacteria.

In some embodiments, the pharmaceutical agent comprises microbial extracellular vesicles (mEV).

In some embodiments, the pharmaceutical agent comprises bacteria and microbial extracellular vesicles (mEV).

In some embodiments, the pharmaceutical agent has one or more beneficial immune effects outside the gastrointestinal tract, e.g., when the solid dosage form is orally administered.

In some embodiments, the pharmaceutical agent modulates immune effects outside the gastrointestinal tract in the subject, e.g., when the solid dosage form is orally administered.

In some embodiments, the pharmaceutical agent causes a systemic effect (e.g., an effect outside of the gastrointestinal tract), e.g., when the solid dosage form is orally administered.

In some embodiments, the pharmaceutical agent acts on immune cells and/or epithelial cells in the small intestine (e.g., causing a systemic effect (e.g., an effect outside of the gastrointestinal tract), e.g., when the solid dosage form is orally administered.

In some embodiments, the pharmaceutical agent comprises isolated bacteria (e.g., from one or more strains of bacteria (e.g., bacteria of interest) (e.g., a therapeutically effective amount thereof)). E.g., wherein at least 5%, at least 10%, at least 25%, at least 50%, at least 75%, at least 80%, at least 85%, at least 90%, at least 95%, or at least 99% of the content of the pharmaceutical agent is the isolated bacteria (e.g., bacteria of interest).

In some embodiments, the pharmaceutical agent comprises bacteria that have been gamma irradiated, UV irradiated, heat inactivated, acid treated, or oxygen sparged.

In some embodiments, the pharmaceutical agent comprises live bacteria.

In some embodiments, the pharmaceutical agent comprises dead bacteria.

In some embodiments, the pharmaceutical agent comprises non-replicating bacteria.

In some embodiments, the pharmaceutical agent comprises bacteria from one strain of bacteria.

In some embodiments, the bacteria are lyophilized (e.g., the lyophilized product further comprises a pharmaceutically acceptable excipient) (e.g., a powder form).

In some embodiments, the bacteria are gamma irradiated.

In some embodiments, the bacteria are UV irradiated.

In some embodiments, the bacteria are heat inactivated (e.g., at 50° C. for two hours or at 90° C. for two hours).

In some embodiments, the bacteria are acid treated.

In some embodiments, the bacteria are oxygen sparged (e.g., at 0.1 vvm for two hours).

In some embodiments, the bacteria are Gram positive bacteria.

In some embodiments, the bacteria are Gram negative bacteria.

In some embodiments, the bacteria are aerobic bacteria.

In some embodiments, the bacteria are anaerobic bacteria. In some embodiments, the anaerobic bacteria comprise obligate anaerobes. In some embodiments, the anaerobic bacteria comprise facultative anaerobes. In some embodiments, the bacteria are acidophile bacteria.

In some embodiments, the bacteria are alkaliphile bacteria.

In some embodiments, the bacteria are neutralophile bacteria.

In some embodiments, the bacteria are fastidious bacteria.

In some embodiments, the bacteria are nonfastidious bacteria.

In some embodiments, the bacteria are of a taxonomic group (e.g., class, order, family, genus, species or strain) listed in Table 1, Table 2, or Table 3.

In some embodiments, the bacteria are a bacterial strain listed in Table 1, Table 2, or Table 3.

In some embodiments, the bacteria are of a taxonomic group (e.g., class, order, family, genus, species or strain) listed in Table J.

In some embodiments, the bacteria are a bacterial strain listed in Table J.

In some embodiments, the Gram negative bacteria belong to class Negativicutes.

In some embodiments, the Gram negative bacteria belong to family Veillonellaceae, Selenomonadaceae, Acidaminococcaceae, or Sporomusaceae.

In some embodiments, the bacteria of the genus Megasphaera, Selenomonas, Propionospora, or Acidaminococcus.

In some embodiments, the bacteria are Megasphaera sp., Selenomonas felix, Acidaminococcus intestine, or Propionospora sp. bacteria.

In some embodiments, the bacteria are of the genus Lactococcus, Prevotella, Bifidobacterium, or Veillonella.

In some embodiments, the bacteria are Lactococcus lactis cremoris bacteria.

In some embodiments, the bacteria are Prevotella histicola bacteria.

In some embodiments, the bacteria are Bifidobacterium animalis bacteria.

In some embodiments, the bacteria are Veillonellaparvula bacteria,

In some embodiments, the bacteria are Lactococcus lactis cremoris bacteria. In some embodiments, the Lactococcus factis cremoris bacteria are a strain comprising at least 90% (or at least 97%) genomic, 16S and/or CRISPR sequence identity to the nucleotide sequence of the Lactococcus lactis cremoris Strain A (ATCC designation number PTA-125368). In some embodiments, the Lactococcus bacteria are a strain comprising at least 99% genomic, 16S and/or CRISPR sequence identity to the nucleotide sequence of the Lactococcus factis cremoris Strain A (ATCC designation number PTA-125368). In some embodiments, the Lactococcus bacteria are Lactococcus factis cremoris Strain A (ATCC designation number PTA-125368).

In some embodiments, the bacteria are Prevotella bacteria. In some embodiments, the Prevotella bacteria are a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella bacteria are a strain comprising at least 99% genomic, 16S and/or CRISPR sequence identity to the nucleotide sequence of the Prevotella Strain B 50329 (NRRL accession number B 50329). In some embodiments, the Prevotella bacteria are Prevotella Strain B 50329 (NRRL accession number B 50329).

In some embodiments, the bacteria are Bifidobacterium bacteria. In some embodiments, the Bifidobacterium bacteria are from a strain comprising at least 90% (or at least 97%) genomic, 16S and/or CRISPR sequence identity to the nucleotide sequence of the Bifidobacterium bacteria deposited as ATCC designation number PTA-125097. In some embodiments, the Bifidobacterium bacteria are a strain comprising at least 99% genomic, 16S and/or CRISPR sequence identity to the nucleotide sequence of the Bifidobacterium bacteria deposited as ATCC designation number PTA-125097. In some embodiments, the Bifidobacterium bacteria are Bifidobacterium bacteria deposited as ATCC designation number PTA-125097.

In some embodiments, the bacteria are Veillonella bacteria. In some embodiments, the Veillonella bacteria are a strain comprising at least 90% (or at least 97%) genomic, 16S and/or CRISPR sequence identity to the nucleotide sequence of the Veillonella bacteria deposited as ATCC designation number PTA-125691. In some embodiments, the Veillonella. bacteria are a strain comprising at least 99% genomic, 16S and/or CRISPR sequence identity to the nucleotide sequence of the Veillonella bacteria deposited as ATCC designation number PTA-125691. In some embodiments, the Veillonella bacteria are Veillonella bacteria deposited as ATCC designation number PTA-125691.

In some embodiments, the bacteria are from Ruminococcus gnavus bacteria. In some embodiments, the Ruminococcus gnavus bacteria are a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are Ruminococcus gnavus bacteria deposited as ATCC designation number PTA-126695.

In some embodiments, the bacteria are Megasphaera sp. bacteria. In some embodiments, the Megasphaera. sp. bacteria are a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are Megasphaera sp. bacteria deposited as ATCC designation number PTA-126770.

In some embodiments, the bacteria are Fournierella massiliensis bacteria. In some embodiments, the Foumierella massiliensis bacteria are a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Foumierella massiliensis bacteria are a strain comprising at least 99% genomic, 16S and/or CRISPR sequence identity to the nucleotide sequence of the Foumierella massiliensis bacteria deposited as ATCC designation number PTA-126696. In some embodiments, the Fournierella massiliensis bacteria are Fournierella massiliensis bacteria deposited as ATCC designation number PTA-126696.

In some embodiments, the bacteria are Harryflintia acetispora bacteria. In some embodiments, the Harryflintia acetispora bacteria are a strain comprising at least 90% (or at least 97%) genomic, 16S and/or CRISPR sequence identity to the nucleotide sequence of the Harryflintia acetispora. bacteria deposited as ATCC designation number PTA-126694. In some embodiments, the Harryflintia acetispora bacteria are a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora. bacteria are Harryflintia acetispora bacteria deposited as ATCC designation number PTA-126694.

In some embodiments, the bacteria are of the family Acidaminococcaceae, Alcaligenaceae, Akkermansiaceae, Bacteriodaceae, Bifidobacteriaceae, Burkholderiaceae, Catabacteriaceae, Clostridiaceae, Coriobacteriaceae, Enterobacteriaceae, Enterococcaceae, Fusobacteriaceae, Lachnospiraceae, Listeraceae, Mycobacteriaceae, Neisseriaceae, Odoribacteraceae, Oscillospiraceae, Peptococcaceae, Peptostreptococcaceae, Porphyromonadaceae, Prevotellaceae, Propionibacteraceae, Rikenellaceae, Ruminococcaceae, Selenomonadaceae, Sporomusaceae, Streptococcaceae, Streptomycetaceae, Sutterellaceae, Synergistaceae, or Veillonellaceae.

In some embodiments, the bacteria are of the genus Akkermansia, Christensenella, Blautia, Enterococcus, Eubacterium, Roseburia, Bacteroides, Parabacteroides, or Erysipelatoclostridium.

In some embodiments, the bacteria are Blautia hydrogenotrophica, Blautia stercons, Blautia wexlerae, Eubacterium faecium, Eubacterium contortum, Eubacterium rectale, Enterococcus faecalis, Enterococcus durans, Enterococcus villorum, Enterococcus gallinarum; Bifidobacterium lactis, Bifidobacterium bifidium, Bifidobacterium longum, Bifidobacterium animalis, or Bifidobacterium breve bacteria.

In some embodiments, the bacteria are BCG (bacillus Calmette-Guerin), Parabacteroides, Blautia, Veillonella, Lactobacillus salivarius, Agathobaculum, Ruminococcus gnavus, Paraclostridium benzoelyticum, Turicibacter sanguinus, Burkholderia, Klebsiella quasipneumoniae ssp similpneumoniae, Klebsiella oxytoca, Tyzzerela nexilis, or Neisseria bacteria.

In some embodiments, the bacteria are Blautia hydrogenotrophica bacteria.

In some embodiments, the bacteria are Blautia stercoris bacteria

In some embodiments, the bacteria are Blautia wexlerae bacteria.

In some embodiments, the bacteria are Enterococcus gallinarum bacteria.

In some embodiments, the bacteria are Enterococcus faecium bacteria.

In some embodiments, the bacteria are Bifidobacterium bifidium bacteria.

In some embodiments, the bacteria are Bifidobacterium breve bacteria.

In some embodiments, the bacteria are Bifidobacterium longum bacteria.

In some embodiments, the bacteria are Roseburia hominis bacteria.

In some embodiments, the bacteria are Bacteroides thetaiotaomicron bacteria.

In some embodiments, the bacteria are Bacteroides coprocola bacteria.

In some embodiments, the bacteria are Erysipelatoclostridium ramosum bacteria.

In some embodiments, the bacteria are Megasphera massiliensis bacteria.

In some embodiments, the bacteria are Eubacterium bacteria.

In some embodiments, the bacteria are Parabacteroides distasonis bacteria.

In some embodiments, the bacteria are Lactobacillus plantarum bacteria.

In some embodiments, the bacteria are bacteria of the Negativicutes class.

In some embodiments, the bacteria are of the Veillonellaceae family.

In some embodiments, the bacteria are of the Selenomonadaceae family.

In some embodiments, the bacteria are of the Acidaminococcaceae family.

In some embodiments, the bacteria are of the Sporomusaceae family.

In some embodiments, the bacteria are of the Megasphaera genus.

In some embodiments, the bacteria are of the Selenomonas genus.

In some embodiments, the bacteria are of the Propionospora genus.

In some embodiments, the bacteria are of the Acidaminococcus genus.

In some embodiments, the bacteria are Megasphaera sp. bacteria.

In some embodiments, the bacteria are Selenomonasfelix bacteria.

In some embodiments, the bacteria are Acidaminococcus intestini bacteria.

In some embodiments, the bacteria are Propionospora sp. bacteria.

In some embodiments, the bacteria are bacteria of the Clostridia class.

In some embodiments, the bacteria are of the Oscillospriraceae family.

In some embodiments, the bacteria are of the Faecalibacterium genus.

In some embodiments, the bacteria are of the Fournierella genus.

In some embodiments, the bacteria are of the Harryflintia genus.

In some embodiments, the bacteria are of the Agathobaculum genus.

In some embodiments, the bacteria are Faecalibacterium prausnitzii (e.g., Faecalibacterium prausnitzii Strain A) bacteria.

In some embodiments, the bacteria are Fournierella massiliensis (e.g., Fournierella massiliensis Strain A) bacteria.

In some embodiments, the bacteria are Harryflintia acetispora (e.g., Harryflintia acetispora Strain A) bacteria.

In some embodiments, the bacteria are Agathobaculum sp. (e.g., Agathobaculum sp. Strain A) bacteria.

In some embodiments, the bacteria are a strain of Agathobaculum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the agathobaculum sp. strain is the Agathobaculum sp. Strain A (ATCC Deposit Number PTA- 125892).

In some embodiments, the bacteria are of the class Bacteroidia [phylum Bacteroidota]. In some embodiments, the bacteria are of order Bacteroidales. In some embodiments, the bacteria are of the family Porphyromonoadaceae. In some embodiments, the bacteria are of the family Prevotellaceae. In some embodiments, the bacteria are of the class Bacteroidia wherein the cell envelope structure of the bacteria is diderm. In some embodiments, the bacteria are of the class Bacteroidia that stain Gram negative. In some embodiments, the bacteria are of the class Bacteroidia wherein the bacteria is diderm and the bacteria stain Gram negative.

In some embodiments, the bacteria are of the class Clostridia [phylum Firmicutes]. In some embodiments, the bacteria are of the order Eubacteriales. In some embodiments, the bacteria are of the family Oscillispiraceae. In some embodiments, the bacteria are of the family Lachnospiraceae. In some embodiments, the bacteria are of the family Peptostreptococcaceae, In some embodiments, the bacteria are of the family Clostridialesfamily XIII/Incertae sedis41. In some embodiments, the bacteria are of the class Clostridia wherein the cell envelope structure of the bacteria is monoderm. In some embodiments, the bacteria are of the class Clostridia that stain Gram negative. In some embodiments, the bacteria are of the class Clostridia that stain Gram positive. In some embodiments, the bacteria are of the class Clostridia wherein the cell envelope structure of the bacteria is monoderm and the bacteria stain Gram negative. In some embodiments, the bacteria are of the class Clostridia wherein the cell envelope structure of the bacteria is monoderm and the bacteria stain Gram positive.

In some embodiments, the bacteria are of the class Negativicutes [phylum Firmicutes]. In some embodiments, the bacteria are of the order Veillonellales. In some embodiments, the bacteria are of the family Veillonelloceae. In some embodiments, the bacteria are of the order Selenomonadales. In some embodiments, the bacteria are of the family Selenomonadaceae. In some embodiments, the bacteria are of the family Sporomusaceae. In some embodiments, the bacteria are of the class Negativicutes wherein the cell envelope structure of the bacteria is diderm. In some embodiments, the bacteria are of the class Negativicutes that stain Gram negative. In some embodiments, the bacteria are of the class Negativicutes wherein the cell envelope structure of the bacteria is diderm and the bacteria stain Gram negative.

In some embodiments, the bacteria are of the class Synergistia [phylum Synergistota]. In some embodiments, the bacteria are of the order Synergistales. In some embodiments, the bacteria are of the family Synergistaceae. In some embodiments, the bacteria are of the class Synergistia wherein the cell envelope structure of the bacteria is diderm. In some embodiments, the bacteria are of the class Synergistia that stain Gram negative. In some embodiments, the bacteria are of the class Synergistia wherein the cell envelope structure of the bacteria is diderm and the bacteria stain Gram negative.

In some embodiments, the bacteria are bacteria that produce metabolites, e.g., the bacteria produce butyrate, iosine, proprionate, or tryptophan metabolites.

In some embodiments, the bacteria produce butyrate. In some embodiments, the bacteria are from the genus Blautia; Christensella; Copracoccus: Eubacterium; Lachnosperacea; Megasphaera; or Roseburia,

In some embodiments, the bacteria produce iosine. In some embodiments, the bacteria are from the genus Bifidobacterium; Lactobacillus; or Olsenella.

In some embodiments, the bacteria produce proprionate. In some embodiments, the bacteria are from the genus Akkermansia; Bacteriodes; Dialister; Eubacterium; Megasphaera; Parabacteriodes; Prevotella; Ruminococcus; or Veillonella.

In some embodiments, the bacteria produce tryptophan metabolites. In some embodiments, the bacteria are from the genus Lactobacillus or Peptostreptococcus.

In some embodiments, the bacteria are bacteria that produce inhibitors of histone deacetylase 3 (HDAC3). In some embodiments, the bacteria are from the species Bariatricus massiliensis, Faecalibacterium prausnitzii, Megasphaera massiliensis or Roseburia intestinalis.

In some embodiments, the pharmaceutical agent comprises isolated mEVs (e.g., from one or more strains of bacteria (e.g., bacteria of interest)) (e.g., a therapeutically effective amount thereof). E.g., wherein at 5%, at least 10%, at least 25%, at least 50%, at least 75%, at least 80%, at least 85%, at least 90%, at least 95%, or at least 99% of the content of the pharmaceutical agent is isolated mEV of bacteria (e.g., bacteria of interest).

In some embodiments, the pharmaceutical agent comprises mEVs and the mEVs comprise secreted mEVs (smEVs).

In some embodiments, the pharmaceutical agent comprises mEVs and the mEVs comprise processed mEVs (pmEVs).

In some embodiments, the pharmaceutical agent comprises pmEVs and the pmEVs are produced from bacteria that have been gamma irradiated, UV irradiated, heat inactivated, acid treated, or oxygen sparged.

In some embodiments, the pharmaceutical agent comprises pmEVs and the pmEVs are produced from live bacteria.

In some embodiments, the pharmaceutical agent comprises pmEVs and the pmEVs are produced from dead bacteria.

In some embodiments, the pharmaceutical agent comprises pmEVs and the pmEVs are produced from non-replicating bacteria.

In some embodiments, the pharmaceutical agent comprises mEVs and the mEVs are from one strain of bacteria.

In some embodiments, the mEVs are lyophilized (e.g., the lyophilized product further comprises a pharmaceutically acceptable excipient).

In some embodiments, the mEVs are gamma irradiated.

In some embodiments, the mEVs are UV irradiated.

In some embodiments, the mEVs are heat inactivated (e.g., at 50° C. for two hours or at 90° C. for two hours).

In some embodiments, the mEVs are acid treated.

In some embodiments, the mEVs are oxygen sparged (e.g., at 0.1 vvm for two hours).

In some embodiments, the mEVs are from Gram positive bacteria.

In some embodiments, the mEVs are from Gram negative bacteria.

In some embodiments, the mEVs are from aerobic bacteria.

In some embodiments, the mEVs are from anaerobic bacteria. In some embodiments, the anaerobic bacteria comprise obligate anaerobes. In some embodiments, the anaerobic bacteria comprise facultative anaerobes.

In some embodiments, the mEVs are from acidophile bacteria.

In some embodiments, the mEVs are from alkaliphile bacteria.

In some embodiments, the mEVs are from neutralophile bacteria.

In some embodiments, the mEVs are from fastidious bacteria.

In some embodiments, the mEVs are from nonfastidious bacteria.

In some embodiments, the mEVs are from bacteria of a taxonomic group (e.g., class, order, family, genus, species or strain) listed in Table 1, Table 2, or Table 3.

In some embodiments, the mEVs are from a bacterial strain listed in Table 1, Table 2, or Table 3.

In some embodiments, the mEVs are from bacteria of a taxonomic group (e.g., class, order, family, genus, species or strain) listed in Table J.

In some embodiments, the mEVs are from a bacterial strain listed in Table J.

In some embodiments, the Gram negative bacteria belong to class Negativicutes.

In some embodiments, the Gram negative bacteria belong to family Veillonellaceae, Selenomonadaceae, Acidaminococcaceae, or Sporomusaceae.

In some embodiments, the mEVs are from bacteria of the genus Megasphaera, Selenomonas, Propionospora, or Acidaminococcus.

In some embodiments, the mEVs are Megasphaera sp., Selenomonas felix, Acidaminococcus intestine, or Propionospora sp. bacteria,

In some embodiments, the mEVs are from bacteria of the genus Lactococcus, Prevotella, Bifidobacterium, or Veillonella.

In some embodiments, the mEVs are from Lactococcus lactis cremoris bacteria.

In some embodiments, the mEVs are from Prevotella histicola bacteria.

In some embodiments, the mEVs are from Bifidobacterium animalis bacteria.

In some embodiments, the mEVs are from Veillonella parvula bacteria.

In some embodiments, the mEVs are from Lactococcus lactis cremoris bacteria. In some embodiments, the Lactococcus lactis cremoris bacteria are from a strain comprising at least 90% (or at least 97%) genomic, 16S and/or CRISPR sequence identity to the nucleotide sequence of the Lactococcus lactis cremoris Strain A (ATCC designation number PTA-125368). In some embodiments, the Lactococcus bacteria are from a strain comprising at least 99% genomic, 16S and/or CRISPR sequence identity to the nucleotide sequence of the Lactococcus lactis cremoris Strain A (ATCC designation number PTA-125368). In some embodiments, the Lactococcus bacteria are from Lactococcus lactis cremoris Strain A (ATCC designation number PTA-125368).

In some embodiments, the mEVs are from Prevotella bacteria. In some embodiments, the Prevotella bacteria are from a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella bacteria are from a strain comprising at least 99% genomic, 16S and/or CRISPR sequence identity to the nucleotide sequence of the Prevotella Strain B 50329 (NRRL accession number B 50329). In some embodiments, the Prevotella bacteria are from Prevotella Strain B 50329 (NRRL accession number B 50329).

In some embodiments, the mEVs are from Bifidobacterium bacteria. In some embodiments, the Bifidobacterium bacteria are from a strain comprising at least 90% (or at least 97%) genomic, 16S and/or CRISPR sequence identity to the nucleotide sequence of the Bifidobacterium bacteria deposited as ATCC designation number PTA-125097. In some embodiments, the Bifidobacterium bacteria are from a strain comprising at least 99% genomic, 16S and/or CRISPR sequence identity to the nucleotide sequence of the Bifidobacterium bacteria deposited as ATCC designation number PTA-125097. In some embodiments, the Bifidobacterium bacteria are from Bifidobacterium bacteria deposited as ATCC designation number PTA-125097.

In some embodiments, the mEVs are from Veillonella bacteria. In some embodiments, the Veillonella bacteria are from a strain comprising at least 90% (or at least 97%) genomic, 16S and/or CRISPR sequence identity to the nucleotide sequence of the Veillonella bacteria deposited as ATCC designation number PTA-125691. In some embodiments, the Veillonella bacteria are from a strain comprising at least 99% genomic, 16S and/or CRISPR sequence identity to the nucleotide sequence of the Veillonella bacteria deposited as ATCC designation number PTA-125691. In some embodiments, the Veillonella bacteria are from Veillonella bacteria deposited as ATCC designation number PTA-125691.

In some embodiments, the mEVs are from Ruminococcus gnavus bacteria. In some embodiments, the Ruminococcus gnavus bacteria are from a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are from a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are from Ruminococcus gnavus bacteria deposited as ATCC designation number PTA-126695.

In some embodiments, the mEVs are from Megasphaera sp. bacteria. In some embodiments, the Megasphaera sp. bacteria are from a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are from a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are from Megasphaera sp. bacteria deposited as ATCC designation number PTA-126770.

In some embodiments, the mEVs are from Fournierella massiliensis bacteria. In some embodiments, the Fournierella massiliensis bacteria are from a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are from a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are from Fournierella massiliensis bacteria deposited as ATCC designation number PTA-126696.

In some embodiments, the mEVs are from Harryflintia acetispora bacteria. In some embodiments, the Harryflintia acetispora bacteria are from a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are from a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are from Harryflintia acetispora bacteria deposited as ATCC designation number PTA-126694.

In some embodiments, the mEVs are from bacteria of the family Acidaminococcaceae, Alcaligenaceae, Akkermansiaceae, Bacteriodaceae, Bifidobacteriaceae, Burkholderiaceae, Catabacteriaceae, Clostridiaceae, Coriobacteriaceae, Enterobacteriaceae, Enterococcaceae, Fusobacteriaceae, Lachnospiraceae, Listeraceae, Mycobacteriaceae, Neisseriaceae, Odoribacteraceae, Oscillospiraceae, Peptococcaceae, Peptostreptococcaceae, Porphyromonadaceae, Prevotellaceae, Propionibacteraceae, Rikenellaceae, Ruminococcaceae, Selenomonadaceae, Sporomusaceae, Streptococcaceae, Streptomycetaceae, Sutterellaceae, Synergistaceae, or Veillonellaceae.

In some embodiments, the mEVs are from bacteria of the genus Akkermansia, Christensonelia, Blautia, Enterococcus, Eubacterium, Roseburia, Bacteroides, Parabacteroides, or Erysipelatoclostridium.

In some embodiments, the mEVs are from Blautia hydrogenotrophica, Blautia stercoris, Blautia wexlerae, Eubacterium faecium, Eubacterium contortum, Eubacterium rectale, Enterococcus faecalis, Enterococcus durans, Enterococcus villorum, Enterococcus gallinarum; Bifidobacterium lactis, Bifidobacterium bifidium, Bifidobacterium longum, Bifidobacterium animalis, or Bifidobacterium breve bacteria.

In some embodiments, the mEVs are from BCG (bacillus Calmette-Guerin), Parabacteroides, Blautia, Veillonella, Lactobacillus salivarius, Agathobaculum, Ruminococcus gnavus, Paraclostridium benzoelyticum, Turicibacter sanguinus, Burkholderia, Klebsiella quasipneumoniae ssp similpneumoniae, Klebsiella oxytoca, Tyzzerela nexilis, or Neisseria bacteria.

In some embodiments, the mEVs are from Blautia hydrogenotrophica bacteria.

In some embodiments, the mEVs are from Blautia stercoris bacteria.

In some embodiments, the mEVs are from Blautia wexlerae bacteria.

In some embodiments, the mEVs are from Enterococcus gallinarum bacteria.

In some embodiments, the mEVs are from Enterococcus faecium bacteria.

In some embodiments, the mEVs are from Bifidobacterium bifidium bacteria.

In some embodiments, the mEVs are from Bifidobacterium breve bacteria.

In some embodiments, the mEVs are from Bifidobacterium longum bacteria.

In some embodiments, the mEVs are from Roseburia hominis bacteria.

In some embodiments, the mEVs are from Bacteroides thetaiotaomicron bacteria.

In some embodiments, the mEVs are from Bacteroides coprocola bacteria.

In some embodiments, the mEVs are from Erysipelatoclostridium ramosum bacteria.

In some embodiments, the mEVs are from Megasphera massiliensis bacteria.

In some embodiments, the mEVs are from Eubacterium bacteria.

In some embodiments, the mEVs are from Parabacteroides distasonis bacteria.

In some embodiments, the mEVs are from Lactobacillus plantarum bacteria.

In some embodiments, the mEVs are from bacteria of the Negativicutes class.

In some embodiments, the mEVs are from bacteria of the Veillonellaceae family.

In some embodiments, the mEVs are from bacteria of the Selenomonadaceae family.

In some embodiments, the mEVs are from bacteria of the Acidaminococcaceae family.

In some embodiments, the mEVs are from bacteria of the Sporomusaceae family.

In some embodiments, the mEVs are from bacteria of the Megasphaera genus.

In some embodiments, the mEVs are from bacteria of the Selenomonas genus.

In some embodiments, the mEVs are from bacteria of the Propionospora genus.

In some embodiments, the mEVs are from bacteria of the Acidaminococcus genus.

In some embodiments, the mEVs are from Megasphaera sp. bacteria.

In some embodiments, the mEVs are from Selenomonas felix bacteria.

In some embodiments, the mEVs are from Acidaminococcus intestini bacteria.

In some embodiments, the mEVs are from Propionospora sp. bacteria.

In some embodiments, the mEVs are from bacteria of the Clostridia class.

In some embodiments, the mEVs are from bacteria of the Oscillospriraceae family.

In some embodiments, the mEVs are from bacteria of the Faecalibacterium genus.

In some embodiments, the mEVs are from bacteria of the Fournierella genus.

In some embodiments, the mEVs are from bacteria of the Harryflintia genus.

In some embodiments, the mEVs are from bacteria of the Agathobaculum genus.

In some embodiments, the mEVs are from Faecalibacterium prausnitzii (e.g., Faecalibacterium prausnitzii Strain A) bacteria.

In some embodiments, the mEVs are from Fournierella massiliensis (e.g., Fournierella massiliensis Strain A) bacteria.

In some embodiments, the mEVs are from Harryflintia acetispora (e.g., Harryflintia acetispora Strain A) bacteria.

In some embodiments, the mEVs are from Agathobaculum sp. (e.g., Agathobaculum sp. Strain A) bacteria.

In some embodiments, the mEVs are from a strain of Agathobaculum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA- 125892).

In some embodiments, the mEVs are from bacteria of the class Bacteroidia [phylum Bacteroidota]. In some embodiments, the mEVs are from bacteria of order Bacteroidales. In some embodiments, the mEVs are from bacteria of the family Porphyromonoadaceae. In some embodiments, the mEVs are from bacteria of the family Prevotellaceae. In some embodiments, the mEVs are from bacteria of the class Bacteroidia wherein the cell envelope structure of the bacteria is diderm. In some embodiments, the mEVs are from bacteria of the class Bacteroidia that stain Gram negative. In some embodiments, the mEVs are from bacteria of the class Bacteroidia wherein the bacteria is diderm and the bacteria stain Gram negative.

In some embodiments, the mEVs are from bacteria of the class Clostridia [phylum Firmicutes]. In some embodiments, the mEVs are from bacteria of the order Eubacteriales. In some embodiments, the mEVs are from bacteria of the family Oscillispiraceae. In some embodiments, the mEVs are from bacteria of the family Lachnospiraceae. In some embodiments, the mEVs are from bacteria of the family Peptostreptococcaceae. In some embodiments, the mEVs are from bacteria of the family Clostridialesfamily XIII/Incertae sedis41. In some embodiments, the mEVs are from bacteria of the class Clostridia wherein the cell envelope structure of the bacteria is monoderm. In some embodiments, the mEVs are from bacteria of the class Clostridia that stain Gram negative. In some embodiments, the mEVs are from bacteria of the class Clostridia that stain Gram positive. In some embodiments, the mEVs are from bacteria of the class Clostridia wherein the cell envelope structure of the bacteria is monoderm and the bacteria stain Gram negative. In some embodiments, the mEVs are from bacteria of the class Clostridia wherein the cell envelope structure of the bacteria is monoderm and the bacteria stain Gram positive.

In some embodiments, the mEVs are from bacteria of the class Negativicutes [phylum Firmicutes]. In some embodiments, the mEVs are from bacteria of the order Veillonellales. In some embodiments, the mEVs are from bacteria of the family Veillonelloceae. In some embodiments, the mEVs are from bacteria of the order Selenomonadales. In some embodiments, the mEVs are from bacteria of the family Selenomonadaceae. In some embodiments, the mEVs are from bacteria of the family Sporomusaceae. In some embodiments, the mEVs are from bacteria of the class Negativicutes wherein the cell envelope structure of the bacteria is diderm. In some embodiments, the mEVs are from bacteria of the class Negativicutes that stain Gram negative. In some embodiments, the mEVs are from bacteria of the class Negativicutes wherein the cell envelope structure of the bacteria is diderm and the bacteria stain Gram negative.

In some embodiments, the mEVs are from bacteria of the class Synergistia [phylum Synergistota]. In some embodiments, the mEVs are from bacteria of the order Synergistales. In some embodiments, the mEVs are from bacteria of the family Synergistaceae. In some embodiments, the mEVs are from bacteria of the class Synergistia wherein the cell envelope structure of the bacteria is diderm. In some embodiments, the mEVs are from bacteria of the class Synergistia that stain Gram negative. In some embodiments, the mEVs are from bacteria of the class Synergistia wherein the cell envelope structure of the bacteria is diderm and the bacteria stain Gram negative.

In some embodiments, the mEVs are from bacteria that produce metabolites, e.g., the bacteria produce butyrate, iosine, proprionate, or tryptophan metabolites.

In some embodiments, the bacteria produce butyrate. In some embodiments, the bacteria are from the genus Blautia; Christensella; Copracoccus; Eubacterium; Lachnosperacea; Megasphaera; or Roseburia.

In some embodiments, the bacteria produce iosine. In some embodiments, the bacteria are from the genus Bifidobacterium; Lactobacillus; or Olsenella.

In some embodiments, the bacteria produce proprionate. In some embodiments, the bacteria are from the genus Akkermansia; Bacteriodes; Dialister; Eubacterium; Megasphaera; Parabacteriodes; Prevotella; Ruminococcus; or Veillonella.

In some embodiments, the bacteria produce tryptophan metabolites. In some embodiments, the bacteria are from the genus Lactobacillus or Peptostreptococcus.

In some embodiments, the mEVs are from bacteria that produce inhibitors of histone deacetylase 3 (HDAC3). In some embodiments, the bacteria are from the species Bariatricus massiliensis, Faecalibacterium prausnitzii, Megasphaera massiliensis or Roseburia intestinalis.

In some embodiments, the pharmaceutical agent comprises bacteria and the dose of bacteria is about 1 × 10⁷ to about 2 × 10¹² (e.g., about 3 × 10¹⁰ or about 1.5 × 10¹¹ or about 1.5 × 10¹²) cells (e.g., wherein cell number is determined by total cell count, which is determined by Coulter counter), wherein the dose is per capsule or tablet or per total number of minitablets in a capsule. In some embodiments, the pharmaceutical agent comprises bacteria and the dose of bacteria is about 1 × 10¹⁰ to about 2 × 10¹² (e.g., about 1.6 × 10¹¹ or about 8 × 10¹¹ or about 9.6 × 10¹¹ about 12.8 × 10¹¹ or about 1.6 × 10¹²) cells (e.g., wherein cell number is determined by total cell count, which is determined by Coulter counter), wherein the dose is per capsule or tablet or per total number of minitablets in a capsule.

In some embodiments, the pharmaceutical agent comprises bacteria and the dose of bacteria is about 1 × 10⁹, about 3 × 10⁹, about 5 × 10⁹, about 1.5 × 10¹⁰, about 3 × 10¹⁰, about 5 × 10¹⁰, about 1.5 × 10¹¹ about 1.5 × 10¹², or about 2 × 10¹² cells, wherein the dose is per capsule or tablet or per total number of minitablets in a capsule.

In some embodiments, the pharmaceutical agent comprises mEVs and the dose of mEVs is about 1 × 10⁵ to about 7 × 10¹³ particles (e.g., wherein particle count is determined by NTA (nanoparticle tracking analysis)), wherein the dose is per capsule or tablet or per total number of minitablets in a capsule. In some embodiments, the pharmaceutical agent comprises mEVs and the dose of mEVs is about 1 × 10¹⁰ to about 7 × 10¹³ particles (e.g., wherein particle count is determined by NTA (nanoparticle tracking analysis)), wherein the dose is per capsule or tablet or per total number of minitablets in a capsule.

In some embodiments, the pharmaceutical agent comprises a powder comprising bacteria and/or mEVs and the dose of the pharmaceutical agent (e.g., a powder comprising bacteria and/or mEVs) is about 10 mg to about 3500 mg, wherein the dose is per total number of minitablets in a capsule.

In some embodiments, the pharmaceutical agent comprises a powder comprising bacteria and/or mEVs and the dose of the pharmaceutical agent (e.g., a powder comprising bacteria and/or mEVs) is about 30 mg to about 1300 mg (by weight of bacteria and/or mEVs powder) (about 25, about 30, about 35, about 50, about 75, about 100, about 120, about 150, about 250, about 300, about 350, about 400, about 500, about 600, about 700, about 750, about 800, about 900, about 1000, about 1100, about 1200, about 1250, about 1300, about 2000, about 2500, about 3000, or about 3500 mg wherein the dose is per total number of minitablets in a capsule.

In some embodiments, the pharmaceutical agent comprises bacteria and/or mEVs and the dose of pharmaceutical agent (e.g., bacteria and/or mEVs) is about 2×10⁶ to about 2×10¹⁶ particles (e.g., wherein particle count is determined by NTA (nanoparticle tracking analysis)), wherein the dose is per total number of minitablets in a capsule.

In some embodiments, the pharmaceutical agent comprises bacteria and/or mEVs and the dose of pharmaceutical agent (e.g., bacteria and/or mEVs) is about 5 mg to about 900 mg total protein (e.g., wherein total protein is determined by Bradford assay or BCA), wherein the dose is per total number of minitablets in a capsule.

In some embodiments, the solid dosage form further comprises one or more additional therapeutic agents.

In some aspects, the disclosure provides a method of treating a subject (e.g., human) (e.g., a subject in need of treatment), the method comprising administering to the subject a solid dosage form provided herein. In some aspects, the disclosure provides use of a solid dosage form provided herein for the preparation of a medicament for treating a subject (e.g., human) (e.g., a subject in need of treatment).

In some embodiments, the solid dosage form is orally administered (e.g., is for oral administration).

In some embodiments, the solid dosage form is administered to a subject that is in a fed or fasting state. In some embodiments, the solid dosage form is administered to a subject on an empty stomach (e.g., one hour before eating or two hours after eating). In some embodiments, the solid dosage form is administered to a subject one hour before eating. In some embodiments, the solid dosage form is administered to a subject two hours after eating.

In some embodiments, the solid dosage form (e.g., plurality of minitablets (e.g., contained in a capsule)) is administered (e.g., is for administration) 1, 2, 3, or 4 times a day. In some embodiments, the solid dosage form comprises a plurality of minitablets (e.g., contained in a capsule) and 1, 2, 3, or 4 solid dosage forms (e.g., a plurality of minitablets (e.g., contained in a capsule)) are administered (e.g., are for administration) 1, 2, 3, or 4 times a day.

In some embodiments, the solid dosage form provides release of the pharmaceutical agent in the small intestine, e.g., in the upper small intestine, of the pharmaceutical agent contained in the solid dosage form.

In some embodiments, the solid dosage form delivers the pharmaceutical agent to the small intestine, wherein the pharmaceutical agent can act on immune cells and/or epithelial cells in the small intestine, e.g., in the upper small intestine, e.g., to cause effects throughout the body (e.g., systemic effect).

In some embodiments, the pharmaceutical agent provides one or more beneficial immune effects outside the gastrointestinal tract, e.g., when orally administered.

In some embodiments, the pharmaceutical agent modulates immune effects outside the gastrointestinal tract in the subject, e.g., when orally administered.

In some embodiments, the pharmaceutical agent causes a systemic effect (e.g., an effect outside of the gastrointestinal tract), e.g., when orally administered.

In some embodiments, the pharmaceutical agent acts on immune cells and/or epithelial cells in the small intestine (e.g., upper small intestine) (e.g., causing a systemic effect (e.g., an effect outside of the gastrointestinal tract), e.g., when orally administered.

In some embodiments, the solid dosage form is administered orally and has one or more beneficial immune effects outside the gastrointestinal tract (e.g., interaction between the agent and cells in the small intestine modulates a systemic immune response).

In some embodiments, the solid dosage form is administered orally and modulates immune effects outside the gastrointestinal tract (e.g., interaction between agent and cells in the small intestine (e.g., upper small intestine) modulates a systemic immune response).

In some embodiments, the solid dosage form is administered orally and activates innate antigen presenting cells (e.g., in the small intestine, e.g., upper small intestine).

In some embodiments, the subject is in need of treatment (and/or prevention) of a cancer.

In some embodiments, the subject is in need of treatment (and/or prevention) of an autoimmune disease.

In some embodiments, the subject is in need of treatment (and/or prevention) of an inflammatory disease.

In some embodiments, the subject is in need of treatment (and/or prevention) of a metabolic disease.

In some embodiments, the subject is in need of treatment (and/or prevention) of a dysbiosis.

In some embodiments, the solid dosage form is administered in combination with a therapeutic agent (e.g., additional therapeutic agent).

In certain aspects, provided herein are methods of preparing a solid dosage form of a pharmaceutical composition, the method comprising combining into a pharmaceutical composition a pharmaceutical agent (e.g., bacteria disclosed herein and/or an agent of bacterial origin, such as mEVs disclosed herein) and one or more (e.g., one, two or three) disintegration agents. In certain embodiments, the total pharmaceutical agent mass is at least 0.5%, 1%, 10%, 20%, 40%, 60%, or 70% of the total mass of the pharmaceutical composition. In some embodiments, the total pharmaceutical agent mass is no more than 85%, 80%, 75%, or 70% of the total mass of the pharmaceutical composition. In some embodiments, the total mass of the one or more disintegrating agents is at least 5%, at least, 6%, at least 7%, at least 8%, at least 9%, at least 10%, at least 11%, or at least 12%. In some embodiments, the total mass of the one or more disintegrating agents is no more than 12%, 11%, 10%, 9%, or 8% of the total mass of the pharmaceutical composition.

In some embodiments, the one or more disintegration agents comprise low-substituted hydroxypropyl cellulose (L-HPC) and/or crospovidone (e.g, PVPP, such as crospovidone CL-F). In certain embodiments, the solid dosage forms provided herein comprise L-HPC. In some embodiments, the L-HPC is of grade LH-11. In certain embodiments, the total L-HPC mass is at least 0.1%, 0.5%, 1%, 2%, 3%, 4%, 5%, 6%, 7%, 8%, 9%, or 10% of the total mass of the pharmaceutical composition. In certain embodiments, the total L-HPC mass is no more than 0.1%, 0.5%, 1%, 2%, 3%, 4%, 5%, 6%, 7%, 8%, 9%, or 10% of the total mass of the pharmaceutical composition. In certain embodiments, the total L-HPC mass is 0.1%, 0.5%, 1%, 2%, 3%, 4%, 5%, 6%, 7%, 8%, 9%, or 10% of the total mass of the pharmaceutical composition. In certain embodiments, the solid dosage forms provided herein comprise (e.g., polyvinylpolypyrrolidone (PVPP), such as crospovidone CL-F). In certain embodiments, the total (e.g., polyvinylpolypyrrolidone (PVPP), such as crospovidone CL-F)mass is at least 1%, 2%, 3%, 4%, 5%, 6%, 7%, 8%, 9%, 10%, 11%, 12%, 13%, 14%, or 15% of the total mass of the pharmaceutical composition. In certain embodiments, the total (e.g., polyvinylpolypyrrolidone (PVPP), such as crospovidone CL-F)mass is no more than 1%, 2%, 3%, 4%, 5%, 6%, 7%, 8%, 9%, 10%, 11%, 12%, 13%, 14%, or 15% of the total mass of the pharmaceutical composition. In certain embodiments, the total (e.g., polyvinylpolypyrrolidone (PVPP), such as crospovidone CL-F)mass is about 1%, 2%, 3%, 4%, 5%, 6%, 7%, 8%, 9%, 10%, 11%, 12%, 13%, 14%, or 15% of the total mass of the pharmaceutical composition.

In certain embodiments, the method further comprises compressing the pharmaceutical composition, thereby forming a minitablet. In some embodiments, the method further comprises enterically coating the minitablet. In certain embodiments, the method further comprises loading the minitablets into a capsule.

### BRIEF DESCRIPTION OF THE DRAWINGS

FIG. 1 is a graph showing 24-hour ear measurements in a DTH model with the treatments and doses listed, including uncoated solid dosage forms containing smEVs (labeled mEVs) from Prevotella histicola Strain B. Change in ear thickness (mm) was measured. MMT: mini-mini-tablet solid dosage form.

FIG. 2 is a graph showing 24-hour ear measurements in a DTH model with the treatments and doses listed, including uncoated solid dosage forms containing Prevotella histicola Strain B. Change in ear thickness (mm) was measured. MMT: mini-mini-tablet solid dosage form.

FIG. 3 is a graph showing 24-hour ear measurements in a DTH model with the treatments and doses listed, including uncoated solid dosage forms containing Veillonella parvula. Change in ear thickness (mm) was measured. MMT: mini-mini-tablet solid dosage form.

### DETAILED DESCRIPTION

As described herein, for a solid dosage form to contain a given amount (e.g., dose) of active ingredient (e.g., pharmaceutical agent, e.g, bacteria and/or an agent of bacterial origin, such as mEVs, a powder comprising bacteria and/or an agent of bacterial origin, such as mEVs), the amount of pharmaceutical agent (that contains the active ingredient) incorporated into a solid dosage form may be adjusted depending on the amount of active ingredient contained in a given preparation (e.g., batch) of pharmaceutical agent. The amount of diluent (such as mannitol) is then adjusted accordingly. For example, if the amount of pharmaceutical agent is increased, the amount of diluent is decreased; and vice versa. As described herein, adjustments can be made to the amounts of pharmaceutical agent and diluent, yet the amount of one or more excipients (e.g., one, two or three excipients) remains constant, e.g., batch to batch for a given solid dosage form recipe. Similarly, the amounts of magnesium stearate and colloidal silica can also remain constant, e.g., batch to batch for a given solid dosage form recipe.

For example, in the working examples provided herein, pharmaceutical agent containing Prevotella histicola powder was used to prepare three solid dosage forms (e.g., Formulation 2). The three preparations contained 1.5% magnesium stearate and 0.5% colloidal silica. Yet in the three preparations, the pharmaceutical agent was used at 25%, 60%, or 5%. To adjust for the differing amounts of pharmaceutical agent, the amount of mannitol was differed: 61% mannitol when 25% pharmaceutical agent was used; 26% mannitol when 60% pharmaceutical agent was used; 81%) mannitol when 5% pharmaceutical agent was used. In each of these preparations, the low-substituted hydroxypropyl cellulose was used at 5%; and the crospovidone was used at 7%.

As another example, in the working examples provided herein, pharmaceutical agent containing powder of smEVs from Prevotella histicola was used to prepare three solid dosage forms. The three preparations contained 1% magnesium stearate and 1% colloidal silica. Yet in the three preparations, the pharmaceutical agent was used at 25%, 5%, or 0.5%. To adjust for the differing amounts of pharmaceutical agent, the amount of mannitol was adjusted: 66% mannitol when 25% pharmaceutical agent was used; 86% mannitol when 5% pharmaceutical agent was used; 90.5% mannitol when 0.5% pharmaceutical agent was used. In each of these preparations, the crospovidone was used at 7%.

### Definitions

“Adjuvant” or “Adjuvant therapy” broadly refers to an agent that affects an immunological or physiological response in a subject (e.g., human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a pharmaceutical composition such as a solid dosage form of a pharmaceutical agent as described herein) to a subject. Examples of routes of administration include oral administration, rectal administration, topical administration, inhalation (nasal) or injection. Administration by injection includes intravenous (IV), intramuscular (1 M), intratumoral (IT) and subcutaneous (SC) administration. A pharmaceutical composition described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, a pharmaceutical composition described herein is administered orally, rectally, intratumorally, topically, intravesically, by injection into or adjacent to a draining lymph node, intravenously, by inhalation or aerosol, or subcutaneously. In another preferred embodiment, a pharmaceutical composition described herein is administered orally, intratumorally, or intravenously. In another embodiment, a pharmaceutical composition described herein is administered orally.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L.}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIESⓇ, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

“Cancer” broadly refers to an uncontrolled, abnormal growth of a host’s own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s) and” “neoplasm(s)” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH2ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells. Environments of particular interest are the microenvironments where cancer cells reside or locate. In some instances, the microenvironment is a tumor microenvironment or a tumor draining lymph node. In other instances, the microenvironment is a pre-cancerous tissue site or the site of local administration of a composition or a site where the composition will accumulate after remote administration.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

A “combination” of bacteria from two or more strains includes the physical co-existence of the bacteria, either in the same material or product or in physically connected products, as well as the temporal co-administration or co-localization of the bacteria from the two or more strains.

A “combination” of mEVs (such as smEVs and/or pmEVs) from two or more microbial (such as bacteria) strains includes the physical co-existence of the microbes from which the mEVs (such as smEVs and/or pmEVs) are obtained, either in the same material or product or in physically connected products, as well as the temporal co-administration or co-localization of the mEVs (such as smEVs and/or pmEVs) from the two or more strains.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size).

“Dysbiosis” refers to a state of the microbiota or microbiome of the gut or other body area, including, e.g., mucosal or skin surfaces (or any other microbiome niche) in which the normal diversity and/or function of the host gut microbiome ecological networks “microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in: a change (e.g., increase or decrease) in the prevalence of one or more bacteria types (e.g., anaerobic.), species and/or strains, change (e.g., increase of decrease) in diversity of the host microbiome population composition; a change (e.g., increase or reduction) of one or more populations of symbiont organisms resulting in a reduction or loss of one or more beneficial effects; overgrowth of one or more populations of pathogens (e.g., pathogenic bacteria); and/or the presence of and/or overgrowth of, symbiotic organisms that cause disease only when certain conditions are present.

The term “ecological consortium” is a group of bacteria which trades metabolites and positively co-regulates one another, in contrast to two bacteria which induce host synergy through activating complementary host pathways for improved efficacy.

The term “effective dose” or “effective amount” is an amount of a pharmaceutical agent that is effective to achieve a desired therapeutic response in a subject for a particular agent, composition, and mode of administration.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UW(l) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave’s disease, rheumatoid arthritis, multiple sclerosis, Goodpasture’s syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject’s immune system to treat disease (e.g., immune disease, inflammatory disease, metabolic disease, cancer) and includes, for example, checkpoint inhibitors, cancer vaccines, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10^{∧}3 fold, 10^{∧}4 fold, 10^{∧}5 fold, 10^{∧}6 fold, and/or 10^{∧}7 fold greater after treatment when compared to a pre-treatment state. Properties that may be increased include the number of immune cells, bacterial cells, stromal cells, myeloid derived suppressor cells, fibroblasts, metabolites, the level of a cytokine; or another physical parameter (such as ear thickness (e.g., in a DTH animal model) or tumor size).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′- cGAMP, 3′3′-cGAMP, c-di-AMP, c-di-GMP, 2′2′-cGAMP, and 2′3′-cGA1Vl(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR10 and TLRI1. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe (such as a bacterium), an mEV (such as an smEV and/or pmEV) or other entity or substance that has been (1) separated from at least some of the components with which it was associated when initially produced (whether in nature or in an experimental setting), and/or (2) produced, prepared, purified, and/or manufactured by the hand of man. Isolated microbes or mEVs may be separated from at least about 10%, about 20%, about 30%, about 40%, about 50%, about 60%, about 70%, about 80%, about 90%, or more of the other components with which they were initially associated. In some embodiments, isolated microbes or mEVs are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or mEV or other material that has been separated from at least some of the components with which it was associated either when initially produced or generated (e.g., whether in nature or in an experimental setting), or during any time after its initial production. A microbe or a microbial population or mEVs may be considered purified if it is isolated at or after production, such as from a material or environment containing the microbe or microbial population, and a purified microbe or microbial population or mEVs may contain other materials up to about 10%, about 20%, about 30%, about 40%, about 50%, about 60%, about 70%, about 80%, about 90%, or above about 90% and still be considered “isolated.” In some embodiments, purified microbes or microbial population or mEVs are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components (such as mEVs) thereof are generally purified from residual habitat products.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

The term “LPS mutant or lipopolysaccharide mutant” broadly refers to selected bacteria that comprises loss of LPS. Loss of LPS might be due to mutations or disruption to genes involved in lipid A biosynthesis, such as IpxA, IpxC, and IpxD. Bacteria comprising LPS mutants can be resistant to aminoglycosides and polymyxins (polymyxin B and colistin).

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbe” refers to any natural or engineered organism characterized as a archaeaon, parasite, bacterium, fungus, microscopic alga, protozoan, and the stages of development or life cycle stages (e.g., vegetative, spore (including sporulation, dormancy, and germination), latent, biofilm) associated with the organism. Examples of gut microbes include: Actinomyces graevenitzii, Actinomyces odontolyticus, Akkermansia muciniphila, Bacteroides caccae, Bacteroides fragilis, Bacteroides putredinis, Bacteroides thetaiotaomicron, Bacteroides vultagus, Bifidobacterium adolescentis, Bifidobacterium bifidum, Bilolophila wadsworthia, Blautia, Butyrivibrio, Campylobacter gracilis, Clostridia cluster III, Clostridia cluster IV, Clostridia cluster IX (Acidaminococcaceae group), Clostridia cluster XI, Clostridia cluster XIII (Peptostreptococcus group), Clostridia cluster XIV, Clostridia cluster XV, (Collinsella aerofaciens, Coprococcus, Corynebacterium sunsvallense, Desulfomonas pigra, Dorea formicigenerans, Dorea longicatena, Escherichia coli, Eubacterium hadrum, Eubacterium rectale, Faecalibacteria prausnitzii, Gemella, Lactococcus, Lanchnospira, Mollicutes cluster XVI, Mollicutes cluster XVIII, Prevotella, Rothia mucilaginosa, Ruminococcus callidus, Ruminococcus gnavus, Ruminococcus torques, and Streptococcus.

“Microbial extracellular vesicles” (mEVs) can be obtained from microbes such as bacteria, archaea, fungi, microscopic algae, protozoans, and parasites. In some embodiments, the mEVs are obtained from bacteria. mEVs include secreted microbial extracellular vesicles (smEVs) and processed microbial extracellular vesicles (pmEVs). “Secreted microbial extracellular vesicles” (smEVs) are naturally-produced vesicles derived from microbes. smEVs are comprised of microbial lipids and/or microbial proteins and/or microbial nucleic acids and/or microbial carbohydrate moieties, and are isolated from culture supernatant. The natural production of these vesicles can be artificially enhanced (e.g., increased) or decreased through manipulation of the environment in which the bacterial cells are being cultured (e.g., by media or temperature alterations). Further, smEV compositions may be modified to reduce, increase, add, or remove microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). As used herein, the term “purified smEV composition” or “smEV composition” refers to a preparation of smEVs that have been separated from at least one associated substance found in a source material (e.g., separated from at least one other microbial component) or any material associated with the smEVs in any process used to produce the preparation. It can also refer to a composition that has been significantly enriched for specific components. “Processed microbial extracellular vesicles” (pmEVs) are a non-naturally-occurring collection of microbial membrane components that have been purified from artificially lysed microbes (e.g., bacteria) (e.g., microbial membrane components that have been separated from other, intracellular microbial cell components), and which may comprise particles of a varied or a selected size range, depending on the method of purification. A pool of pmEVs is obtained by chemically disrupting (e.g., by lysozyme and/or lysostaphin) and/or physically disrupting (e.g., by mechanical force) microbial cells and separating the microbial membrane components from the intracellular components through centrifugation and/or ultracentrifugation, or other methods. The resulting pmEV mixture contains an enrichment of the microbial membranes and the components thereof (e.g., peripherally associated or integral membrane proteins, lipids, glycans, polysaccharides, carbohydrates, other polymers), such that there is an increased concentration of microbial membrane components, and a decreased concentration (e.g., dilution) of intracellular contents, relative to whole microbes. For gram-positive bacteria, pmEVs may include cell or cytoplasmic membranes. For gram-negative bacteria, a pmEV may include inner and outer membranes. pmEVs may be modified to increase purity, to adjust the size of particles in the composition, and/or modified to reduce, increase, add or remove, microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). pmEVs can be modified by adding, removing, enriching for, or diluting specific components, including intracellular components from the same or other microbes. As used herein, the term “purified pmEV composition” or “pmEV composition” refers to a preparation of pmEVs that have been separated from at least one associated substance found in a source material (e.g., separated from at least one other microbial component) or any material associated with the pmEVs in any process used to produce the preparation. It can also refer to a composition that has been significantly enriched for specific components.

“Microbiome”′ broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., precancerous or cancerous state) or treatment conditions (e.g., antibiotic treatment, exposure to different microbes). In some aspects, the microbiome occurs at a mucosal surface. In some aspects, the microbiome is a gut microbiome. In some aspects, the microbiome is a tumor microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more cancer-associated bacterial strains are present in a sample. In some embodiments, the microbiome profile indicates the relative or absolute amount of each bacterial strain detected in the sample. In some embodiments, the microbiome profile is a cancer-associated microbiome profile. A cancer-associated microbiome profile is a microbiome profile that occurs with greater frequency in a subject who has cancer than in the general population. In some embodiments, the cancer-associated microbiome profile comprises a greater number of or amount of cancer-associated bacteria than is normally present in a microbiome of an otherwise equivalent tissue or sample taken from an individual who does not have cancer.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

An “oncobiome” as used herein comprises tumorigenic and/or cancer-associated microbiota, wherein the microbiota comprises one or more of a virus, a bacterium, a fungus, a protist, a parasite, or another microbe.

“Oncotrophic” or “oncophilic” microbes and bacteria are microbes that are highly associated or present in a cancer microenvironment. They may be preferentially selected for within the environment, preferentially grow in a cancer microenvironment or hone to a said environment.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson MJ, Wang Q, O′Sullivan O, Greene-Diniz R, Cole JR, Ross RP, and O′ Toole PW. 2.010. Comparison of two next-generation sequencing technologies for resolving highly complex microbiota composition using tandem variable 16S rRNA gene regions. Nucleic Acids Res 38: e200. Konstantinidis KT, Ramette A, and Tiedje JM. 2006. The bacterial species definition in the genomic era. Philos Trans R Soc Lond B Biol Sci 361: 1929-1940. For complete genomes, MLSTs, specific genes, other than 16S, or sets of genes OTUs that share ≥ 95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis KT, Ramette A, and Tiedje JJVL 2006. The bacterial species definition in the genomic era. Philos Trans R Soc Lond B Biol Sci 361: 1929-1940. OTUs are frequently defined by comparing sequences between organisms. Generally, sequences with no more than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide,” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, the term “preventing” a disease or condition in a subject refers to administering to the subject to a pharmaceutical treatment, e.g., the administration of one or more agents (e.g., pharmaceutical agent), such that onset of at least one symptom of the disease or condition is delayed or prevented.

As used herein, a substance is “pure” if it is substantially free of other components. The terms “purify,” “purifying” and “purified” refer to an mEV (such as an smEV and/or a pmEV) preparation or other material that has been separated from at least some of the components with which it was associated either when initially produced or generated (e.g., whether in nature or in an experimental setting), or during any time after its initial production. An mEV (such as an smEV and/or a pmEV) preparation or compositions may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified mEVs (such as smEVs and/or pmEVs) are more than about 80%, about 85%, about 90%, about 91%, about 92%, about 93%, about 94%, about 95%, about 96%, about 97%, about 98%, about 99%, or more than about 99% pure. mEV (such as an smEV and/or a pmEV) compositions (or preparations) are, e.g., purified from residual habitat products.

As used herein, the term “purified mEV composition” or “mEV composition” refers to a preparation that includes mEVs (such as smEVs and/or pmEVs) that have been separated from at least one associated substance found in a source material (e.g., separated from at least one other bacterial component) or any material associated with the mEVs (such as smEVs and/or pmEVs) in any process used to produce the preparation. It also refers to a composition that has been significantly enriched or concentrated. In some embodiments, the mEVs (such as smEVs and/or pmEVs) are concentrated by 2 fold, 3-fold, 4-fold, 5-fold, 10-fold, 100-fold, 1000-fold, 10,000-fold or more than 10,000 fold.

“Residual habitat products” refers to material derived from the habitat for microbiota within or on a subject. For example, fermentation cultures of microbes can contain contaminants, e.g., other microbe strains or forms (e.g., bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold no more than its affinity for binding to a nonspecific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof refers to one in need of a treatment (or prevention) for a disease. Mammals (i.e., mammalian animals) include humans, laboratory animals (e.g., primates, rats, mice), livestock (e.g., cows, sheep, goats, pisis), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a cancer at any developmental stage, wherein any of the stages are either caused by or opportunistically supported of a cancer associated or causative pathogen, or may be at risk of developing a cancer, or transmitting to others a cancer associated or cancer causative pathogen. In some embodiments, a subject has lung cancer, bladder cancer, prostate cancer, plasmacytoma, colorectal cancer, rectal cancer, Merkel Cell carcinoma, salivary gland carcinoma, ovarian cancer, and/or melanoma. The subject may have a tumor. The subject may have a tumor that shows enhanced macropinocytosis with the underlying genomics of this process including Ras activation. In other embodiments, the subject has another cancer. In some embodiments, the subject has undergone a cancer therapy.

As used herein, a “systemic effect” in a subject treated with a pharmaceutical composition containing bacteria or mEVs (e.g., a pharmaceutical agent comprising bacteria or mEVs) of the instant invention means a physiological effect occurring at one or more sites outside the gastrointestinal tract. Systemic effect(s) can result from immune modulation (e.g., via an increase and/or a reduction of one or more immune cell types or subtypes (e.g., CD8+ T cells) and/or one or more cytokines). Such systemic effect(s) may be the result of the modulation by bacteria or mEVs of the instant invention on immune or other cells (such as epithelial cells) in the gastrointestinal tract which then, directly or indirectly, result in the alteration of activity (activation and/or deactivation) of one or more biochemical pathways outside the gastrointestinal tract The systemic effect may include treating or preventing a disease or condition in a subject.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

As used herein, a value is “greater than” another value if it is higher by any amount (e.g., each of 100, 50, 20, 12, 11, 10.6, 10.1, 10.01, and 10.001 is at least 10). Similarly, as used herein, a value is “less than” another value if it is lower by any amount (e.g., each of 1, 2, 4, 6, 8, 9, 9.2, 9.4, 9.6, 9.8, 9.9, 9.99, 9.999 is no more than 10). In contrast, as used herein, a test value “is” an anchor value when the test value rounds to the anchor value (e.g., if “an ingredient mass is 10% of a total mass,” in which case 10% is the anchor value, the test values of 9.5, 9.6, 9.7, 9.8, 9.9, 10, 10.1, 10.2, 10.3, and 10.4 would also meet the “ingredient mass is 10% of the total mass” feature).

### Bacteria

The pharmaceutical agent of the pharmaceutical compositions disclosed herein can comprise bacteria and/or microbial extracellular vesicles (mEVs) (such as smEVs and/or pmEVs). For example, the pharmaceutical agent of the pharmaceutical compositions disclosed herein can comprise a powder comprising bacteria and/or microbial extracellular vesicles (mEVs) (such as smEVs and/or pmEVs). Within a pharmaceutical agent that contains bacteria and mEVs, the mEVs can be from the same bacterial origin (e.g., same strain) as the bacteria of the pharmaceutical agent. The pharmaceutical agent can contain bacteria and/or mEVs from one or more strains.

In some embodiments, the bacteria of the pharmaceutical agent or from which the mEVs of the pharmaceutical agent are obtained are modified to reduce toxicity or other adverse effects, to enhance delivery) (e.g., oral delivery) (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the bacteria and/or mEVs (e.g., either alone or in combination with another therapeutic agent), and/or to enhance immune activation or suppression by the bacteria and/or mEVs (such as smEVs and/or pmEVs) (e.g., through modified production of polysaccharides, pili, fimbriae, adhesins). In some embodiments, the engineered bacteria described herein are modified to improve bacteria and/or mEV (such as smEV and/or pmEV) manufacturing (e.g., higher oxygen tolerance, stability, improved freeze-thaw tolerance, shorter generation times). For example, in some embodiments, the engineered bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may result in the overexpression and/or underexpression of one or more genes. The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

Examples of taxonomic groups (e.g., class, order, family, genus, species or strain) of bacteria that can be used as a source of bacteria and/or mEVs (such as smEVs and/or pmEVs) for a pharmaceutical agent described herein are provided herein (e.g., listed in Table 1, Table 2, and/or Table 3 and/or elsewhere in the specification (e.g., Table J)). In some embodiments, the bacterial strain is a bacterial strain having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein. In some embodiments, the bacteria of the pharmaceutical agent or from which the mEVs of the pharmaceutical agent are obtained are oncotrophic bacteria. In some embodiments, the bacteria of the pharmaceutical agent or from which the mEVs of the pharmaceutical agent are obtained are immunomodulatory bacteria. In some embodiments, the bacteria of the pharmaceutical agent or from which the mEVs of the pharmaceutical agent are obtained are immunostimulatory bacteria. In some embodiments, the bacteria of the pharmaceutical agent or from which the mEVs of the pharmaceutical agent are obtained are immunosuppressive bacteria. In some embodiments, the bacteria of the pharmaceutical agent or from which the mEVs of the pharmaceutical agent are obtained are immunomodulatory bacteria. In certain embodiments, the bacteria of the pharmaceutical agent or from which the mEVs of the pharmaceutical agent are obtained are generated from a combination of bacterial strains provided herein. In some embodiments, the combination is a combination of at least 2, 3, 4, 5, 6, 7, 8, 9, 10, 11, 12, 13, 14, 15, 20, 25, 30, 35, 40, 45 or 50 bacterial strains. In some embodiments, the combination includes the bacteria of the pharmaceutical agent or from which the mEVs of the pharmaceutical agent are obtained are from bacterial strains listed herein and/or bacterial strains having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein (e.g., listed in Table 1, Table 2, and/or Table 3 and/or elsewhere in the specification (e.g., Table J)). In certain embodiments, the bacteria of the pharmaceutical agent or from which the mEVs of the pharmaceutical agent are obtained are generated from a bacterial strain provided herein. In some embodiments, the bacteria of the pharmaceutical agent or from which the mEVs of the pharmaceutical agent are obtained are from a bacterial strain listed herein (e.g., listed in Table 1, Table 2, and/or Table 3 and/or elsewhere in the specification (e.g., Table J)) and/or a bacterial strain having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein (e.g., listed in Table 1, Table 2, and/or Table 3 and/or elsewhere in the specification (e.g., Table J)).

In some embodiments, the bacteria of the pharmaceutical agent or from which the mEVs of the pharmaceutical agent are obtained are Gram negative bacteria.

In some embodiments, the Gram negative bacteria belong to the class Negativicutes. The Negativicutes represent a unique class of microorganisms as they are the only diderm members of the Firmicutes phylum. These anaerobic organisms can be found in the environment and are normal commensals of the oral cavity and GI tract of humans. Because these organisms have an outer membrane, the yields of EVs from this class were investigated. It was found that on a per cell basis these bacteria produce a high number of vesicles (10-150 EVs/cell). The EVs from these organisms are broadly stimulatory and highly potent in in vitro assays. Investigations into their therapeutic applications in several oncology and inflammation in vivo models have shown their therapeutic potential. The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestine, and Propionospora sp.

In some embodiments, the bacteria of the pharmaceutical agent or from which the mEVs of the pharmaceutical agent are obtained are Gram positive bacteria.

In some embodiments, the bacteria of the pharmaceutical agent or from which the mEVs of the pharmaceutical agent are obtained are aerobic bacteria.

In some embodiments, the bacteria of the pharmaceutical agent or from which the mEVs of the pharmaceutical agent are obtained are anaerobic bacteria. In some embodiments, the anaerobic bacteria comprise obligate anaerobes. In some embodiments, the anaerobic bacteria comprise facultative anaerobes.

In some embodiments, the bacteria of the pharmaceutical agent or from which the mEVs of the pharmaceutical agent are obtained are acidophile bacteria.

In some embodiments, the bacteria of the pharmaceutical agent or from which the mEVs of the pharmaceutical agent are obtained are alkaliphile bacteria.

In some embodiments, the bacteria of the pharmaceutical agent or from which the mEVs of the pharmaceutical agent are obtained are neutral ophile bacteria.

In some embodiments, the bacteria of the pharmaceutical agent or from which the mEVs of the pharmaceutical agent are obtained are fastidious bacteria.

In some embodiments, the bacteria of the pharmaceutical agent or from which the mEVs of the pharmaceutical agent are obtained are nonfastidious bacteria.

In some embodiments, the bacteria of the pharmaceutical agent or from which the mEVs of the pharmaceutical agent are obtained or the mEVs themselves are lyophilized.

In some embodiments, the bacteria of the pharmaceutical agent or from which the mEVs of the pharmaceutical agent are obtained or the mEVs themselves are gamma irradiated (e.g., at 17.5 or 25 kGy).

In some embodiments, the bacteria of the pharmaceutical agent or from which the mEVs of the pharmaceutical agent are obtained or the mEVs themselves are UV irradiated.

In some embodiments, the bacteria of the pharmaceutical agent or from which the mEVs of the pharmaceutical agent are obtained or the mEVs themselves are heat inactivated (e.g., at 50° C. for two hours or at 90° C. for two hours).

In some embodiments, the bacteria of the pharmaceutical agent or from which the mEVs of the pharmaceutical agent are obtained or the mEVs themselves are acid treated.

In some embodiments, the bacteria of the pharmaceutical agent or from which the mEVs of the pharmaceutical agent are obtained or the mEVs themselves are oxygen sparged (e.g., at 0.1 vvm for two hours).

The phase of growth can affect the amount or properties of bacteria and/or mEVs produced by bacteria. For example, in the methods of mEVs preparation provided herein, mEVs can be isolated, e.g., from a culture, at the start of the log phase of growth, midway through the log phase, and/or once stationary phase growth has been reached.

In certain embodiments, the bacteria of the pharmaceutical agent or from which the mEVs of the pharmaceutical agent are obtained from obligate anaerobic bacteria. Examples of obligate anaerobic bacteria include gram-negative rods (including the genera of Bacteroides, Prevotella, Porphyromonas, Fusobacterium, Bilophila and Sutterella spp.), gram-positive cocci (primarily Peptostreptococcus spp.), gram-positive spore-forming (Clostridium spp.), non-spore-forming bacilli (Actinomyces, Propionibacterium, Eubacterium, Lactobacillus and Bifidobacterium spp.), and gram-negative cocci (mainly Veillonella spp.). In some embodiments, the obligate anaerobic bacteria are of a genus selected from the group consisting of Agathobaculum, Atopobium, Blautia, Burkholderia, Dielma, Longicatena, Paraclostridium, Turicibacter, and Tyzzerella.

The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestini, and Propionospora sp.

In some embodiments, the bacteria of the pharmaceutical agent or from which the mEVs of the pharmaceutical agent are obtained are of the Negativicutes class.

In some embodiments, the bacteria of the pharmaceutical agent or from which the mEVs of the pharmaceutical agent are obtained are of the Veillonellaceae family.

In some embodiments, the bacteria of the pharmaceutical agent or from which the mEVs of the pharmaceutical agent are obtained are of the Selenomonadaceae family.

In some embodiments, the bacteria of the pharmaceutical agent or from which the mEVs of the pharmaceutical agent are obtained are of the Acidaminococcaceae family.

In some embodiments, the bacteria of the pharmaceutical agent or from which the mEVs of the pharmaceutical agent are obtained are of the Sporomusaceae family.

In some embodiments, the bacteria of the pharmaceutical agent or from which the mEVs of the pharmaceutical agent are obtained are of the Megasphaera genus.

In some embodiments, the bacteria of the pharmaceutical agent or from which the mEVs of the pharmaceutical agent are obtained are of the Selenomonas genus.

In some embodiments, the bacteria of the pharmaceutical agent or from which the mEVs of the pharmaceutical agent are obtained are of the Propionospora genus.

In some embodiments, the bacteria of the pharmaceutical agent or from which the mEVs of the pharmaceutical agent are obtained are of the Acidaminococcus genus.

In some embodiments, the bacteria of the pharmaceutical agent or from which the mEVs of the pharmaceutical agent are obtained are Megasphaera sp. bacteria.

In some embodiments, the bacteria of the pharmaceutical agent or from which the mEVs of the pharmaceutical agent are obtained are Selenomonas felix bacteria.

In some embodiments, the bacteria of the pharmaceutical agent or from which the mEVs of the pharmaceutical agent are obtained are Acidaminococcus intestini bacteria.

In some embodiments, the bacteria of the pharmaceutical agent or from which the mEVs of the pharmaceutical agent are obtained are Propionospora sp. bacteria.

The Oscillospriraceae family within the Clostridia class of microorganisms are common commensal organisms of vertebrates.

In some embodiments, the bacteria of the pharmaceutical agent or from which the mEVs of the pharmaceutical agent are obtained are of the Clostridia class.

In some embodiments, the bacteria of the pharmaceutical agent or from which the mEVs of the pharmaceutical agent are obtained are of the Oscillospriraceae family.

In some embodiments, the bacteria of the pharmaceutical agent or from which the mEVs of the pharmaceutical agent are obtained are of the Faecalibacterium genus.

In some embodiments, the bacteria of the pharmaceutical agent or from which the mEVs of the pharmaceutical agent are obtained are of the Fournierella genus.

In some embodiments, the bacteria of the pharmaceutical agent or from which the mEVs of the pharmaceutical agent are obtained are of the Harryflintia genus.

In some embodiments, the bacteria of the pharmaceutical agent or from which the mEVs of the pharmaceutical agent are obtained are of the Agathobaculum genus.

In some embodiments, the bacteria of the pharmaceutical agent or from which the mEVs of the pharmaceutical agent are obtained are Faecalibacterium prausnitzii (e.g., Faecalibacterium prausnitzii Strain A) bacteria.

In some embodiments, the bacteria of the pharmaceutical agent or from which the mEVs of the pharmaceutical agent are obtained are Fournierella massiliensis (e.g., Fournierella massiliensis Strain A) bacteria.

In some embodiments, the bacteria of the pharmaceutical agent or from which the mEVs of the pharmaceutical agent are obtained are Harryflintia acetispora (e.g., Harryffintia acetispora Strain A) bacteria.

In some embodiments, the bacteria of the pharmaceutical agent or from which the mEVs of the pharmaceutical agent are obtained are Agathobaculum sp. (e.g., Agathobaculum sp. Strain A) bacteria.

In some embodiments, the bacteria of the pharmaceutical agent or from which the mEVs of the pharmaceutical agent are obtained are bacteria of a genus selected from the group consisting of Escherichia, Klebsiella, Lactobacillus, Shigella, and Staphylococcus.

In some embodiments, the bacteria of the pharmaceutical agent or from which the mEVs of the pharmaceutical agent are obtained are a species selected from the group consisting of Blautia massiliensis, Paraclostridium benzoelyticum, Diehna fastidiosa, Longicatena caecimuris, Lactococcus lactis cremoris, Tyzzerella nexilis, Hungatella effluvia, Klebsiella quasipneumoniae subsp. Similipneumoniae, Klebsiella oxytoca, and Veillonella tobetsuensis.

In some embodiments, the bacteria of the pharmaceutical agent or from which the mEVs of the pharmaceutical agent are obtained are a Prevotella bacteria selected from the group consisting of Prevotella albensis, Prevotella amnii, Prevotella, bergensis, Prevotella bivia, Prevotella brevis, Prevotella bryantii, Prevotella bitecae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivas, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, and Prevotella veroralis.

In some embodiments, the bacteria of the pharmaceutical agent or from which the mEVs of the pharmaceutical agent are obtained are a strain of bacteria comprising a genomic sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the genomic sequence of the strain of bacteria deposited with the ATCC Deposit number as provided in Table 3. In some embodiments, the bacteria of the pharmaceutical agent or from which the mEVs of the pharmaceutical agent are obtained are a strain of bacteria comprising a 16S sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the 16S sequence of the strain of bacteria deposited with the ATCC Deposit number as provided in Table 3.

The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestini, and Propionospora sp.

In some embodiments, the bacteria of the pharmaceutical agent or from which the mEVs of the pharmaceutical agent are obtained are of the Negativicutes class.

In some embodiments, the bacteria of the pharmaceutical agent or from which the mEVs of the pharmaceutical agent are obtained are of the Veillonellaceae family.

In some embodiments, the bacteria of the pharmaceutical agent or from which the mEVs of the pharmaceutical agent are obtained are of the Selenomonadaceae family.

In some embodiments, the bacteria of the pharmaceutical agent or from which the mEVs of the pharmaceutical agent are obtained are of the Acidaminococcaceae family.

In some embodiments, the bacteria of the pharmaceutical agent or from which the mEVs of the pharmaceutical agent are obtained are of the Sporornusaceae family.

In some embodiments, the bacteria of the pharmaceutical agent or from which the mEVs of the pharmaceutical agent are obtained are of the Megasphaera genus.

In some embodiments, the bacteria of the pharmaceutical agent or from which the mEVs of the pharmaceutical agent are obtained are of the Selenomonas genus.

In some embodiments, the bacteria of the pharmaceutical agent or from which the mEVs of the pharmaceutical agent are obtained are of the Propionospora genus.

In some embodiments, the bacteria of the pharmaceutical agent or from which the mEVs of the pharmaceutical agent are obtained are of the Acidaminococcus genus.

In some embodiments, the bacteria of the pharmaceutical agent or from which the mEVs of the pharmaceutical agent are obtained are Megasphaera sp. bacteria.

In some embodiments, the bacteria of the pharmaceutical agent or from which the mEVs of the pharmaceutical agent are obtained are Selenomonas felix bacteria.

In some embodiments, the bacteria of the pharmaceutical agent or from which the mEVs of the pharmaceutical agent are obtained are Acidaminococcus intestini bacteria.

In some embodiments, the bacteria of the pharmaceutical agent or from which the mEVs of the pharmaceutical agent are obtained are Propionospora sp. bacteria.

The Oscillospriraceae family within the Clostridia class of microorganisms are common commensal organisms of vertebrates.

In some embodiments, the bacteria of the pharmaceutical agent or from which the mEVs of the pharmaceutical agent are obtained are of the Clostridia class.

In some embodiments, the bacteria of the pharmaceutical agent or from which the mEVs of the pharmaceutical agent are obtained are of the Oscillospriraceae family.

In some embodiments, the bacteria of the pharmaceutical agent or from which the mEVs of the pharmaceutical agent are obtained are of the Faecalibacterium genus.

In some embodiments, the bacteria of the pharmaceutical agent or from which the mEVs of the pharmaceutical agent are obtained are of the Fournierella genus.

In some embodiments, the bacteria of the pharmaceutical agent or from which the mEVs of the pharmaceutical agent are obtained are of the Harryflintia genus.

In some embodiments, the bacteria of the pharmaceutical agent or from which the mEVs of the pharmaceutical agent are obtained are of the Agathobacitliini genus.

In some embodiments, the bacteria of the pharmaceutical agent or from which the mEVs of the pharmaceutical agent are obtained are Faecalibacterium prausnitzii (e.g., Faeccilibacterium prausnitzii Strain A) bacteria.

In some embodiments, the bacteria of the pharmaceutical agent or from which the mEVs of the pharmaceutical agent are obtained are Fournierella massiliensis (e.g., Fournierella massiliensis Strain A) bacteria.

In some embodiments, the bacteria of the pharmaceutical agent or from which the mEVs of the pharmaceutical agent are obtained are Harryflintia acetispora (e.g., Harryflintia acetispora Strain A) bacteria.

In some embodiments, the bacteria of the pharmaceutical agent or from which the mEVs of the pharmaceutical agent are obtained are Agathobaculum sp. (e.g., Agathobaculum sp. Strain A) bacteria.

In some embodiments, the bacteria of the pharmaceutical agent or from which the mEVs of the pharmaceutical agent are obtained are a strain of Agathobaculum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA- 125892).

In some embodiments, the bacteria of the pharmaceutical agent or from which the mEVs of the pharmaceutical agent are obtained are of the class Bacteroidia [phylum Bacteroidota]. In some embodiments, the bacteria of the pharmaceutical agent or from which the mEVs of the pharmaceutical agent are obtained are bacteria of order Bacteroidales. In some embodiments, the bacteria of the pharmaceutical agent or from which the mEVs of the pharmaceutical agent are obtained are of the family Porphyromonoadaceae. In some embodiments, the bacteria of the pharmaceutical agent or from which the mEVs of the pharmaceutical agent are obtained are of the family Prevotellaceae. In some embodiments, the bacteria of the pharmaceutical agent or from which the mEVs of the pharmaceutical agent are obtained are bacteria of the class Bacteroidia wherein the cell envelope structure of the bacteria is diderm. In some embodiments, the bacteria of the pharmaceutical agent or from which the mEVs of the pharmaceutical agent are obtained are bacteria of the class Bacteroidia that stain Gram negative. In some embodiments, the bacteria of the pharmaceutical agent or from which the mEVs of the pharmaceutical agent are obtained are bacteria of the class Bacteroidia wherein the bacteria is diderm and the bacteria stain Gram negative.

In some embodiments, the bacteria of the pharmaceutical agent or from which the mEVs of the pharmaceutical agent are obtained are bacteria of the class Clostridia [phylum Firmicutes]. In some embodiments, the bacteria of the pharmaceutical agent or from which the mEVs of the pharmaceutical agent are obtained are of the order Eubacteriales. In some embodiments, the bacteria of the pharmaceutical agent or from which the mEVs of the pharmaceutical agent are obtained are of the family Oscillispiraceae. In some embodiments, the bacteria of the pharmaceutical agent or from which the mEVs of the pharmaceutical agent are obtained are of the family Lachnospiraceae. In some embodiments, the bacteria of the pharmaceutical agent or from which the mEVs of the pharmaceutical agent are obtained are of the family Peptostreptococcaceae. In some embodiments, the bacteria of the pharmaceutical agent or from which the mEVs of the pharmaceutical agent are obtained are of the family Clostridialesfamily XIII/Incertae sedis41. In some embodiments, the bacteria of the pharmaceutical agent or from which the mEVs of the pharmaceutical agent are obtained are of the class Clostridia wherein the cell envelope structure of the bacteria is monoderm. In some embodiments, the bacteria of the pharmaceutical agent or from which the mEVs of the pharmaceutical agent are obtained are of the class Clostridia that stain Gram negative. In some embodiments, the bacteria of the pharmaceutical agent or from which the mEVs of the pharmaceutical agent are obtained are of the class Clostridia that stain Gram positive. In some embodiments, the bacteria of the pharmaceutical agent or from which the mEVs of the pharmaceutical agent are obtained are of the class Clostridia wherein the cell envelope structure of the bacteria is monoderm and the bacteria stain Gram negative. In some embodiments, the bacteria of the pharmaceutical agent or from which the mEVs of the pharmaceutical agent are obtained are of the class Clostridia wherein the cell envelope structure of the bacteria is monoderm and the bacteria stain Gram positive.

In some embodiments, the bacteria of the pharmaceutical agent or from which the mEVs of the pharmaceutical agent are obtained are of the class Negativicutes [phylum Firmicutes]. In some embodiments, the bacteria of the pharmaceutical agent or from which the mEVs of the pharmaceutical agent are obtained are of the order Veillonellales. In some embodiments, the bacteria of the pharmaceutical agent or from which the mEVs of the pharmaceutical agent are obtained are of the family Veillonelloceae. In some embodiments, the bacteria of the pharmaceutical agent or from which the mEVs of the pharmaceutical agent are obtained are of the order Selenomonadales. In some embodiments, the bacteria of the pharmaceutical agent or from which the mEVs of the pharmaceutical agent are obtained are bacteria of the family Selenomonadaceae. In some embodiments, the bacteria of the pharmaceutical agent or from which the mEVs of the pharmaceutical agent are obtained are of the family Sporomusaceae. In some embodiments, t the bacteria of the pharmaceutical agent or from which the mEVs of the pharmaceutical agent are obtained are of the class Negativicutes wherein the cell envelope structure of the bacteria is diderm. In some embodiments, the bacteria of the pharmaceutical agent or from which the mEVs of the pharmaceutical agent are obtained are of the the bacteria of the pharmaceutical agent or from which the mEVs of the pharmaceutical agent are obtained are the EVs are from bacteria of the class Negativicutes wherein the cell envelope structure of the bacteria is diderm and the bacteria stain Gram negative.

In some embodiments, the bacteria of the pharmaceutical agent or from which the mEVs of the pharmaceutical agent are obtained are of the class Synergistia [phylum Synergistota]. In some embodiments, the bacteria of the pharmaceutical agent or from which the mEVs of the pharmaceutical agent are obtained are of the order Synergistales. In some embodiments, the bacteria of the pharmaceutical agent or from which the mEVs of the pharmaceutical agent are obtained are of the family Synergistaceae. In some embodiments, the bacteria of the pharmaceutical agent or from which the mEVs of the pharmaceutical agent are obtained are of the class Synergistia wherein the cell envelope structure of the bacteria is diderm. In some embodiments, the bacteria of the pharmaceutical agent or from which the mEVs of the pharmaceutical agent are obtained are of the class Synergistia that stain Gram negative. In some embodiments, the bacteria of the pharmaceutical agent or from which the mEVs of the pharmaceutical agent are obtained are of the class Synergistia wherein the cell envelope structure of the bacteria is diderm and the bacteria stain Gram negative.

In some embodiments, the bacteria of the pharmaceutical agent or from which the mEVs of the pharmaceutical agent are obtained are from one strain of bacteria, e.g., a strain provided herein.

In some embodiments, the bacteria of the pharmaceutical agent or from which the mEVs of the pharmaceutical agent are obtained are from one strain of bacteria (e.g., a strain provided herein) or from more than one strain provided herein.

In some embodiments, the bacteria of the pharmaceutical agent or from which the mEVs of the pharmaceutical agent are obtained are Lactococcus lactis cremoris bacteria, e.g., a strain comprising at least 90% or at least 99% genomic, 16S and/or CRISPR sequence identity to the nucleotide sequence of the Lactococcus lactis cremoris Strain A (ATCC designation number PTA-125368). In some embodiments, the bacteria of the pharmaceutical agent or from which the mEVs of the pharmaceutical agent are obtained are Lactococcus bacteria, e.g., Lactococcus lactis cremoris Strain A (ATCC designation number PTA-125368).

In some embodiments, the bacteria of the pharmaceutical agent or from which the mEVs of the pharmaceutical agent are obtained are Prevotella bacteria, e.g., a strain comprising at least 90% or at least 99% genomic, 16S and/or CRISPR sequence identity to the nucleotide sequence of the Prevotella Strain B 50329 (NRRL accession number B 50329). In some embodiments, the bacteria of the pharmaceutical agent or from which the mEVs of the pharmaceutical agent are obtained are Prevotella bacteria, e.g., Prevotella Strain B 50329 (NRRL accession number B 50329).

In some embodiments, the bacteria of the pharmaceutical agent or from which the mEVs of the pharmaceutical agent are obtained are Bifidobacterium bacteria, e.g., a strain comprising at least 90% or at least 99% genomic, 16S and/or CRISPR sequence identity to the nucleotide sequence of the Bifidobacterium bacteria deposited as ATCC designation number PTA-125097. In some embodiments, the bacteria of the pharmaceutical agent or from which the mEVs of the pharmaceutical agent are obtained are Bifidobacterium bacteria, e.g., Bifidobacterium bacteria deposited as ATCC designation number PTA-125097.

In some embodiments, the bacteria of the pharmaceutical agent or from which the mEVs of the pharmaceutical agent are obtained are Veillonella bacteria, e.g., a strain comprising at least 90% or at least 99% genomic, 16S and/or CRISPR sequence identity to the nucleotide sequence of the Veillonella bacteria deposited as ATCC designation number PTA-125691. In some embodiments, the bacteria of the pharmaceutical agent or from which the mEVs of the pharmaceutical agent are obtained are Veillonella bacteria, e.g., Veillonella bacteria deposited as ATCC designation number PTA-125691.

In some embodiments, the bacteria of the pharmaceutical agent or from which the mEVs of the pharmaceutical agent are obtained are Ruminococcus gnavus bacteria. In some embodiments, the Ruminococcus gnavus bacteria are a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are Ruminococcus gnavus bacteria deposited as ATCC designation number PTA-126695.

In some embodiments, the bacteria of the pharmaceutical agent or from which the mEVs of the pharmaceutical agent are obtained are Megasphaera sp. bacteria. In some embodiments, the Megasphaera sp. bacteria are a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are Megasphaera sp. bacteria deposited as ATCC designation number PTA-126770.

In some embodiments, the bacteria of the pharmaceutical agent or from which the mEVs of the pharmaceutical agent are obtained are Fournierella massiliensis bacteria. In some embodiments, the Fournierella massiliensis bacteria are a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are Fournierella massiliensis bacteria deposited as ATCC designation number PTA-126696.

In some embodiments, the bacteria of the pharmaceutical agent or from which the mEVs of the pharmaceutical agent are obtained are Hanyflintia acetispora bacteria. In some embodiments, the Harryflintia acetispora bacteria are a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are Harryflintia acetispora bacteria deposited as ATCC designation number PTA-126694.

In some embodiments, the bacteria of the pharmaceutical agent or from which the mEVs of the pharmaceutical agent are obtained are bacteria that produce metabolites, e.g., the bacteria produce butyrate, iosine, proprionate, or tryptophan metabolites.

In some embodiments, the bacteria of the pharmaceutical agent or from which the mEVs of the pharmaceutical agent are obtained are bacteria that produce butyrate. In some embodiments, the bacteria of the pharmaceutical agent or from which the mEVs of the pharmaceutical agent are obtained are from the genus Blautia; Christensella; Copracoccus; Eubacterium; Lachnosperacea; Megasphaera; or Roseburia.

In some embodiments, the bacteria of the pharmaceutical agent or from which the mEVs of the pharmaceutical agent are obtained are bacteria that produce iosine. In some embodiments, the bacteria of the pharmaceutical agent or from which the mEVs of the pharmaceutical agent are obtained are from the genus Bifidobacterium; Lactobacillus; or Olsenella.

In some embodiments, the bacteria of the pharmaceutical agent or from which the mEVs of the pharmaceutical agent are obtained are bacteria that produce proprionate. In some embodiments, the bacteria of the pharmaceutical agent or from which the mEVs of the pharmaceutical agent are obtained are from the genus Akkermansia; Bacteriodes; Dialister; Eubacterium; Megasphaera; Parabacteriodes; Prevotella; Ruminococcus; or Veillonella.

In some embodiments, the bacteria of the pharmaceutical agent or from which the mEVs of the pharmaceutical agent are obtained are bacteria that produce tryptophan metabolites. In some embodiments, the bacteria of the pharmaceutical agent or from which the mEVs of the pharmaceutical agent are obtained are from the genus Lactobacillus or Peptostreptococcus.

In some embodiments, the bacteria of the pharmaceutical agent or from which the mEVs of the pharmaceutical agent are obtained are bacteria that produce inhibitors of histone deacetylase 3 (HDAC3). In some embodiments, the bacteria of the pharmaceutical agent or from which the mEVs of the pharmaceutical agent are obtained are from the species Bariatricus massiliensis, Faecalibacterium prausnitzii, Megasphaera massiliensis or Roseburia intestinalis.

TABLE 1

Bacteria by Class

| | | | | |
|---|---|---|---|---|
| Class | Order | Family | Genus* | Species |
| Actinobacter | Actinomycetales | Mycobacteriaceae | Mycobacterium | |
| | | Streptomycetaceae | Streptomyces (S.) | S. lividans, S coelicolor, S sudanesis, S somaliensis |
| | Bifidobacteriales | Bifidobacteriaceae | Bifidobacterium (B.) | B. adolescentis, B.animalis, B.bifidum, B. breve, B. lactis, B. longum, B. pseudocatenulatum |
| | Coriobacteriales | Coriobacteriaceae | Collinsella | Collinsella aerofaciens |
| | | | Olsenella | Olsenella faecalis |
| | Propionibacteriales | Propionibacteraceae | Propionibacterium | |
| Bacilli | Bacillales | Bacillalesincertae sedis family XI | Gemella (G.) | G. haemolysans, G. morbillorum |
| | | Listeraceae | Listeria (L.) | L. monocytogenes, L. welshimeri |
| | Lactobacillales | Enterococcaceae | Enterococcus (E.) | E. durans, E. faecium, E. faecalis, E. gallinarum, E. villorum, |
| | | | Lactobacillus (L.) | L. casei, L. fermentum, L. mucosae, L. plantarum, L. reuteri, L. rhamnosus, L. salvarius |
| | | Streptococcaceae | Lactococcus | Lactococcus lactis cremoris |
| | | | Staphylococcus | Staphylococcusaureus |
| | | | Streptococcus (S.) | S. agalactiae, S. aureus, S. australi, S. mutans, S. parasanguinis, S. pneumoniae, S. pyogenes, S. salivarius |
| Bacteriodes | Bacteroidales | Bacteriodaceae | Bacteriodes (B.) | B. caccae, B. cellulosilyticus, B. coprocola, B. dorei, B.fragilis, B. ovatus, B. putredinis, B. salanitronis, B. thetaiotaomicron, B. vulgatus |
| | | Odoribacteraceae | Odoribacter | Odoribacter splanchnicus |
| | | Porphyromonad aceae | Parabacteriodes (P.) | P. distasonis, P. goldsteinii, P. merdae |
| | | | Porphyromonas | Porphyromonas gingivalis |
| | | Prevotellaceae | Prevotella (P.) | P. albensis, P. amnii, P. aurantiaca, P. baroniae, P. bergensis, P. bivia, P. brevis, P. bryantii, P. buccae, P. buccalis, P. colorans, P. corporis, P. copri, P. dentalis, P. dentasini, P. denticola, P. disiens,, P. enoeca, P. falsenii, P. fusca, P. heparinolytica, P. histicola, P. intermedia, P. jejuni, , P. loescheii, P. |
| | | | | maculosa, P. marshii, P. melaninogenica, P. micans, P. multiformis, P. multisaccharivorax, P. nanceiensis, P. nigrescens, P. oralis, P. oris, , P. oryzae, P. oulorum, P. pallens, P. paludivivens, P. pleuritidisP. ruminicola, P. saccharolytica, P. salivae, P. scopos, P. shahii, P. stercorea, P. tannerae, P. timonensis, P. veroralis, P. zoogleoformans |
| | | Rikenellaceae | Alstipes (A.) | A. communis, A. dispar, A. finegoldii, A. indisunctus. A. ihumii, A. inops, A. massiliensis, A. megaguti, A. obesi , A. onderdonkii, A. provencensis, A. putredinis, A. senegalensis, A. shahii, A. timonensis |
| Betaproteobacteria | Burkholderiales | Alcaligenaceae | Paenalcaligenes | Paenalcaligenes hominis |
| | | | Bordella | Bordella pertussis |
| | | Burkholderiaceae | Burkholderia (B.) | B. mallei, B. pseudomallei |
| | | | Ralstonia | Ralstonia solanacea rum |
| | | Neisseriaceae | Neisseria | Neisseria meningitidis |
| | | Sutterellaceae | Sutterella (S.) | S. parvirubra, S. stercoricanis, S. wadsworthensis |
| Clostridia | Clostridiales | Catabacteriaceae | Catabacter | Catabacter hongkongensis |
| | | Clostridiaceae | Aminiphila | Anaerosphaera aminiphila |
| | | | Christensenell aceae (C.) | C. massiliensis, C. minuta, C. timonensis |
| | | | Hungatella | Hungatella effluvia |
| | | Eubacteriaceae | Eubacterium (E.) | E. contortum, E. eligens, E. faecium, E. hadrum, E. hallii, E. limosum, E. ramulus, E. rectale |
| | | Lachnospiraceae | Anaerostipes (A.) | A. caccae, A. hadrus |
| | | | Blautia (B.) | B. hydrogenotrophica, B. massiliensis, B. stercoris, B. wexlerae |
| | | | Catonella | Catonella morbi |
| | | | Coprococcus (C.) | C. catus, C. comes, C. eutactus |
| | | | Dialister (D.) | D. invisus, D. micraeophilus, D. succinatiphilus |
| | | | Dorea (D.) | D. formicigenerans, D. longicatena |
| | | | Johnsonella | Johnsonella ignava |
| | | | Oribacterium (O.) | O. parvum, O. sinus |
| | | | Lachnobacterium | |
| | | | Lachnoclostridium | |
| | | | Lacrimispora (L.) | L. sacchaarolytica |
| | | | Roseburia (R.) | R. hominis, R. intestinalis |
| | | | Tyzzerella | Tyzzerella nexilis |
| | | Oscillospiraceae | Oscillibacter | Oscillibacter valericigenes |
| | | | Harryflintia | Harryflinta acetispora |
| | | Peptococcaceae | | |
| | | Peptostreptococcaceae | Paraclostridium | Paraclostridium benzoelyticum |
| | | | Peptostreptococcus | Peptostreptococcus russellii |
| | | Ruminococcaceae | Agathobaculum | Agathobaculum sp. |
| | | | Fournierella | Fournierella masssiliensis |
| | | | Ruminococcus (R.) | R. albus, R. bromii, R. callidus, R. gnavus, R. inulinivorans, R. obeum, R. torques |
| | | | Faecalibacterium | Faecalibacterium prasusnitzii |
| | | Clostridiales family XIII/ Incertae sedis | | Intestimonas butyriciproducens |
| Fusobacteria | Fusobacteriales | Fusobacteriaceae | Fusobacterium (F) | F. nucleatum, F. naviforme |
| | | Leptotrichiaceae | Leptotrichia | |
| | | | Sneathia | |
| Gammaproteobacteria | Enterobacterales | Enterobacteriaceae | Klebsiella (K.) | K. oxytoca, K. pneumoniae, K. quasipneumoniae subsp.Similipneumoniae, |
| | | | Escherichia (E.) | E. coli strain Nissle 1917 (EcN), E. coli strain ECOR12, E. coli strain ECOR63 |
| | | | Shigella | |
| Negativicutes | | Acidaminococcaceae | Acidaminococcus (A.) | A. fermentans, A. intestine |
| | | | Phascolarctobacterium (P.) | P. faecium, P. succinatutens |
| | | Selenomonadaceae | Selenomonas (S.) | S. felix, S. incertae sedis, S. sputigena |
| | | Sporomusaceae | Selenomonadales | |
| | | Veillonellaceae | Allisonella | |
| | | | Anaeroglobus | Anaeroglobus germinatus |
| | | | Caecibacter | |
| | | | Colibacter | |
| | | | Megasphaera (M.) | M. elsedenii, M. massiliensis, M. micronuciformis, Megasphaera sp |
| | | | Massilibacillus | Massilibacillus massiliensis |
| | | | Propionispira | |
| | | | Negativicoccus | Negativicoccus succinicivornas |
| | | | Veillonella (V.) | V. dispar, V. parvula, V. ratti,V. tobetsuensis |
| | Synergistales | Synergistaceae | Aminobacterium | Aminobacterium mobile |
| | | | Cloacibacillus | Cloacibacillus evryensis |
| | | | Rarimicrobium | Rarimicrobium hominis |
| Verrucomicrobia | Verrucomicrobiales | Akkermansiaceae | Akkermansia | Akkermansia mucinophila |

^{∗} The abbreviation given in the parenthetical is for the species in the row in which it is listed.

TABLE 2

Exemplary Bacterial Strains

| | | | |
|---|---|---|---|
| OTU | Public DB Accession | OTU | Public DB Accession |
| Actinobacillus actinomycetemcomitans | AY362885 | Lactobacillus murinus | NR_042231 |
| Actinobacillus minor | ACFT01000025 | Lactobacillus nodensis | NR_041629 |
| Actinobacillus pleuropneumoniae | NR_074857 | Lactobacillus oeni | NR_043095 |
| Actinobacillus succinogenes | CP000746 | Lactobacillus oris | AEKL01000077 |
| Actinobacillus ureae | AEVG01000167 | Lactobacillus parabrevis | NR 042456 |
| Actinobaculum massiliae | AF487679 | Lactobacillus parabuchneri | NR_041294 |
| Actinobaculum schaalii | AY957507 | Lactobacillus paracasei | ABQV01000067 |
| Actinobaculum sp. BM#101342 | AY282578 | Lactobacillus parakefiri | NR_029039 |
| Actinobaculum sp. P2P_19 P1 | AY207066 | Lactobacillus pentosus | JN813103 |
| Akkermansia muciniphila | CP001071 | Lactobacillus perolens | NR_029360 |
| Alistipes finegoldii | NR_043064 | Lactobacillus plantarum | ACGZ02000033 |
| Alistipes indistinctus | AB490804 | Lactobacillus pontis | HM218420 |
| Alistipes onderdonkii | NR_043318 | Lactobacillus reuteri | ACGW02000012 |
| Alistipes putredinis | ABFK02000017 | Lactobacillus rhamnosus | ABWJ01000068 |
| Alistipes shahii | FP929032 | Lactobacillus rogosae | GU269544 |
| Alistipes sp. HGB5 | AENZ01000082 | Lactobacillus ruminis | ACGS02000043 |
| Alistipes sp. JC50 | JF824804 | Lactobacillus sakei | DQ989236 |
| Alistipes sp. RMA 9912 | GQ140629 | Lactobacillus salivarius | AEBA01000145 |
| Anaerostipes caccae | ABAX03000023 | Lactobacillus saniviri | AB602569 |
| Anaerostipes sp. 3 2 56FAA | ACWB01000002 | Lactobacillus senioris | AB602570 |
| Bacillus aeolius | NR_025557 | Lactobacillus sp. 66c | FR681900 |
| Bacillus aerophilus | NR_042339 | Lactobacillus sp. BT6 | HQ616370 |
| Bacillus aestuarii | GQ980243 | Lactobacillus sp. KLDS 1.0701 | EU600905 |
| Bacillus alcalophilus | X76436 | Lactobacillus sp. KLDS 1.0702 | EU600906 |
| Bacillus amyloliquefaciens | NR_075005 | Lactobacillus sp. KLDS 1.0703 | EU600907 |
| Bacillus anthracis | AAEN01000020 | Lactobacillus sp. KLDS 1.0704 | EU600908 |
| Bacillus atrophaeus | NR_075016 | Lactobacillus sp. KLDS 1.0705 | EU600909 |
| Bacillus badius | NR_036893 | Lactobacillus sp. KLDS 1.0707 | EU600911 |
| Bacillus cereus | ABDJ01000015 | Lactobacillus sp. KLDS 1.0709 | EU600913 |
| Bacillus circulans | AB271747 | Lactobacillus sp. KLDS 1.0711 | EU600915 |
| Bacillus clausii | FN397477 | Lactobacillus sp. KLDS 1.0712 | EU600916 |
| Bacillus coagulans | DQ297928 | Lactobacillus sp. KLDS 1.0713 | EU600917 |
| Bacillus firmus | NR_025842 | Lactobacillus sp. KLDS 1.0716 | EU600921 |
| Bacillus flexus | NR_024691 | Lactobacillus sp. KLDS 1.0718 | EU600922 |
| Bacillus fordii | NR_025786 | Lactobacillus sp. KLDS 1.0719 | EU600923 |
| Bacillus gelatini | NR_025595 | Lactobacillus sp. oral clone HT002 | AY349382 |
| Bacillus halmapalus | NR_026144 | Lactobacillus sp. oral clone HT070 | AY349383 |
| Bacillus halodurans | AY144582 | Lactobacillus sp. oral taxon 052 | GQ422710 |
| Bacillus herbersteinensis | NR_042286 | Lactobacillus tucceti | NR_042194 |
| Bacillus horti | NR_036860 | Lactobacillus ultunensis | ACGU01000081 |
| Bacillus idriensis | NR_043268 | Lactobacillus vaginalis | ACGV01000168 |
| Bacillus lentus | NR_040792 | Lactobacillus vini | NR_042196 |
| Bacillus licheniformis | NC_006270 | Lactobacillus vitulinus | NR_041305 |
| Bacillus megaterium | GU252124 | Lactobacillus zeae | NR_037122 |
| Bacillus nealsonii | NR_044546 | Lactococcus garvieae | AF061005 |
| Bacillus niabensis | NR_043334 | Lactococcus lactis | CP002365 |
| Bacillus niacini | NR_024695 | Lactococcus raffinolactis | NR_044359 |
| Bacillus pocheonensis | NR_041377 | Listeria grayi | ACCR02000003 |
| Bacillus pumilus | NR_074977 | Listeria innocua | JF967625 |
| Bacillus safensis | JQ624766 | Listeria ivanovii | X56151 |
| Bacillus simplex | NR_042136 | Listeria monocytogenes | CP002003 |
| Bacillus sonorensis | NR_025130 | Listeria welshimeri | AM1263198 |
| Bacillus sp. 10403023 MM10403188 | CAET01000089 | Megasphaera elsdenii | AY038996 |
| Bacillus sp. 2_A_57_CT2 | ACWD01000095 | Megasphaera genomosp. C1 | AY278622 |
| Bacillus sp. 2008724126 | GU252108 | Megasphaera genomosp. type 1 | ADGP01000010 |
| Bacillus sp. 2008724139 | GU252111 | Megasphaera micronuciformis | AECS01000020 |
| Bacillus sp. 7_16AIA | FN397518 | Megasphaera sp. BLPYG 07 | HM990964 |
| Bacillus sp. 9_3AIA | FN397519 | Megasphaera sp. UPII 199 6 | AFIJ01000040 |
| Bacillus sp. AP8 | JX101689 | Microbacterium gubbeenense | NR_025098 |
| Bacillus sp. B27(2008) | EU362173 | Microbacterium lacticum | EU714351 |
| Bacillus sp. BT1B CT2 | ACWC01000034 | Mitsuokella jalaludinii | NR_028840 |
| Bacillus sp. GB1.1 | FJ897765 | Mitsuokella multacida | ABWK02000005 |
| Bacillus sp. GB9 | FJ897766 | Mitsuokella sp. oral taxon 521 | GU413658 |
| Bacillus sp. HU19.1 | FJ897769 | Mitsuokella sp. oral taxon G68 | GU432166 |
| Bacillus sp. HU29 | FJ897771 | Mycobacterium abscessus | AGQU01000002 |
| Bacillus sp. HU33.1 | FJ897772 | Mycobacterium africanum | AF480605 |
| Bacillus sp. JC6 | JF824800 | Mycobacterium alsiensis | AJ938169 |
| Bacillus sp. oral taxon F26 | HM099642 | Mycobacterium avium | CP000479 |
| Bacillus sp. oral taxon F28 | HM099650 | Mycobacterium chelonae | AB548610 |
| Bacillus sp. oral taxon F79 | HM099654 | Mycobacterium colombiense | AM062764 |
| Bacillus sp. SRC_DSF1 | GU797283 | Mycobacterium elephantis | AF385898 |
| Bacillus sp. SRC_DSF10 | GU797292 | Mycobacterium gordonae | GU142930 |
| Bacillus sp. SRC_DSF2 | GU797284 | Mycobacterium intracellulare | GQ1532.76 |
| Bacillus sp. SRC DSF6 | GU797288 | Mycobacterium kansasii | AF480601 |
| Bacillus sp. tc09 | HQ844242 | Mycobacterium lacus | NR_025175 |
| Bacillus sp. zh168 | FJ851424 | Mycobacterium leprae | FM211192 |
| Bacillus sphaericus | DQ286318 | Mycobacterium lepromatosis | EU203590 |
| Bacillus sporothermodurans | NR_026010 | Mycobacterium mageritense | FR798914 |
| Bacillus subtilis | EU627588 | Mycobacterium mantenii | FJ042897, |
| Bacillus thermoamylovorans | NR_029151 | Mycobacterium marinum | NC_010612 |
| Bacillus weihenstephanensis | NR_074926 | Mycobacterium microti. | NR_025234 |
| Bacteroidales bacterium ph8 | JN837494 | Mycobacterium neoaurum | AF268445 |
| Bacteroidales genomosp. P1 | AY341819 | Mycobacterium parascrofulaceum | ADNV01000350 |
| Bacteroidales genomosp. P2 oral clone MB1 G13 | DQ003613 | Mycobacterium paraterrae | EU919229 |
| Bacteroidales genomosp. P3 oral clone MB1 G34 | DQ003615 | Mycobacterium phlei | GUI42920 |
| Bacteroidales genomosp. P4 oral cloneMB2 G17 | DQ003617 | Mycobacterium seoulense | DQ536403 |
| Bacteroidales genomosp. P5 oral clone MB2 P04 | DQ003619 | Mycobacterium smegmatis | CP000480 |
| Bacteroidales genomosp. P6 oral cloneMB3 C19 | DQ003634 | Mycobacterium sp. 1761 | U703150 |
| Bacteroidales genomosp. P7 oral clone MBP19 | DQ003623 | Mycobacterium sp. 1776 | EU703152 |
| Bacteroidales genomosp. P8 oral clone MB4 G15 | DQ003626 | Mycobacterium sp. 1781 | EU703147 |
| Bacteroides acidifaciens | NR_028607 | Mycobacterium sp. 1791 | EU703148 |
| Bacteroides barnesiae | NR_041446 | Mycobacterium sp. 1797 | EU703149 |
| Bacteroides caccae | EU136686 | Mycobacterium sp. AQ1GA4 | HM210417 |
| Bacteroides cellulosilyticus | ACCH01000108 | Mycobacterium sp. B1007.09.0206 | HQ174245 |
| Bacteroides clarus | AFBMO1000011 | Mycobacterium sp. GN 10546 | FJ497243 |
| Bacteroides coagulans | AB547639 | Mycobacterium sp. GN 10827 | FJ497247 |
| Bacteroides coprocola | ABIY02000050 | Mycobacterium sp. GN 11124 | FJ652846 |
| Bacteroides coprophilus | ACBW01000012 | Mycobacterium sp. GN 9188 | FJ497240 |
| Bacteroides dorei | ABWZ01000093 | Mycobacterium sp. GR 2007 210 | FJ555538 |
| Bacteroides eggerthii | ACWGO1000065 | Mycobacterium sp. HE5 | AJ012738 |
| Bacteroides faecis | GQ496624 | Mycobacterium sp. NLA001000736 | HM627011 |
| Bacteroides finegoldii | AB222699 | Mycobacterium sp. W | DQ437715 |
| Bacteroides fluxus | AFBN01000029 | Mycobacterium tuberculosis | CP001658 |
| Bacteroidesfragilis | A13006841 | Mycobacterium ulcerans | AB548725 |
| Bacteroides galacturonicus | DQ497994 | Mycobacterium vulneris | EU834055 |
| Bacteroides helcogenes | CP002352 | Mycoplasma, agalactiae | AF010477 |
| Bacteroides heparinolyticus | JN867284 | Mycoplasma amphoriforme | AY531656 |
| Bacteroides intestinalis | ABJL02000006 | Mycoplasma arthritidis | NC_011025 |
| Bacteroides massiliensis | AB200226 | Mycoplasma bovoculi | NR_025987 |
| Bacteroides nordii | NR_043017 | Mycoplasma faucium | NR_024983 |
| Bacteroides oleiciplenus | AB547644 | Mycoplasma fermentans | CP002458 |
| Bacteroides ovatus | ACWH01000036 | Mycoplasma flocculare | X62699 |
| Bacteroides pectinophilus | ABVQ01000036 | Mycoplasma genitalium | L43967 |
| Bacteroides plebeius | AB200218 | Mycoplasma hominis | AF443616 |
| Bacteroides pyogenes | NR_041280 | Mycoplasma orale | AY796060 |
| Bacteroides salanitronis | CP002530 | Mycoplasma ovipneumoniae | NR_025989 |
| Bacteroides salyersiae | EU136690 | Mycoplasma penetrans | NC_004432 |
| Bacteroides sp. 1_1_14 | ACRPO1000155 | Mycoplasma pneumoniae | NC_000912 |
| Bacteroides sp. 1_1_30 | ADCLO1000128 | Mycoplasma putrefaciens | U26055 |
| Bacteroides sp. 1 1 6 | ACIC01000215 | Mycoplasma salivarium | M24661 |
| Bacteroides sp. 2_1_22 | ACPQ01000117 | Mycoplasmataceae genomosp. P1 oral clone MB1 G23 | DQ003614 |
| Bacteroides sp. 2 1 56FAA | ACW101000065 | Neisseria bacilliformis | AFAY01000058 |
| Bacteroides sp. 2 2 4 | ABZZ01000168 | Neisseria cinerea | ACDY01000037 |
| Bacteroides sp. 20 3 | ACRQ01000064 | Neisseria elongata | ADBF01000003 |
| Bacteroides sp. 3 1 19 | ADCJ01000062 | Neisseria flavescens | ACQV01000025 |
| Bacteroides sp. 3_1_23 | ACRS01000081 | Neisseria genomosp. P2 oral clone MB5 P15 | DQ003630 |
| Bacteroides sp. 3 1 33FAA | ACPS01000085 | Neisseria gonorrhoeae | CP002440 |
| Bacteroides sp. 3 1 40A | ACRT01000136 | Neisseria lactamica | ACEQ01000095 |
| Bacteroides sp. 3 2 5 | ACIB01000079 | Neisseria macacae | AFQE01000146 |
| Bacteroides sp. 315 5 | FJ848547 | Neisseria meningitidis | NC_003112 |
| Bacteroides sp. 31SF15 | AJ583248 | Neisseria mucosa | ACDX01000110 |
| Bacteroides sp. 31SF18 | AJ583249 | Neisseria pharyngis | AJ239281 |
| Bacteroides sp. 35AE31 | AJ583244 | Neisseria polysaccharea | ADBE01000137 |
| Bacteroides sp. 35AE37 | AJ583245 | Neisseria sicca | ACK002000016 |
| Bacteroides sp. 35BE34 | AJ583246 | Neisseria sp. KEM232 | GQ203291 |
| Bacteroides sp. 35BE35 | AJ583247 | Neisseria sp. oral clone AP132 | AY005027 |
| Bacteroides sp. 4_1_36 | ACTC01000133 | Neisseria sp. oral clone JC012 | AY349388 |
| Bacteroides sp. 4_3_47FAA | ACDR02000029 | Neisseria sp. oral strain B33KA | AY005028 |
| Bacteroides sp. 9_1_42FAA | ACAA01000096 | Neisseria sp. oral taxon 014 | ADEA01000039 |
| Bacteroides sp. AR20 | AF139524 | Neisseria sp. SMC A9199 | FJ763637 |
| Bacteroides sp. AR29 | AF139525 | Neisseria sp. TM10 1 | DQ279352 |
| Bacteroides sp. B2 | EU722733 | Neisseria subflava | ACEO01000067 |
| Bacteroides sp. D1 | ACAB02000030 | Odoribacter laneus | AB490805 |
| Bacteroides sp. D2 | ACGA01000077 | Odoribacter splanchnicus | CP002544 |
| Bacteroides sp. D20 | ACPT01000052 | Oscillibacter sp. G2 | HM626173 |
| Bacteroides sp. D22 | ADCK01000151 | Oscillibacter valericigenes | NR_074793 |
| Bacteroides sp. F_4 | AB470322 | Oscillospira. guilliermondii | AB040495 |
| Bacteroides sp. NB_8 | AB117565 | Paenibacillus barcinonensis | NR_042272 |
| Bacteroides sp. WH2 | AY895180 | Paenibacillus barengoltzii | NR_042756 |
| Bacteroides sp. XB12B | AM230648 | Paenibacillus chibensis | NR_040885 |
| Bacteroides sp. XB44A | AM230649 | Paenibacillus cookii | NR_025372 |
| Bacteroides stercoris | ABFZ02000022 | Paenibacillus durus | NR_037017 |
| Bacteroides thetaiotaomicron | NR_074277 | Paenibacillus glucanolyticus | D78470 |
| Bacteroides uniformis | AB050110 | Paenibacillus lactis | NR_025739 |
| Bacteroides ureolyticus | GQ167666 | Paenibacillus lautus | NR_040882 |
| Bacteroides vulgatus | CP000139 | Paenibacillus pabuli | NR_040853 |
| Bacteroides xylanisolvens | ADKP01000087 | Paenibacillus polymyxa | NR_037006 |
| Bacteroidetes bacterium oral taxon D27 | HM099638 | Paenibacillus popilliae | NR_040888 |
| Bacteroidetes bacterium oral taxon F31 | HM099643 | Paenibacillus sp. CIP 101062 | HM212646 |
| Bacteroidetes bacterium oral taxon F44 | HM099649 | Parabacteroides distasonis | CP000140 |
| Barnesiella intestinihominis | AB370251 | Parabacteroides goldsteinii | AY974070 |
| Bifidobacteriaceae genomosp. C1 | AY278612 | Parabacteroides gordonii | AB470344 |
| Bifidobacterium adolescentis | AAXD02000018 | Parabacteroides johnsonii | ABYH01000014 |
| Bifidobacterium angulatum | ABYS02000004 | Parabacteroides merdae | EU136685 |
| Bifidobacterium animalis | CP001606 | Parabacteroides sp. D13 | ACPW01000017 |
| Bifidobacterium bifidum | ABQP01000027 | Parabacteroides sp. NS313 | JN029805 |
| Bifidobacterium breve | CP002743 | Peptococcus niger | NR_029221 |
| Bifidobacterium catenulatum | ABXY01000019 | Peptococcus sp. oral clone JM048 | AY349389 |
| Bifidobacterium dentium | CP001750 | Peptococcus sp. oral taxon 167 | GQ422727 |
| Bifidobacterium gallicum | ABXB03000004 | Peptoniphilus asaccharolyticus | D14145 |
| Bifidobacterium infantis | AY151398 | Peptoniphilus duerdenii | EU526290 |
| Bifidobacterium kashiwanohense | AB491757 | Peptoniphilus harei | NR_026358 |
| Bifidobacterium longum | ABQQ01000041 | Peptoniphilus indolicus | AY153431 |
| Bifidobacterium pseudocatenulatum | ABXX02000002 | Peptoniphilus ivorii | Y07840 |
| Bifidobacterium pseudolongum | NR_043442 | Peptoniphilus lacrimalis | ADD001000050 |
| Bifidobacterium scardovii | AJ307005 | Peptoniphilus sp. gpac007 | AM176517 |
| Bifidobacterium sp. HM2 | AB425276 | Peptoniphilus sp. gpac018A | AM176519 |
| Bifidobacterium sp. HMLN12 | JF519685 | Peptoniphilus sp. gpac077 | AM176527 |
| Bifidobacterium sp. M45 | HM626176 | Peptoniphilus sp. gpac148 | AM176535 |
| Bifidobacterium sp. MSX5B | HQ616382 | Peptoniphilus sp. JC140 | JF824803 |
| Bifidobacterium sp. TM_7 | AB218972 | Peptoniphilus sp. oral taxon 386 | ADCS01000031 |
| Bifidobacterium thermophilum | DQ340557 | Peptoniphilus sp. oral taxon 836 | AEAA01000090 |
| Bifidobacterium urinalis | AJ278695 | Peptostreptococcaceae bacterium ph1 | JN837495 |
| Blautia coccoides | AB571656 | Peptostreptococcus anaerobius | AY326462 |
| Blautia glucerasea | AB588023 | Peptostreptococcus micros | AM176538 |
| Blautia glucerasei | AB439724 | Peptostreptococcus sp. 9succ1 | X90471 |
| Blautia hansenii | ABYU02000037 | Peptostreptococcus sp. oral clone AP24 | AB175072 |
| Blautia hydrogenotrophica | ACBZ01000217 | Peptostreptococcus sp. oral clone FJ023 | AY349390 |
| Blautia luti | AB691576 | Peptostreptococcus sp. P4P 31 P3 | AY207059 |
| Blautia producta | AB600998 | Peptostreptococcus stomatis | ADGQ01000048 |
| Blautia schinkii | NR_026312 | Porphyromonadaceae bacterium NML 060648 | EF184292 |
| Blautia sp. M25 | HM626178 | Porphyromonas asaccharolytica | AENO01000048 |
| Blautia stercoris | HM626177 | Porphyromonas endodontalis | ACNN01000021 |
| Blautia wexlerae | EF036467 | Porphyromonas gingivalis | AE015924 |
| Bordetella bronchiseptica | NR_025949 | Porphyromonas levii | NR_025907 |
| Bordetella holmesii | AB683187 | Porphyromonas macacae | NR_025908 |
| Bordetella parapertussis | NR_025950 | Porphyromonas somerae | AB547667 |
| Bordetella pertussis | BX640418 | Porphyromonas sp. oral clone BB134 | AY005068 |
| Borrelia afzelii | ABCU01000001 | Porphyromonas sp. oral clone F016 | AY005069 |
| Borrelia burgdorferi | ABGI01000001 | Porphyromonas sp. oral clone P2PB 52 P1 | AY207054 |
| Borrelia crocidurae | DQ057990 | Porphyromonas sp. oral clone P4GB 100 P2 | AY207057 |
| Borrelia duttonii | NC_011229 | Porphyromonas sp. UQD 301 | EU012301 |
| Borrelia garinii | ABJV01000001 | Porphyromonas uenonis | ACLR01000152 |
| Borrelia hermsii | AY597657 | Prevotella albensis | NR_025300 |
| Borrelia hispanica | DQ057988 | Prevotella amnii | AB547670 |
| Borrelia persica | HM161645 | Prevotella bergensis | ACKS01000100 |
| Borrelia recurrentis | AF107367 | Prevotella bivia | ADFO01000096 |
| Borrelia sp. NE49 | AJ224142 | Prevotella brevis | NR_041954 |
| Borrelia spielmanii | ABKB01000002 | Prevotella buccae | ACRB01000001 |
| Borrelia turicatae | NC_008710 | Prevotella buccalis | JN867261 |
| Borrelia valaisiana | ABCY01000002 | Prevotella copri | ACBX02000014 |
| Brucella ovis | NC_009504 | Prevotella corporis | L16465 |
| Brucella sp. 83 13 | ACBQ01000040 | Prevotella dentalis | AB547678 |
| Brucella sp. BO1 | EU053207 | Prevotella denticola | CP002589 |
| Brucella suis | ACBK01000034 | Prevotella disiens | AED001000026 |
| Burkholderia ambifaria | AAUZ01000009 | Prevotella genoniosp. C1 | AY278624 |
| Burkholderia cenocepacia | AAHI01000060 | Prevotella genomosp. C2 | AY278625 |
| Burkholderia cepacia | NR_041719 | Prevotella genomosp. P7 oral clone MB2 P31 | DQ003620 |
| Burkholderia mallei | CP000547 | Prevotella genomosp. P8 oral clone MB3 P13 | DQ003622 |
| Burkholderia multivorans | NC_010086 | Prevotella genomosp. P9 oral clone MB7 G16 | DQ003633 |
| Burkholderia oklahomensis | DQ108388 | Prevotella heparinolytica | GQ422742 |
| Burkholderia pseudomallei | CP001408 | Prevotella histicola | JN867315 |
| Burkholderia rhizoxinica | HQ005410 | Prevotella intermedia | AF414829 |
| Burkholderia sp. 383 | CP000151 | Prevotella loescheii | JN867231 |
| Burkholderia xenovorans | U86373 | Prevotella maculosa | AGEK01000035 |
| Burkholderiales bacterium 1 1 47 | ADCQ01000066 | Prevotella marshii | AEEI01000070 |
| Butyrivibrio crossotus | ABWN01000012 | Prevotella melaninogenica | CP002122 |
| Butyrivibrio fibrisolvens | U41172 | Prevotella micans | AGWK01000061 |
| Chlamydia muridarum | AE002160 | Prevotella multiformis | AEWX01000054 |
| Chlamydia psittaci | NTR_036864 | Prevotella multisaccharivorax | AFJE01000016 |
| Chlamydia trachomatis | U68443 | Prevotella nanceiensis | JN867228 |
| Chlamydiales bacterium NS11 | JN606074 | Prevotella nigrescens | AFPX01000069 |
| Citrobacter amalonaticus | FR870441 | Prevotella oralis | AEPE01000021 |
| Citrobacter braakii | NR_028687 | Prevotella oris | ADDV01000091 |
| Citrobacter farmeri | AF025371 | Prevotella oulorum | L16472 |
| Citrobacter freundii | NR_028894 | Prevotella pallens | AFPY01000135 |
| Citrobacter gillenii | AF025367 | Prevotella ruminicola | CP002006 |
| Citrobacter koseri | NC_009792 | Prevotella salivae | AB108826 |
| Citrobacter murliniae | AF025369 | Prevotella sp. BI 42 | AJ581354 |
| Citrobacter rodentium | NR_074903 | Prevotella sp. CM38 | HQ610181 |
| Citrobacter sedlakii | AF025364 | Prevotella sp. ICM1 | HQ616385 |
| Citrobacter sp. 30 2 | ACDJ01000053 | Prevotella sp. ICM55 | HQ616399 |
| Citrobacter sp. KMS1 3 | GQ468398 | Prevotella sp. JCM 6330 | AB547699 |
| Citrobacter werkmanii | AF025373 | Prevotella sp. oral clone AA020 | AY005057 |
| Citrobacter youngae | ABWL0200001 1 | Prevotella sp. oral clone ASCG10 | AY923148 |
| Cloacibacillus evryensis | GQ258966 | Prevotella sp. oral clone ASCG12 | DQ272511 |
| Clostridiaceae bacterium END 2 | EF451053 | Prevotella sp. oral clone AU069 | AY005062 |
| Clostridiaceae bacterium JC13 | JF824807 | Prevotella sp. oral clone CY006 | AY005063 |
| Clostridiales bacterium 1 7 47FAA | ABQR01000074 | Prevotella sp. oral clone DA058 | AY005065 |
| Clostridiales bacterium 9400853 | HM587320 | Prevotella sp. oral clone FL019 | AY349392 |
| Clostridiales bacterium 9403326 | HM587324 | Prevotella sp. oral clone FU048 | AY349393 |
| Clostridiales bacterium oral clone P4PA 66P1 | AY207065 | Prevotella sp. oral clone FW035 | AY349394 |
| Clostridiales bacterium oral taxon 093 | GQ422712 | Prevotella sp. oral clone GI030 | AY349395 |
| Clostridiales bacterium oral taxon F32 | HM099644 | Prevotella sp. oral clone GI032 | AY349396 |
| Clostridiales bacterium ph2 | JN837487 | Prevotella sp. oral clone GI059 | AY349397 |
| Clostridiales bacterium SY8519 | AB477431 | Prevotella sp. oral clone GU027 | AY349398 |
| Clostridiales genomosp. BVAB3 | CP001850 | Prevotella sp. oral clone HF050 | AY349399 |
| Clostridiales sp. SM4_1 | FP929060 | Prevotella sp. oral clone ID019 | AY349400 |
| Clostridiales sp. SS3_4 | AY305316 | Prevotella sp. oral clone IDR CEC 0055 | AY550997 |
| Clostridiales sp. SSC_2 | FP929061 | Prevotella sp. oral clone IK053 | AY349401 |
| Clostridium acetobutylicum | NR_074511 | Prevotella sp. oral clone IK062 | AY349402 |
| Clostridium aerotolerans | X76163 | Prevotella sp. oral clone P4PB 83 P2 | AY207050 |
| Clostridium aldenense | NR_043680 | Prevotella sp. oral taxon 292 | GQ422735 |
| Clostridium aldrichii | NR_026099 | Prevotella sp. oral taxon 299 | ACWZ01000026 |
| Clostridium algidicarnis | NR_041746 | Prevotella sp. oral taxon 300 | GU409549 |
| Clostridium algidixylanolyticum | NR_028726 | Prevotella sp. oral taxon 302 | ACZK01000043 |
| Clostridium aminovalericum | NR_029245 | Prevotella sp. oral taxon 310 | GQ422737 |
| Clostridium amygdalinum | AY353957 | Prevotella sp. oral taxon 317 | ACQH01000158 |
| Clostridium argentinense | NR_029232 | Prevotella sp. oral taxon 472 | ACZS01000106 |
| Clostridium asparagiforme | ACCJ01000522 | Prevotella sp. oral taxon 781 | GQ422744 |
| Clostridium baratii | NR_029229 | Prevotella sp. oral taxon 782 | GQ422745 |
| Clostridium bartlettii | ABEZ02000012 | Prevotella sp. oral taxon F68 | HM099652 |
| Clostridium beijerinckii | NR_074434 | Prevotella sp. oral taxon G60 | GU432133 |
| Clostridium bifermentans | X73437 | Prevotella sp. oral taxon G70 | GU432179 |
| Clostridium bolteae | ABCC02000039 | Prevotella sp. oral taxon G71 | GU432180 |
| Clostridium botulinum | NC_010723 | Prevotella sp. SEQ053 | JN867222 |
| Clostridium butyricum | ABDT01000017 | Prevotella sp. SEQ065 | JN867234 |
| Clostridium cadaveris | AB542932 | Prevotella sp. SEQ072 | JN867238 |
| Clostridium carboxidivorans | FR733710 | Prevotella sp. SEQ116 | JN867246 |
| Clostridium carnis | NR_044716 | Prevotella sp. SG12 | GU561343 |
| Clostridium celatum | X77844 | Prevotella sp. sp24 | AB003384 |
| Clostridium celerecrescens | JQ246092 | Prevotella sp. sp34 | AB003385 |
| Clostridium cellulosi | NR_044624 | Prevotella stercorea | AB244774 |
| Clostridium chauvoei | EU106372 | Prevotella tannerae | ACIJ02000018 |
| Clostridium citroniae | ADLJ01000059 | Prevotella timonensis | ADEF01000012 |
| Clostridium clariflavum | NR_041235 | Prevotella veroralis | ACVA01000027 |
| Clostridium clostridiiformes | M59089 | Prevotellaceae bacterium P4P 62 P1 | AY207061 |
| Clostridium clostridioforme | NR_044715 | Propionibacteriaceae bacterium NML 02 0265 | EF599122 |
| Clostridium coccoides | EF025906 | Propionibacterium acidipropionici | NC_019395 |
| Clostridium cochlearium | NR_044717 | Propionibacterium acnes | ADJM01000010 |
| Clostridium cocleatum | NR_026495 | Propionibacterium avidum | AJ003055 |
| Clostridium colicanis | FJ957863 | Propionibacterium freudenreichii | NR_036972 |
| Clostridium colinum | NR_026151 | Propionibacterium granulosum | FJ785716 |
| Clostridium difficile | NC_013315 | Propionibacterium jensenii | NR_042269 |
| Clostridium disporicum | NR_026491 | Propionibacterium propionicum | NR_025277 |
| Clostridium estertheticum | NR_042153 | Propionibacterium sp. 434 HC2 | AFIL01000035 |
| Clostridium fallax | NR_044714 | Propionibacterium sp. H456 | AB177643 |
| Clostridium favososporum | X76749 | Propionibacterium sp. LG | AY354921 |
| Clostridium felsineum | AF270502 | Propionibacterium sp. oral taxon 192 | GQ422728 |
| Clostridium frigidicarnis | NR_024919 | Propionibacterium sp. S555a | AB264622 |
| Clostridium gasigenes | NR_024945 | Propionibacterium thoenii | NR_042270 |
| Clostridium ghonii | AB542933 | Pseudomonas aeruginosa | AABQ07000001 |
| Clostridium glycolicum | FJ384385 | Pseudomonas fluorescens | AY622220 |
| Clostridium glycyrrhizinilyticum | AB233029 | Pseudomonas gessardii | FJ943496 |
| Clostridium haemolyticum | NR_024749 | Pseudomonas mendocina | AAUL01000021 |
| Clostridium hathewayi | AY552788 | Pseudomonas monteilii | NR_024910 |
| Clostridium hiranonis | AB023970 | Pseudomonas poae | GU188951 |
| Clostridium histolyticum | HF558362 | Pseudomonas pseudoalcaligenes | NR_037000 |
| Clostridium hylemonae | AB023973 | Pseudomonas putida | AF094741 |
| Clostridium indolis | AF028351 | Pseudomonas sp. 2 1 26 | ACWU01000257 |
| Clostridium innocuum | M23732 | Pseudomonas sp. G1229 | DQ910482 |
| Clostridium irregulare | NR_029249 | Pseudomonas sp. NP522b | EU723211 |
| Clostridium isatidis | NR_026347 | Pseudomonas stutzeri | AM905854 |
| Clostridium kluyveri | NR_074165 | Pseudomonas tolaasii | AF320988 |
| Clostridium lactatifermentans | NR_025651 | Pseudomonas viridiflava | NR_042764 |
| Clostridium lavalense | EF564277 | Ralstonia pickettii | NC_010682 |
| Clostridium leptum | AJ305238 | Ralstonia sp. 5 7 47FAA | ACUF01000076 |
| Clostridium limosum | FR870444 | Roseburia cecicola | GU233441 |
| Clostridium magnum | X77835 | Roseburia faecalis | AY804149 |
| Clostridium malenominatum | FR749893 | Roseburia faecis | AY305310 |
| Clostridium mayombei | FR733682 | Roseburia hominis | AJ270482 |
| Clostridium methylpentosum | ACEC01000059 | Roseburia intestinalis | FP929050 |
| Clostridium nexile | X73443 | Roseburia inulinivorans | AJ270473 |
| Clostridium novyi | NR_074343 | Roseburia sp. 11SE37 | FM954975 |
| Clostridium orbiscindens | Y18187 | Roseburia sp. 11SE38 | FM954976 |
| Clostridium oroticum | FR749922 | Rothia aeria | DQ673320 |
| Clostridium paraputrificum | AB536771 | Rothia dentocariosa | ADDW01000024 |
| Clostridium perfringens | ABDW01000023 | Rothia mucilaginosa | ACVO01000020 |
| Clostridium phytofermentans | NR_074652 | Rothia nasimurium | NR_025310 |
| Clostridium piliforme | D14639 | Rothia sp. oral taxon 188 | GU470892 |
| Clostridium putrefaciens | NR_024995 | Ruminobacter amylophilus | NR_026450 |
| Clostridium quinii | NR_026149 | Ruminococcaceae bacterium D16 | ADDX01000083 |
| Clostridium ramosum | M23731 | Ruminococcus albus | AY445600 |
| Clostridium rectum | NR_029271 | Ruminococcus bromii | EU266549 |
| Clostridium saccharogumia | DQ100445 | Ruminococcus callidus | NR_029160 |
| Clostridium saccharolyticum | CP002109 | Ruminococcus champanellensis | FP929052 |
| Clostridium sardiniense | NR_041006 | Ruminococcus flavefaciens | NR_025931 |
| Clostridium sartagoforme | NR_026490 | Ruminococcus gnavus | X94967 |
| Clostridium scindens | AF262238 | Ruminococcus hansenii | M59114 |
| Clostridium septicum | NR_026020 | Ruminococcus lactaris | ABOU02000049 |
| Clostridium sordellii | AB448946 | Ruminococcus obeum | AY169419 |
| Clostridium sp. 7 2 43FAA | ACDK01000101 | Ruminococcus sp. 18P13 | AJ515913 |
| Clostridium sp. D5 | ADBG01000142 | Ruminococcus sp. 5 1 39BFAA | ACII01000172 |
| Clostridium sp. HGF2 | AENW01000022 | Ruminococcus sp. 9SE51 | FM954974 |
| Clostridium sp. HPB 46 | AY862516 | Ruminococcus sp. ID8 | AY960564 |
| Clostridium sp. JC122 | CAEV01000127 | Ruminococcus sp. K 1 | AB222208 |
| Clostridium sp. L2 50 | AAYW02000018 | Ruminococcus torques | AAVP02000002 |
| Clostridium sp. LMG 16094 | X95274 | Salmonella, bongori | NR_041699 |
| Clostridium sp. M62 1 | ACFX02000046 | Salmonella enterica | NC_011149 |
| Clostridium sp. MLG055 | AF304435 | Salmonella, enterica | NC_011205 |
| Clostridium sp. MT4 E | FJ159523 | Salmonella enterica | DQ344532 |
| Clostridium sp. NMBHI 1 | JN093130 | Salmonella, enterica | ABEH02000004 |
| Clostridium sp. NML 04A032 | EU815224 | Salmonella enterica | ABAK02000001 |
| Clostridium sp. SS2 1 | ABGC03000041 | Salmonella, enterica | NC_011080 |
| Clostridium sp. SY8519 | AP012212 | Salmonella enterica | EU118094 |
| Clostridium sp. TM 40 | AB249652 | Salmonella enterica | NC_011094 |
| Clostridium sp. YIT 12069 | AB491207 | Salmonella enterica | AE014613 |
| Clostridium sp. YIT 12070 | AB491208 | Salmonella enterica | ABFH02000001 |
| Clostridium sphenoides | X73449 | Salmonella enterica | ABEM01000001 |
| Clostridium spiroforme | X73441 | Salmonella enterica | ABAM02000001 |
| Clostridium sporogenes | ABKW02000003 | Salmonella typhimurium | DQ344533 |
| Clostridium sporosphaeroides | NR_044835 | Salmonella typhimurium | AF170176 |
| Clostridium stercorarium | NR_025100 | Selenomonas artemidis | HM596274 |
| Clostridium sticklandii | L04167 | Selenomonas dianae | GQ422719 |
| Clostridium straminisolvens | NR_024829 | Selenomonas flueggei | AF287803 |
| Clostridium subterminale | NR_041795 | Selenomonas genomosp. C1 | AY278627 |
| Clostridium sulfidigenes | NR_044161 | Selenomonas genomosp. C2 | AY278628 |
| Clostridium symbiosum | ADLQ01000114 | Selenomonas genomosp. P5 | AY341820 |
| Clostridium tertium | Y18174 | Selenomonas genomosp. P6 oral clone MB3 C41 | DQ003636 |
| Clostridium tetani | NC_004557 | Selenomonas genomosp. P7 oral clone MB5 C08 | DQ003627 |
| Clostridium thermocellum | NR_074629 | Selenomonas genomosp. P8 oral clone MB5 P06 | DQ003628 |
| Clostridium tyrobutyricum | NR_044718 | Selenomonas infelix | AF287802 |
| Clostridium viride | NR_026204 | Selenomonas noxia | GU470909 |
| Clostridium xylatiolyticiini | NR_037068 | Selenomonas ruminantium | NR_075026 |
| Collinsella aerofaciens | AAVN02000007 | Selenomonas sp. FOBRC9 | HQ616378 |
| Collinsella intestinalis | ABXH02000037 | Selenomonas sp. oral clone FT050 | AY349403 |
| Collinsella stercoris | ABXJ01000150 | Selenomonas sp. oral clone GI064 | AY349404 |
| Collinsella tanakaei | AB490807 | Selenomonas sp. oral clone GT010 | AY349405 |
| Coprobacillus cateniformis | AB030218 | Selenomonas sp. oral clone HU051 | AY349406 |
| Coprobacillus sp. 29_1 | ADKX01000057 | Selenomonas sp. oral clone IK004 | AY349407 |
| Coprobacillus sp. D7 | ACDT01000199 | Selenomonas sp. oral clone IQ048 | AY349408 |
| Coprococcus catus | EU266552 | Selenomonas sp. oral clone JI021 | AY349409 |
| Coprococcus comes | ABVR01000038 | Selenomonas sp. oral clone JS031 | AY349410 |
| Coprococcus eutactus | EF031543 | Selenomonas sp. oral clone OH4A | AY947498 |
| Coprococcus sp. ART55_1 | AY350746 | Selenomonas sp. oral clone P2PA 80 P4 | AY207052 |
| Dialister invisus | ACIM02000001 | Selenomonas sp. oral taxon 137 | AENV01000007 |
| Dialister micraerophilus | AFBB01000028 | Selenomonas sp. oral taxon 149 | AEFJ01000007 |
| Dialister microaerophilus | AENT01000008 | Selenomonas sputigena | ACKP02000033 |
| Dialister pneumosintes | HM596297 | Serratia fonticola | NR_025339 |
| Dialister propionicifaciens | NR_043231 | Serratia liquefaciens | NR_042062 |
| Dialister sp. oral taxon 502 | GQ422739 | Serratia. marcescens | GU826157 |
| Dialister succinatiphilus | AB370249 | Serratia odorifera | ADBY01000001 |
| Dorea formicigenerans | AAXA02000006 | Serratia proteamaculans | AAUN01000015 |
| Dorea longicatena | AJ132842 | Shigella boydii | AAKA01000007 |
| Enhydrobacter aerosaccus | ACYI01000081 | Shigella dysenteriae | NC_007606 |
| Enterobacter aerogenes | AJ251468 | Shigella flexneri | AE005674 |
| Enterobacter asburiae | NR_024640 | Shigella sonnei | NC_007384 |
| Enterobacter cancerogenus | Z96078 | Sphingobacterium faecium | NR_025537 |
| Enterobacter cloacae | FP929040 | Sphingobacterium mizutaii | JF708889 |
| Enterobacter cowanii | NR_025566 | Sphingobacterium multivorum | NR_040953 |
| Enterobacter hormaechei | AFHR01000079 | Sphingobacterium spiritivorum | ACHA02000013 |
| Enterobacter sp. 247BMC | HQ122932 | Sphingomonas echinoides | NR_024700 |
| Enterobacter sp. 638 | NR_074777 | Sphingomonas sp. oral clone FI012 | AY349411 |
| Enterobacter sp. JC163 | JN657217 | Sphingomonas sp. oral clone FZ016 | AY349412 |
| Enterobacter sp. SCSS | HM007811 | Sphingomonas sp. oral taxon A09 | HM099639 |
| Enterobacter sp. TSE38 | HM156134 | Sphingomonas sp. oral taxon. F71 | HM099645 |
| Enterobacteriaceae bacterium 9 2 54FAA | ADCU01000033 | Staphylococcaceae bacterium NML 92 0017 | AY841362 |
| Enterobacteriaceae bacterium CF01Ent_1 | AJ489826 | Staphylococcus aureus | CP002643 |
| Enterobacteriaceae bacterium Smarlab 3302238 | AY538694 | Staphylococcus auricularis | JQ624774 |
| Enterococcus avium | AF133535 | Staphylococcus capitis | ACFR01000029 |
| Enterococcus caccae | AY943820 | Staphylococcus caprae | ACRH01000033 |
| Enterococcus casseliflavus | AEWT01000047 | Staphylococcus carnosus | NR_075003 |
| Enterococcus durans | AJ276354 | Staphylococcus cohnii | JN175375 |
| Enterococcus faecalis | AE016830 | Staphylococcus condimenti | NR_029345 |
| Enterococcus faecium | AM157434 | Staphylococcus epidermidis | ACHE01000056 |
| Enterococcus gallinarum | AB269767 | Staphylococcus equorum | NR_027520 |
| Enterococcus gilvus | AY033814 | Staphylococcus fleurettii | NR_041326 |
| Enterococcus hawaiiensis | AY321377 | Staphylococcus haemolyticus | NC_007168 |
| Enterococcus hirae | AF061011 | Staphylococcus hominis | AM157418 |
| Enterococcus italicus | AEPV01000109 | Staphylococcus lugdunensis | AEQA01000024 |
| Enterococcus mundtii | NR 024906 | Staphylococcus pasteuri | FJ189773 |
| Enterococcus raffinosus | FN600541 | Staphylococcus pseudintermedius | CP002439 |
| Enterococcus sp. BV2CASA2 | JN809766 | Staphylococcus saccharolyticus | NR_029158 |
| Enterococcus sp. CCRI 16620 | GU457263 | Staphylococcus saprophyticus | NC_007350 |
| Enterococcus sp. F95 | FJ463817 | Staphylococcus sciuri | NR_025520 |
| Enterococcus sp. RfL6 | AJ133478 | Staphylococcus sp. clone bottae7 | AF467424 |
| Enterococcus thailandicus | AY321376 | Staphylococcus sp. H292 | AB177642 |
| Erysipelotrichaceae bacterium 3_1_53 | ACTJ01000113 | Staphylococcus sp. H780 | AB177644 |
| Erysipelotrichaceae bacterium 5_2_54FAA | ACZW01000054 | Staphylococcus succinus | NR_028667 |
| Escherichia albertii | ABKX01000012 | Staphylococcus vitulinus | NR_024670 |
| Escherichia coli | NC_008563 | Staphylococcus warneri | ACPZ01000009 |
| Escherichia fergusonii | CU928158 | Staphylococcus xylosus | AY395016 |
| Escherichia hermannii | HQ407266 | Streptobacillus moniliformis | NR_027615 |
| Escherichia sp. 1 1 43 | ACID01000033 | Streptococcus agalactiae | AAJO01000130 |
| Escherichia sp. 4_1_40B | ACDM02000056 | Streptococcus alactolyticus | NR_041781 |
| Escherichia sp. B4 | EU722735 | Streptococcus anginosus | AECT01000011 |
| Escherichia vulneris | NR_041927 | Streptococcus australis | AEQR01000024 |
| Eubacteriaceae bacterium P4P 50 P4 | AY207060 | Streptococcus bovis | AEEL01000030 |
| Eubacterium barkeri | NR_044661 | Streptococcus canis | AJ413203 |
| Eubacterium biforme | ABYT01000002 | Streptococcus constellatus | AY277942 |
| Eubacterium brachy | U13038 | Streptococcus cristatus | AEVC01000028 |
| Eubacterium budayi | NR_024682 | Streptococcus downei | AEKN01000002 |
| Eubacterium callanderi | NR_026330 | Streptococcus dysgalactiae | AP010935 |
| Eubacterium cellulosolvens | AY178842 | Streptococcus equi | CP001129 |
| Eubacterium contortum | FR749946 | Streptococcus equinus | AEVB01000043 |
| Eubacterium coprostanoligenes | HM037995 | Streptococcus gallolyticus | FR824043 |
| Eubacterium cylindroides | FP929041 | Streptococcus genomosp. C1 | AY278629 |
| Eubacterium desmolans | NR_044644 | Streptococcus genomosp. C2 | AY278630 |
| Eubacterium dolichum | L34682 | Streptococcus genomosp. C3 | AY278631 |
| Eubacterium eligens | CP001104 | Streptococcus genomosp. C4 | AY278632 |
| Eubacterium fissicatena | FR749935 | Streptococcus genomosp. C5 | AY278633 |
| Eubacterium hadrum | FR749933 | Streptococcus genomosp. C6 | AY278634 |
| Eubacterium hallii | L34621 | Streptococcus genomosp. C7 | AY278635 |
| Eubacterium infirmum | U13039 | Streptococcus genomosp. C8 | AY278609 |
| Eubacterium limosum | CP002273 | Streptococcus gordonii | NC_009785 |
| Eubacterium moniliforme | HF558373 | Streptococcus infantarius | ABJK02000017 |
| Eubacterium multiforme | NR_024683 | Streptococcus infantis | AFNN01000024 |
| Eubacterium nitritogenes | NR_024684 | Streptococcus intermedius | NR_028736 |
| Eubacterium nodatum | U13041 | Streptococcus lutetiensis | NR_037096 |
| Eubacterium ramulus | AJ011522 | Streptococcus massiliensis | AY769997 |
| Eubacterium rectale | FP929042 | Streptococcus milleri | X81023 |
| Eubacterium ruminantium | NR_024661 | Streptococcus mitis | AM157420 |
| Eubacterium saburreum | AB525414 | Streptococcus mutans | AP010655 |
| Eubacterium saphenum | NR_026031 | Streptococcus oligofermentans | AY099095 |
| Eubacterium siraeum | ABCA03000054 | Streptococcus oralis | ADMV01000001 |
| Eubacterium sp. 3_1_31 | ACTL01000045 | Streptococcus parasanguinis | AEKM01000012 |
| Eubacterium sp. AS15b | HQ616364 | Streptococcus pasteurianus | AP012054 |
| Eubacterium sp. OBRC9 | HQ616354 | Streptococcus peroris | AEVF01000016 |
| Eubacterium sp. oral clone GI038 | AY349374 | Streptococcus pneumoniae | AE008537 |
| Eubacterium sp.oral clone IR009 | AY349376 | Streptococcus porcinus | EF121439 |
| Eubacterium sp. oral clone JH012 | AY349373 | Streptococcus pseudopneumoniae | FJ827123 |
| Eubacterium sp.oral clone JI012 | AY349379 | Streptococcus pseudoporcinus | AENS01000003 |
| Eubacterium sp.oral clone JN088 | AY349377 | Streptococcus pyogenes | AE006496 |
| Eubacterium sp.oral clone JS001 | AY349378 | Streptococcus ratti | X58304 |
| Eubacterium sp. oral clone OH3A | AY947497 | Streptococcus salivarius | AGBV01000001 |
| Eubacterium sp. WAL 14571 | FJ687606 | Streptococcus sanguinis | NR_074974 |
| Eubacterium tenue | M59118 | Streptococcus sinensis | AF432857 |
| Eubacterium tortuosum | NR_044648 | Streptococcus sp. 16362 | JN590019 |
| Eubacterium ventriosum | L34421 | Streptococcus sp. 2 1 36FAA | ACOI01000028 |
| Eubacterium xylanophilum | L34628 | Streptococcus sp. 2285 97 | AJ131965 |
| Eubacterium yurii | AEES01000073 | Streptococcus sp. 69130 | X78825 |
| Fusobacterium canifelinum | AY162222 | Streptococcus sp. AC15 | HQ616356 |
| Fusobacterium genomosp. C1 | AY278616 | Streptococcus sp. ACS2 | HQ616360 |
| Fusobacterium genomosp. C2 | AY278617 | Streptococcus sp. AS20 | HQ616366 |
| Fusobacterium gonidiaformans | ACET01000043 | Streptococcus sp. BS35a | HQ616369 |
| Fusobacterium mortiferum | ACDB02000034 | Streptococcus sp. C150 | ACRI01000045 |
| Fusobacterium naviforme | HQ223106 | Streptococcus sp. CM6 | HQ616372 |
| Fusobacterium necrogenes | X55408 | Streptococcus sp. CM7 | HQ616373 |
| Fusobacterium necrophorum | AM905356 | Streptococcus sp. ICM10 | HQ616389 |
| Fusobacterium nucleatum | ADVK01000034 | Streptococcus sp. ICM12 | HQ616390 |
| Fusobacterium periodonticum | ACJY01000002 | Streptococcus sp. ICM2 | HQ616386 |
| Fusobacterium russii | NR_044687 | Streptococcus sp. ICM4 | HQ616387 |
| Fusobacterium sp. 1 1 41FAA | ADGG01000053 | Streptococcus sp. ICM45 | HQ616394 |
| Fusobacterium sp. 11 3 2 | ACUO01000052 | Streptococcus sp. M143 | ACRK01000025 |
| Fusobacterium sp. 12 1B | AGWJ01000070 | Streptococcus sp. M334 | ACRL01000052 |
| Fusobacterium sp. 2_1_31 | ACDC02000018 | Streptococcus sp. OBRC6 | HQ616352 |
| Fusobacterium sp. 3_1_27 | ADGF01000045 | Streptococcus sp. oral clone ASB02 | AY923121 |
| Fusobacterium sp. 3_1_33 | ACQE01000178 | Streptococcus sp. oral clone ASCA03 | DQ272504 |
| Fusobacterium sp. 3 1 36A2 | ACPU01000044 | Streptococcus sp. oral clone ASCA04 | AY923116 |
| Fusobacterium sp. 3_1_5R | ACDD01000078 | Streptococcus sp. oral clone ASCA09 | AY923119 |
| Fusobacterium sp. AC18 | HQ616357 | Streptococcus sp. oral clone ASCB04 | AY923123 |
| Fusobacterium sp. ACB2 | HQ616358 | Streptococcus sp. oral clone ASCB06 | AY923124 |
| Fusobacterium sp. AS2 | HQ616361 | Streptococcus sp. oral clone ASCC04 | AY923127 |
| Fusobacterium sp. CM1 | HQ616371 | Streptococcus sp. oral clone ASCC05 | AY923128 |
| Fusobacterium sp. CM21 | HQ616375 | Streptococcus sp. oral clone ASCC12 | DQ272507 |
| Fusobacterium sp. CM22 | HQ616376 | Streptococcus sp. oral clone ASCD01 | AY923129 |
| Fusobacterium sp. D12 | ACDG02000036 | Streptococcus sp. oral clone ASCD09 | AY923130 |
| Fusobacterium sp. oral clone ASCF06 | AY923141 | Streptococcus sp. oral clone ASCD10 | DQ272509 |
| Fusobacterium sp. oral clone ASCF11 | AY953256 | Streptococcus sp. oral clone ASCE03 | AY923134 |
| Fusobacterium ulcerans | ACDH01000090 | Streptococcus sp. oral clone ASCE04 | AY953253 |
| Fusobacterium varium | AClE01000009 | Streptococcus sp. oral clone ASCE05 | DQ272510 |
| Gemella haemolysans | ACDZ02000012 | Streptococcus sp. oral clone ASCE06 | AY923135 |
| Gemella morbillorum | NR_025904 | Streptococcus sp. oral clone ASCE09 | AY923136 |
| Gemella morbillorum | ACRX01000010 | Streptococcus sp. oral clone ASCE10 | AY923137 |
| Gemella sanguinis | ACRY01000057 | Streptococcus sp. oral clone ASCE12 | AY923138 |
| Gemella sp. oral clone ASCE02 | AY923133 | Streptococcus sp. oral clone ASCF05 | AY923140 |
| Gemella sp. oral clone ASCF04 | AY923139 | Streptococcus sp. oral clone ASCF07 | AY953255 |
| Gemella sp. oral clone ASCF12 | AY923143 | Streptococcus sp. oral clone ASCF09 | AY923142 |
| Gemella sp. WAL 1945J | EU427463 | Streptococcus sp. oral clone ASCG04 | AY923145 |
| Klebsiella oxytoca | AY292871 | Streptococcus sp. oral clone BW009 | AY005 042 |
| Klebsiella pneumoniae | CP000647 | Streptococcus sp. oral clone CH016 | AY 005044 |
| Klebsiella sp. AS10 | HQ616362 | Streptococcus sp. oral clone GK051 | AY349413 |
| Klebsiella sp. Co9935 | DQ068764 | Streptococcus sp. oral clone GM006 | AY349414 |
| Klebsiella sp. enrichment culture clone SRC_DSD25 | HM195210 | Streptococcus sp. oral clone P2PA 41 P2 | AY207051 |
| Klebsiella sp. OBRC7 | HQ616353 | Streptococcus sp. oral clone P4PA 30 P4 | AY207064 |
| Klebsiella sp. SP_BA | FJ999767 | Streptococcus sp. oral taxon 071 | AEEP01000019 |
| Klebsiella sp. SRC_DSD1 | GU797254 | Streptococcus sp. oral taxon G59 | GU432132 |
| Klebsiella sp. SRC_DSD11 | GU797263 | Streptococcus sp. oral taxon G62 | GU432146 |
| Klebsiella sp. SRC_DSD12 | GU797264 | Streptococcus sp. oral taxon G63 | GU432150 |
| Klebsiella sp. SRC_DSD15 | GU797267 | Streptococcus sp. SHV515 | Y07601 |
| Klebsiella sp. SRC DSD2 | GU797253 | Streptococcus suis | FM252032 |
| Klebsiella sp. SRC_DSD6 | GU797258 | Streptococcus thermophilus | CP000419 |
| Klebsiella variicola | CP001891 | Streptococcus uberis | HQ391900 |
| Lachnobacterium bovis | GU324407 | Streptococcus urinalis | DQ303194 |
| Lachnospira multipara. | FR733699 | Streptococcus vestibularis | AEKO01000008 |
| Lachnospira pectinoschiza | L14675 | Streptococcus viridans | AF076036 |
| Lachnospiraceae bacterium 1 1 57FAA | ACTM01000065 | Sutterella morbirenis | AJ832129 |
| Lachnospiraceae bacterium 1 4 56FAA | ACTN01000028 | Sutterella parvirubra. | AB300989 |
| Lachnospiraceae bacterium 2 1 46F4A | ADLB01000035 | Sutterella sanguinus | AJ748647 |
| Lachnospiraceae bacterium 2 1 58FAA | ACTO0100005 | Sutterella sp. YIT 12072 | AB491210 |
| Lachnospiraceae bacterium 3 1 57FAA CT1 | ACTP01000124 | Sutterella stercoricanis | NR_025600 |
| Lachnospiraceae bacterium 4 137FAA | ADCR01000030 | Sutterella wadsworthensis | ADMF01000048 |
| Lachnospiraceae bacterium 5157FAA | ACTR01000020 | Synergistes genomosp. C1 | AY278615 |
| Lachnospiraceae bacterium 5 1 63FAA | ACTS01000081 | Synergistes sp. RMA 14551 | DQ412722 |
| Lachnospiraceae bacterium 6 1 63FAA | ACTV01000014 | Synergistetes bacterium ADV897 | GQ258968 |
| Lachnospiraceae bacterium 81 57FAA | ACWQ01000079 | Synergistetes bacterium LBVCM1157 | GQ258969 |
| Lachnospiraceae bacterium 9 1 43BFAA | ACTX01000023 | Synergistetes bacterium oral taxon 362 | GU410752 |
| Lachnospiraceae bacterium A4 | DQ789118 | Synergistetes bacterium oral taxon D48 | GU43 0992 |
| Lachnospiraceae bacterium DJFVP30 | EU728771 | Turicibacter sanguinis | AF349724 |
| Lachnospiraceae bacterium ICM62 | HQ616401 | Veillonella atypica | AEDS01000059 |
| Lachnospiraceae bacterium MSX33 | HQ616384 | Veillonella dispar | ACIK02000021 |
| Lachnospiraceae bacterium oral taxon 107 | ADDS01000069 | Veillonella genomosp. P1 oral clone MB5 P17 | DQ003631 |
| Lachnospiraceae bacterium oral taxon F15 | HM099641 | Veillonella montpellierensis | AF473836 |
| Lachnospiraceae genomosp. C1 | AY278618 | Veillonella parvula | ADFU01000009 |
| Lactobacillus acidipiscis | NR_ 024718 | Veillonella sp. 3 1 44 | ADCV01000019 |
| Lactobacillus acidophilus | CP000033 | Veillonella sp. 6 1 27 | ADCW01000016 |
| Lactobacillus alimentarius | NR_ 044701 | Veillonella sp. ACP1 | HQ616359 |
| Lactobacillus amylolyticus | ADNY01000006 | Veillonella sp. AS16 | HQ616365 |
| Lactobacillus amylovorus | CP002338 | Veillonella sp. BS32b | HQ616368 |
| Lactobacillus antri | ACLL01000037 | Veillonella sp. ICM51a | HQ616396 |
| Lactobacillus brevis | EU194349 | Veillonella sp. MSA12 | HQ616381 |
| Lactobacillus buchneri | ACGH01000101 | Veillonella sp. NVG 100cf | EF108443 |
| Lactobacillus casei | CP000423 | Veillonella sp. OK11 | JN695650 |
| Lactobacillus catenaformis | M23729 | Veillonella sp. oral clone ASCA08 | AY923118 |
| Lactobacillus coleohominis | ACOH01000030 | Veillonella sp. oral clone ASCB03 | AY923122 |
| Lactobacillus coryniformis | NR_044705 | Veillonella sp. oral clone ASCG01 | AY923144 |
| Lactobacillus crispatus | ACOG01000151 | Veillonella sp. oral clone ASCG02 | AY953257 |
| Lactobacillus curvatus | NR_042437 | Veillonella sp. oral clone OH1A | AY947495 |
| Lactobacillus delbrueckii | CP002341 | Veillonella sp. oral taxon 158 | AENU01000007 |
| Lactobacillus dextrinicus | NR_036861 | Veillonellaceae bacterium oral taxon 131 | GU402916 |
| Lactobacillus farciminis | NR_044707 | Veillonellaceae bacterium oral taxon 155 | GU470897 |
| Lactobacillus fermentum | CP002033 | Vibrio cholerae | AAUR01000095 |
| Lactobacillus gasseri | ACOZ01000018 | Vibrio fluvialis | X76335 |
| Lactobacillus gastricus | AICN01000060 | Vibrio furnissii | CP002377 |
| Lactobacillus genomosp. C1 | AY278619 | Vibrio mimicus | ADAF01000001 |
| Lactobacillus genomosp. C2 | AY278620 | Vibrio parahaemolyticus | AAWQ01000116 |
| Lactobacillus helveticus | ACLM01000202 | Vibrio sp. RC341 | ACZT01000024 |
| Lactobacillus hilgardii | ACGP01000200 | Vibrio vulnificus | AE016796 |
| Lactobacillus hominis | FR681902. | Yersinia aldovae | AJ871363 |
| Lactobacillus iners | AEKJ01000002. | Yersinia aleksiciae | AJ62759 |
| Lactobacillus jensenii | ACQD01000066 | Yersinia bercovieri | AF366377 |
| Lactobacillus johnsonii | AE017198 | Yersinia enterocolitica | FR729477 |
| Lactobacillus kalixensis | NR_029083 | Yersinia frederiksenii | AF366379 |
| Lactobacillus kefiranofaciens | NR_042440 | Yersinia intermedia | AF366380 |
| Lactobacillus kefiri | NR_042230 | Yersinia kristensenii | ACCA01000078 |
| Lactobacillus kimchii | NR_025045 | Yersinia mollaretii | NR_027546 |
| Lactobacillus leichmannii | JX986966 | Yersinia pestis | AE013632 |
| Lactobacillus mucosae | FR693800 | Yersinia pseudotuberculosis | NC_009708 |
| | | Yersinia rohdei | ACCD01000071 |

TABLE 3

Exemplary Bacterial Strains

| | |
|---|---|
| Strain | Deposit Number |
| Parabacteroides goldsteinii | PTA-126574 |
| Bifidobacterium animalis ssp. lactis Strain A | PTA-125097 |
| Blautia Massiliensis Strain A | PTA-125134 |
| Prevotella Strain B | NRRL accession Number B 50329 |
| Prevotella. Histicola | PTA-126140 |
| Blautia Strain A | PTA-125346 |
| Lactococcus lactis cremoris Strain A | PTA-125368 |
| Lactobacillus salivarius | PTA-125893 |
| Ruminococcus gnavus strain | PTA-125706 |
| Tyzzerella nexilis strain | PTA-125707 |
| Paraclostridium benzoelyticum | PTA-125894 |
| Ruminococcus gnavus (also referred to as Mediterraneibacter gnavus) | PTA-126695 |
| Veillonella parvula | PTA-125710 |
| Veillonella atypica Strain A | PTA-125709 |
| Veillonella atypica Strain B | PTA-125711 |
| Veillonella parvula Strain A | PTA-125691 |
| Veillonella parvula Strain B | PTA-125711 |
| Veillonella tobetsuensis Strain A | PTA-125708 |
| Agathobaculum sp. | PTA-125892 |
| Turicibacter sanguinis | PTA-125889 |
| Klebsiella quasipneumoniae subsp. similipneumoniae | PTA-125891 |
| Klebsiella oxytoca | PTA-125890 |
| Megasphaera Sp. Strain A | PTA-126770 |
| Megasphaera Sp. | PTA-126837 |
| Harryflintia acetispora | PTA-126694 |
| Fournierella massiliensis | PTA-126696 |

### Modified Bacteria and mEVs

In some aspects, the bacteria and/or mEVs (such as smEVs and/or pmEVs) described herein are modified such that they comprise, are linked to, and/or are bound by a therapeutic moiety.

In some embodiments, the therapeutic moiety is a cancer-specific moiety . In some embodiments, the cancer-specific moiety has binding specificity for a cancer cell (e.g., has binding specificity for a cancer-specific antigen). In some embodiments, the cancer-specific moiety comprises an antibody or antigen binding fragment thereof. In some embodiments, the cancer-specific moiety comprises a T cell receptor or a chimeric antigen receptor (CAR). In some embodiments, the cancer-specific moiety comprises a ligand for a receptor expressed on the surface of a cancer cell or a receptor-binding fragment thereof. In some embodiments, the cancer-specific moiety is a bipartite fusion protein that has two parts: a first part that binds to and/or is linked to the bacterium and a second part that is capable of binding to a cancer cell (e.g., by having binding specificity for a cancer-specific antigen). In some embodiments, the first part is a fragment of or a full-length peptidoglycan recognition protein, such as PGRP. In some embodiments the first part has binding specificity for the mEV (e.g., by having binding specificity for a bacterial antigen). In some embodiments, the first and/or second part comprises an antibody or antigen binding fragment thereof. In some embodiments, the first and/or second part comprises a T cell receptor or a chimeric antigen receptor (CAR). In some embodiments, the first and/or second part comprises a ligand for a receptor expressed on the surface of a cancer cell or a receptor-binding fragment thereof. In certain embodiments, co-administration of the cancer-specific moiety with the pharmaceutical agent (either in combination or in separate administrations) increases the targeting of the pharmaceutical agent to the cancer cells.

In some embodiments, the bacteria and/or mEVs described herein can be modified such that they comprise, are linked to, and/or are bound by a magnetic and/or paramagnetic moiety (e.g., a magnetic bead). In some embodiments, the magnetic and/or paramagnetic moiety is comprised by and/or directly linked to the bacteria. In some embodiments, the magnetic and/or paramagnetic moiety is linked to and/or a part of a bacteria-or an mEV-binding moiety that binds to the bacteria or mEV. In some embodiments, the bacteria- or mEV-binding moiety is a fragment of or a full-length peptidoglycan recognition protein, such as PGRP. In some embodiments the bacteria- or mEV-binding moiety has binding specificity for the bacteria or mEV (e.g., by having binding specificity for a bacterial antigen). In some embodiments, the bacteria- or mEV-binding moiety comprises an antibody or antigen binding fragment thereof. In some embodiments, the bacteria- or mEV-binding moiety comprises a T cell receptor or a chimeric antigen receptor (CAR). In some embodiments, the bacteria- or mEV-binding moiety comprises a ligand for a receptor expressed on the surface of a cancer cell or a receptor-binding fragment thereof. In certain embodiments, co-administration of the magnetic and/or paramagnetic moiety with the bacteria or mEVs (either together or in separate administrations) can be used to increase the targeting of the mEVs (e.g., to cancer cells and/or a part of a subject where cancer cells are present.

### Production of Processed Microbial Extracellular Vesicles (pmEVs)

In certain aspects, the pmEVs described herein can be prepared using any method known in the art.

In some embodiments, the pmEVs are prepared without a pmEV purification step. For example, in some embodiments, bacteria from which the pinEVs described herein are released are killed using a method that leaves the bacterial pmEVs intact, and the resulting bacterial components, including the pmEVs, are used in the methods and compositions described herein. In some embodiments, the bacteria are killed using an antibiotic (e.g., using an antibiotic described herein). In some embodiments, the bacteria are killed using UV irradiation.

In some embodiments, the pmEVs described herein are purified from one or more other bacterial components. Methods for purifying pmEVs from bacteria (and optionally, other bacterial components) are known in the art. In some embodiments, pmEVs are prepared from bacterial cultures using methods described in Thein, et al. (J. Proteome Res. 9(12):6135-6147 (2010)) or Sandrini, et al. (Bio-protocol 4(21): el28 1′ (2014)), each of which is hereby incorporated by reference in its entirety. In some embodiments, the bacteria are cultured to high optical density and then centrifuged to pellet bacteria (e.g., at 10,000- 15,000 × g for 10- 15 min at room temperature or 4° C.). In some embodiments, the supernatants are discarded and cell pellets are frozen at -80° C. In some embodiments, cell pellets are thawed on ice and resuspended in 100 mM Tris-HCl, pH 7.5 supplemented with 1 mg/mL DNase I. In some embodiments, cells are lysed using an Emulsiflex C-3 (Avestin, Inc.) under conditions recommended by the manufacturer. In some embodiments, debris and unlysed cells are pelleted by centrifugation at 10,000 × g for 15 min at 4° C. In some embodiments, supernatants are then centrifuged at 120,000 × g for 1 hour at 4° C., In some embodiments, pellets are resuspended in ice-cold 100 mM sodium carbonate, pH 11, incubated with agitation for 1 hr at 4° C., and then centrifuged at 120,000 × g for 1 hour at 4° C. In some embodiments, pellets are resuspended in 100 mM Tris-HCl, pH 7.5, re-centrifuged at 120,000 × g for 20 mm at 4° C., and then resuspended in 0.1 M Tris-HCl, pH 7.5 or in PBS. In some embodiments, samples are stored at -20° C.

In certain aspects, pmEVs are obtained by methods adapted from Sandrini et al, 2014. In some embodiments, bacterial cultures are centrifuged at 10,000-15,500 × g for 10-15 min at room temp or at 4° C. In some embodiments, cell pellets are frozen at -80° C. and supernatants are discarded. In some embodiments, cell pellets are thawed on ice and resuspended in 10 mM Tris-HCl, pH 8.0, 1 mM ED TA supplemented with 0.1 mg/mL lysozyme. In some embodiments, samples are incubated with mixing at room temp or at 37° C. for 30 min. In some embodiments, samples are re-frozen at -80° C. and thawed again on ice. In some embodiments, DNase I is added to a final concentration of 1.6 mg/mL and MgC12 to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000 × g for 15 min. at 4° C. In some embodiments, supernatants are then centrifuged at 110,000 × g for 15 mm at 4° C. In some embodiments, pellets are resuspended in 10 mM Tris-HCl, pH 8.0, 2% Triton X-100 and incubated 30-60 mm with mixing at room temperature. In some embodiments, samples are centrifuged at 110,000 × g for 15 min at 4° C. In some embodiments, pellets are resuspended in PBS and stored at -20° C.

In certain aspects, a method of forming (e.g., preparing) isolated bacterial pmEVs, described herein, comprises the steps of: (a) centrifuging a bacterial culture, thereby forming a first pellet and a first supernatant, wherein the first pellet comprises cells; (b) discarding the first supernatant;(c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated bacterial pmEVs.

In some embodiments, the method further comprises the steps of: (11) centrifuging the solution of step (g), thereby forming a fourth pellet and a fourth supernatant; (i) discarding the fourth supernatant and resuspending the fourth pellet in a third solution. In some embodiments, the method further comprises the steps of: (j) centrifuging the solution of step (i), thereby forming a fifth pellet and a fifth supernatant; and (k) discarding the fifth supernatant and resuspending the fifth pellet in a fourth solution,

In some embodiments, the centrifugation of step (a) is at 10,000 × g. In some embodiments the centrifugation of step (a) is for 10-15 minutes. In some embodiments, the centrifugation of step (a) is at 4° C. or room temperature. In some embodiments, step (b) further comprises freezing the first pellet at -80° C. . In some embodiments, the solution in step (c) is 100mM Tris-HCl, pH 7.5 supplemented with 1 mg/ml DNa.sel, In some embodiments, the solution in step (c) is 10niM Tris-HCl, pH 8.0, 1 mM EDTA, supplemented with 0.1 mg/ml lysozyme. In some embodiments, step (c) further comprises incubating for 30 minutes at 37° C. or room temperature. In some embodiments, step (c) further comprises freezing the first pellet at -80° C. In some embodiments, step (c) further comprises adding DNase I to a final concentration of 1.6 mg/ml. In some embodiments, step (c) further comprises adding MgCb to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000 × g. In some embodiments, the centrifugation of step (e) is for 15 minutes. In some embodiments, the centrifugation of step (e) is at 4° C. or room temperature.

In some embodiments, the centrifugation of step (f) is at 120,000 × g. In some embodiments, the centrifugation of step (f) is at 110,000 × g. In some embodiments, the centrifugation of step (f) is for 1 hour. In some embodiments, the centrifugation of step (f) is for 15 minutes. In some embodiments, the centrifugation of step (f) is at 4° C. or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000 × g. In some embodiments, the centrifugation of step (h) is at 110,000 × g. In some embodiments, the centrifugation of step (h) is for 1 hour. In some embodiments, the centrifugation of step (h) is for 15 minutes. In some embodiments, the centrifugation of step (h) is at 4° C. or room temperature. In some embodiments, the third solution in step (i) is 100 mM Tris-HCl, pH 7.5. In some embodiments, the third solution in step (i) is PBS. In some embodiments, the centrifugation of step (j) is at 120,000 × g. In some embodiments, the centrifugation of step (j) is for 20 minutes. In some embodiments, the centrifugation of step (j) is at 4° C. or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000 × g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000 × g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 um filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In other embodiments, select pmEVs are isolated and/or enriched by fluorescent cell sorting by methods using affinity reagents, chemical dyes, recombinant proteins or other methods known to one skilled in the art.

The pmEVs can be analyzed, e.g., as described in Jeppesen, et al. Cell 177:428 (2019).

In some embodiments, pmEVs are lyophilized.

In some embodiments, pmEVs are gamma irradiated (e.g., at 17.5 or 25 kGy).

In some embodiments, pmEVs are UV irradiated.

In some embodiments, pmEVs are heat inactivated (e.g., at 50° C. for two hours or at 90° C. for two hours).

In some embodiments, pmEVs are acid treated.

In some embodiments, pmEVs are oxygen sparged (e.g., at 0.1 vvm for two hours).

The phase of growth can affect the amount or properties of bacteria. In the methods of pmEV preparation provided herein, pmEVs can be isolated, e.g., from a culture, at the start of the log phase of growth, midway through the log phase, and/or once stationary phase growth has been reached.

### Production of Secreted Microbial Extracellular Vesicles (smEVs)

In certain aspects, the smEVs described herein can be prepared using any method known in the art.

In some embodiments, the smEVs are prepared without an smEV purification step. For example, in some embodiments, bacteria described herein are killed using a method that leaves the smEVs intact and the resulting bacterial components, including the smEVs, are used in the methods and compositions described herein. In some embodiments, the bacteria are killed using an antibiotic (e.g., using an antibiotic described herein). In some embodiments, the bacteria are killed using UV irradiation. In some embodiments, the bacteria are heat-killed.

In some embodiments, the smEVs described herein are purified from one or more other bacterial components. Methods for purifying smEVs from bacteria are known in the art. In some embodiments, smEVs are prepared from bacterial cultures using methods described in S. Bin Park, et al. PLoS ONE. 6(3):el 7629 (2011) or G. Norheim, et al. PLoS ONE. 10(9): e0134353 (2015) or Jeppesen, et al. Cell 177:428 (2019), each of which is hereby incorporated by reference in its entirety. In some embodiments, the bacteria are cultured to high optical density and then centrifuged to pellet bacteria (e.g., at 10,000 × g for 30 min at 4° C., at 15,500 × g for 15 min at 4° C.). In some embodiments, the culture supernatants are then passed through filters to exclude intact bacterial cells (e.g., a 0.22 µm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial smEVs (e.g., at 100,000-150,000 × g for 1-3 hours at 4° C., at 200,000 × g for 1-3 hours at 4° C.). In some embodiments, the smEVs are further purified by resuspending the resulting smEV pellets (e.g., in PBS), and applying the resuspended smEVs to an Optiprep (iodixanol) gradient or gradient (e.g., a 30-60% discontinuous gradient, a 0-45% discontinuous gradient), followed by centrifugation (e.g., at 200,000 × g for 4-20 hours at 4° C.). smEV bands can be collected, diluted with PBS, and centrifuged to pellet the smEVs (e.g., at 150,000 × g for 3 hours at 4° C., at 200,000 × g for 1 hour at 4° C.). The purified smEVs can be stored, for example, at -80° C. or -20° C. until use. In some embodiments, the smEVs are further purified by treatment with DNase and/or proteinase K.

For example, in some embodiments, cultures of bacteria can be centrifuged at 11,000 × g for 20-40 min at 4° C. to pellet bacteria. Culture supernatants may be passed through a 0.22 µm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000 × g for 20-40 min at 4° C. The resulting pellets contain bacteria smEVs and other debris. Using ultracentrifugation, filtered supernatants can be centrifuged at 100,000-200,000 × g for 1-16 hours at 4° C. The pellet of this centrifugation contains bacteria smEVs and other debris such as large protein complexes. In some embodiments, using a filtration technique, such as through the use of an Amicon Ultra spin filter or by tangential flow filtration, supernatants can be filtered so as to retain species of molecular weight > 50 or 100 kDa.

Alternatively, smEVs can be obtained from bacteria cultures continuously during growth, or at selected time points during growth, for example, by connecting a bioreactor to an alternating tangential flow (ATF) system (e.g., XCell ATF, from Repligen). The ATF system retains intact cells (>0.22 um) in the bioreactor, and allows smaller components (e.g., smEVs, free proteins) to pass through a filter for collection. For example, the system may be configured so that the <0.22 um filtrate is then passed through a second filter of 100 kDa, allowing species such as smEVs between 0.22 um and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000 × g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000 × g for 3-24 hours at 4° C., e.g., 4-24 hours at 4° C.

In some embodiments, to confirm sterility and isolation of the smEV preparations, smEVs are serially diluted onto agar medium used for routine culture of the bacteria being tested, and incubated using routine conditions. Non-sterile preparations are passed through a 0.22 um filter to exclude intact cells. To further increase purity, isolated smEVs may be DNase or proteinase K treated.

In some embodiments, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 µg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of smEVs used for in vivo injections, smEVs in PBS are sterile-filtered to < 0.22 um.

In certain embodiments, to make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (200,000 × g, ≥ 3 hours, 4° C.) and resuspension.

In some embodiments, the sterility of the smEV preparations can be confirmed by plating a portion of the smEVs onto agar medium used for standard culture of the bacteria used in the generation of the smEVs and incubating using standard conditions.

In some embodiments, select sinEVs are isolated and enriched by chromatography and binding surface moieties on smEVs. In other embodiments, select smEVs are isolated and/or enriched by fluorescent cell sorting by methods using affinity reagents, chemical dyes, recombinant proteins or other methods known to one skilled in the art.

The smEVs can be analyzed, e.g., as described in Jeppesen, et al. Cell 177:428 (2019).

In some embodiments, smEVs are lyophilized.

In some embodiments, smEVs are gamma irradiated (e.g., at 17.5 or 25 kGy).

In some embodiments, smEVs are UV irradiated.

In some embodiments, smEVs are heat inactivated (e.g., at 50° C. for two hours or at 90° C. for two hours).

In some embodiments, smEVs s are acid treated.

In some embodiments, smEVs are oxygen sparged (e.g., at 0.1 vvm for two hours).

The phase of growth can affect the amount or properties of bacteria and/or smEVs produced by bacteria. For example, in the methods of smEV preparation provided herein, smEVs can be isolated, e.g., from a culture, at the start of the log phase of growth, midway through the log phase, and/or once stationary phase growth has been reached.

The growth environment (e.g., culture conditions) can affect the amount of smEVs produced by bacteria. For example, the yield of smEVs can be increased by an smEV inducer, as provided in Table 4.

TABLE 4

Culture Techniques to Increase smEV Production

| smEV inducement | smEV inducer | Acts on |
|---|---|---|
| Temperature | | |
| | Heat | stress response |
| | RT to 37° C. temp change | simulates infection |
| | 37 to 40° C. temp change | febrile infection |
| ROS | | |
| | Plumbagin | oxidative stress response |
| | Cumene hydroperoxide | oxidative stress response |
| | Hydrogen Peroxide | oxidative stress response |
| Antibiotics | | |
| | Ciprofloxacin | bacterial SOS response |
| | Gentamycin | protein synthesis |
| | Polymyxin B | outer membrane |
| | D-cylcloserine | cell will |
| Osmolyte | | |
| | NaCl | osmotic stress |
| Metal Ion Stress | | |
| | Iron Chelation | iron levels |
| | EDTA | removes divalent cations |
| | Low Hemin | iron levels |
| Media additives or removal | | |
| | Lactate | growth |
| | Ammo acid deprivation | stress |
| | Hexadecane | stress |
| | Glucose | growth |
| | Sodium bicarbonate | ToxT induction |
| | PQS | vesiculator (from bacteria) |
| | Diamines+ DFMO | membrane anchoring (negativicutes only) |
| | High nutrients | enhanced growth |
| | Low nutrients | |
| Other mechanisms | | |
| | Oxygen | oxygen stress in anaerobe |
| | No Cysteine | oxygen stress in anaerobe |
| | Inducing biofilm or floculation | |
| | Diauxic Growth | |
| | Phage | |
| | Urea | |

In the methods of smEVs preparation provided herein, the method can optionally include exposing a culture of bacteria to an smEV inducer prior to isolating smEVs from the bacterial culture. The culture of bacteria can be exposed to an smEV inducer at the start of the log phase of growth, midway through the log phase, and/or once stationary phase growth has been reached.

### Solid Dosage Form Compositions

In certain embodiments, provided herein are solid dosage forms (e.g., pharmaceutical products having a solid dosage form) comprising a pharmaceutical agent that contains bacteria and/or mEVs (such as smEVs and/or pmEVs). In some embodiments, the pharmaceutical agent can optionally contain one or more additional components, such as a cryoprotectant. The pharmaceutical agent can be lyophilized (e.g., resulting in a powder). The pharmaceutical agent can be combined with one or more excipients (e.g., pharmaceutically acceptable excipients) in the solid dosage form (e.g., solid dose form).

In certain aspects provided herein are solid dosage forms of pharmaceutical compositions. In certain embodiments, the solid dosage form comprises a pharmaceutical agent (e.g., bacteria and/or an agent (e.g., component) of bacterial origin, such as mEVs, a powder comprising bacteria and/or an agent (e.g., component) of bacterial origin, such as mEVs) and one or more disintegration agents. In certain embodiments, the total pharmaceutical agent mass is at least 0.5%, 1%, 10%, 20%, 40%, 60%, or 70% of the total mass of the pharmaceutical composition. In some embodiments the total pharmaceutical agent mass is no more than 85%, 80%, 75%, or 70% of the total mass of the pharmaceutical composition. In some embodiments, the total mass of the one or more disintegrating agents is at least 5%, at least, 6%, at least 7%, at least 8%, at least 9%, at least 10%, at least 11%, or at least 12% of the total mass of the pharmaceutical composition. In some embodiments, the total mass of the one or more disintegrating agents is no more than 12%, 11%, 10%, 9%, or 8% of the total mass of the pharmaceutical composition.

In some embodiments provided herein, the disintegrating agent is selected from natural starch, a pregelatinized starch, a sodium starch, methylcrystalline cellulose, methylcellulose, croscarmellose, croscarmellose sodium, cross-linked sodium carboxymethylcellulose, crosslinked carboxymethylcellulose, cross-linked croscarmellose, cross-linked starch such as sodium starch glycolate, low-substituted hydroxypropyl cellulose, crospovidone, polyvinylpyrrolidone, sodium alginate, a clay, or a gum. In certain preferred embodiments, the one or more disintegration agents comprise low-substituted hydroxypropyl cellulose (L-HPC, e.g., LH-11) and/or crospovidone (e.g., PVPP).

In certain embodiments, the solid dosage forms provided herein comprise L-HPC. In some embodiments, the L-HPC is of grade LH-11. In certain embodiments, the total L-HPC mass is at least 0.1%, 0.5%, 1%, 2%, 3%, 4%, 5%, 6%, 7%, 8%, 9%, or 10% of the total mass of the pharmaceutical composition. In certain embodiments, the total L-HPC mass is no more than 0.1%, 0.5%, 1%, 2%, 3%, 4%, 5%, 6%, 7%, 8%, 9%, or 10% of the total mass of the pharmaceutical composition. In certain embodiments, the total L-HPC mass is about 0.1%, 0.5%, 1%, 2%, 3%, 4%, 5%, 6%, 7%, 8%, 9%, or 10% of the total mass of the pharmaceutical composition. In certain embodiments, the total L-HPC (e.g., LH-11) mass is about 0.5% of the total mass of the pharmaceutical composition. In certain embodiments, the total L-HPC (e.g., LH-11) mass is about 5% of the total mass of the pharmaceutical composition.

In certain embodiments, the solid dosage forms provided herein comprise crospovidone (polyvinylpolypyrrolidone (PVPP), such as crospovidone CL-F). In certain embodiments, the total crospovidone (e.g., polyvinylpolypyrrolidone (PVPP), such as crospovidone CL-F)mass is at least 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., polyvinylpolypyrrolidone (PVPP), such as crospovidone CL-F)mass is no more than 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., polyvinylpolypyrrolidone (PVPP), such as crospovidone CL-F)mass is about 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., polyvinylpolypyrrolidone (PVPP), such as crospovidone CL-F)mass is about 4% to about 10% of the total mass of the pharmaceutical composition. In certain embodiments, the total crospovidone (e.g., polyvinylpolypyrrolidone (PVPP), such as crospovidone CL-F)mass is about 7% of the total mass of the pharmaceutical composition.

In certain embodiments, the solid dosage forms provided herein comprise: (i) a pharmaceutical agent having a total pharmaceutical agent mass that is at least 0.5% and no more than 75% of the total mass of the pharmaceutical composition, (ii) L-HPC (e.g., L-HPC of grade LH-11) having a total L-HPC mass that is at least 0.1% (e.g., at least 0.1%, 0.5%, 1%, 2%, 3%, 4%, 5%, 6%, 7%, 8%, 9%, or 10%) and no more than 10% (e.g., no more than 0.1%, 0. 5%, 1%, 2%, 3%, 4%, 5%, 6%, 7%, 8%, 9%, or 10%) of the total mass of the pharmaceutical composition;; and (iii) crospovidone (e.g., polyvinylpolypyrrolidone (PVPP), such as crospovidone CL-F)having a total crospovidone (e.g., polyvinylpolypyrrolidone (PVPP), such as crospovidone CL-F)mass that is at least 1% (e.g., at least 1%, 2%, 3%, 4%, 5%, 6%, 7%, 8%, 9%, 10%, 11%, 12%, 13%, 14%, or 15%) and no more than 15% (no more than 1%, 2%, 3%, 4%, 5%, 6%, 7%, 8%, 9%, 10%, 11%, 12%, 13%, 14%, or 15%) of the total mass of the pharmaceutical composition. In certain embodiments, the total L-HPC mass plus the total crospovidone (e.g., polyvinylpolypyrrolidone (PVPP), such as crospovidone CL-F)mass is at least 5%, 6%, 7%, 8%, 9%, or 10% of the total mass of the pharmaceutical composition. In some embodiments, the solid dosage form comprises: a total L-HPC mass is about 0.5% of the total mass of the pharmaceutical composition; and a total crospovidone (e.g., polyvinylpolypyrrolidone (PVPP), such as crospovidone CL-F)mass is about 7% of the total mass of the pharmaceutical composition. In some embodiments, the solid dosage form comprises: a total L-HPC mass is about 5% of the total mass of the pharmaceutical composition; and a total crospovidone (e.g., polyvinylpolypyrrolidone (PVPP), such as crospovidone CL-F)mass is about 7% of the total mass of the pharmaceutical composition.

In certain embodiments, the solid dosage forms provided herein further comprise mannitol. In some embodiments, the mannitol is mannitol SD200. In certain embodiments, the total mannitol mass is at least 25%, 30%, 35%, 40%, 45%, 50%, 55%, 60%, 65%, 70%, or 75% of the total mass of the pharmaceutical composition. In certain embodiments, the total mannitol mass is no more than 25%, 30%, 35%, 40%, 45%, 50%, 55%, 60%, 65%, 70%, 75%, 80%, 85%, 90%, or 95% of the total mass of the pharmaceutical composition. In certain embodiments, the total mannitol mass is about 25%, 30%, 35%, 40%, 45%, 50%, 55%, 60%, 65%, 70%, 75%, 80%, 85%, 90%, or 95% of the total mass of the pharmaceutical composition. In certain embodiments, the total mannitol (e.g., mannitol SD200) mass is about 26% to about 85% of the total mass of the pharmaceutical composition. In certain embodiments, the total mannitol (e.g., mannitol SD200) mass is about 26.5% of the total mass of the pharmaceutical composition. In certain embodiments, the total mannitol (e.g., mannitol SD200) mass is about 36.5% of the total mass of the pharmaceutical composition. In certain embodiments, the total mannitol (e.g., mannitol SD200) mass is about 56.5% of the total mass of the pharmaceutical composition. In certain embodiments, the total mannitol (e.g., mannitol SD200) mass is about 61% of the total mass of the pharmaceutical composition. In certain embodiments, the total mannitol (e.g., mannitol SD200) mass is about 70.5% of the total mass of the pharmaceutical composition. In certain embodiments, the total mannitol (e.g., mannitol SD200) mass is about 76% of the total mass of the pharmaceutical composition. In certain embodiments, the total mannitol (e.g., mannitol SD200) mass is about 80.5% of the total mass of the pharmaceutical composition. In certain embodiments, the total mannitol (e.g., mannitol SD200) mass is about 81.5% of the total mass of the pharmaceutical composition. In certain embodiments, the total mannitol (e.g., mannitol SD200) mass is about 83% of the total mass of the pharmaceutical composition. In certain embodiments, the total mannitol (e.g., mannitol SD200) mass is about 84.9% of the total mass of the pharmaceutical composition.

In certain embodiments, the solid dosage forms provided herein comprise magnesium stearate. In certain embodiments, the total magnesium stearate mass is at least 0.01%, 0.1%, 1%, 2%, 3%, 4%, 5%, 6%, 7%, 8%, 9%, 10%, or 11% of the total mass of the pharmaceutical composition. In certain embodiments, the total magnesium stearate mass is no more than 0.01%, 0.1%, 1%, 2%, 3%, 4%, 5%, 6%, 7%, 8%, 9%, 10%, or 11% of the total mass of the pharmaceutical composition. In certain embodiments, the total magnesium stearate mass is about 0.01%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or 11% of the total mass of the pharmaceutical composition. In certain embodiments, the total magnesium stearate mass is about 0.5% to about 1.5% of the total mass of the pharmaceutical composition. In certain embodiments, the total magnesium stearate mass is about 0.5% of the total mass of the pharmaceutical composition. In certain embodiments, the total magnesium stearate mass is about 1% of the total mass of the pharmaceutical composition. In certain embodiments, the total magnesium stearate mass is about 1.5% of the total mass of the pharmaceutical composition. In certain embodiments, the total magnesium stearate mass is about 2% of the total mass of the pharmaceutical composition.

In certain embodiments, the solid dosage forms provided herein comprise colloidal silica dioxide. In some embodiments, the colloidal silica dioxide is Aerosil 200. In certain embodiments, the total colloidal silica dioxide mass is at least 0.01%, 0.1%, 1%, 2%, 3%, 4%, 5%, 6%, 7%, 8%, 9%, 10%, or 11% of the total mass of the pharmaceutical composition. In certain embodiments, the total colloidal silica dioxide mass is no more than 0.01%, 0.1%, 1%, 2%, 3%, 4%, 5%, 6%, 7%, 8%, 9%, 10%, or 11% of the total mass of the pharmaceutical composition. In certain embodiments, the total colloidal silica dioxide mass is about 0.01%, 0.1%, 0.5%, 1%, 2%, 3%, 4%, 5%, 6%, 7%, 8%, 9%, 10%, or 11% of the total mass of the pharmaceutical composition. In certain embodiments, the total colloidal silica dioxide mass is about 0.5% to about 5% of the total mass of the pharmaceutical composition. In certain embodiments, the total colloidal silica dioxide mass is about 0.5% of the total mass of the pharmaceutical composition. In certain embodiments, the total colloidal silica dioxide mass is about 1% of the total mass of the pharmaceutical composition. In certain embodiments, the total colloidal silica dioxide mass is about 5% of the total mass of the pharmaceutical composition.

Thus, in certain embodiments, provided herein are solid dosage forms comprising a pharmaceutical agent that contains bacteria. The bacteria can be live bacteria (e.g., powder or biomass thereof); non-live (dead) bacteria (e.g., powder or biomass thereof); non replicating bacteria (e.g., powder or biomass thereof); gamma irradiated bacteria (e.g., powder or biomass thereof); and/or lyophilized bacteria (e.g., powder or biomass thereof).

In certain embodiments, the total pharmaceutical agent mass is at least 5% and no more than 25% of the total mass of the pharmaceutical composition; the total mannitol mass is at least 61% and no more than 80.5% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is at least 1.5% and no more than 2% of the total mass of the pharmaceutical composition; and the total colloidal silicon dioxide mass is about 0.5% of the total mass of the pharmaceutical composition.

In certain embodiments, the total pharmaceutical agent mass is at least 5% and no more than 60% of the total mass of the pharmaceutical composition; the total mannitol mass is at least 26.5% and no more than 81.5% of the total mass of the pharmaceutical composition; the total L-HPC mass is about 50% of the total mass of the pharmaceutical composition; the total crospovidone (e.g., polyvinylpolypyrrolidone (PVPP), such as crospovidone CL-F)mass is 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

In certain embodiments, the total pharmaceutical agent mass is at least 3% and no more than 50% of the total mass of the pharmaceutical composition; the total mannitol mass is at least 36.5% and no more than 84.9% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

In certain embodiments, the total pharmaceutical agent mass is at least 10% and no more than 50% of the total mass of the pharmaceutical composition; the total mannitol mass is at least 56.5% and no more than 76% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

In certain embodiments, the total pharmaceutical agent mass is about 50% of the total mass of the pharmaceutical composition; the total mannitol mass is about 36.5% of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1% of the total mass of the pharmaceutical composition; and the total colloidal silicon dioxide mass is about 0.5% of the total mass of the pharmaceutical composition.

In certain embodiments, the total pharmaceutical agent mass is at least 5% and no more than 60% of the total mass of the pharmaceutical composition; the total mannitol mass is at least 26% and no more than 81 % of the total mass of the pharmaceutical composition; the total L-HPC mass is about 5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1.5% of the total mass of the pharmaceutical composition; and the total colloidal silicon dioxide mass is about 0.5% of the total mass of the pharmaceutical composition.

In certain embodiments, the total pharmaceutical agent mass is about 0.5% of the total mass of the pharmaceutical composition; the total mannitol mass is about 90.5% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

In certain embodiments, the total pharmaceutical agent mass is about 5% of the total mass of the pharmaceutical composition; the total mannitol mass is about 86% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

In certain embodiments, the total pharmaceutical agent mass is about 25% of the total mass of the pharmaceutical composition; the total mannitol mass is about 66% of the total mass of the pharmaceutical composition; the total crospovidone (e.g., polyvinylpolypyrrolidone (PVPP), such as crospovidone CL-F)mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

In certain embodiments, provided herein are solid dosage forms comprising a pharmaceutical agent that contains mEVs. The mEVs can be from culture media (e.g., culture supernatant). The mEVs can be from live bacteria (e.g., powder or biomass thereof); the mEVs can be from non-live (dead) bacteria (e.g., powder or biomass thereof); the mEVs can be from non-replicating bacteria (e.g., powder or biomass thereof); the mEVs can be from gamma irradiated bacteria (e.g., powder or biomass thereof); and/or the mEVs can be from lyophilized bacteria (e.g., powder or biomass thereof).

In some embodiments, the pharmaceutical agent comprises mEVs substantially or entirely free of bacteria (e.g., whole bacteria) (e.g., live bacteria, dead (e.g., killed) bacteria, non-replicating bacteria, attenuated bacteria. In some embodiments, the pharmaceutical compositions comprise both mEVs and bacteria (e.g., whole bacteria) (e.g., live bacteria, killed bacteria, attenuated bacteria). In some embodiments, the pharmaceutical agents comprise bacteria and/or mEVs from one or more (e.g., 1, 2, 3, 4, 5, 6, 7, 8, 9, 10, or more) of the bacteria strains or species or taxonomic groups listed herein. In some embodiments, the pharmaceutical agents comprise bacteria and/or mEVs from one of the bacteria strains or species or taxonomic groups listed herein. In some embodiments, the pharmaceutical agents comprise lyophilized bacteria and/or mEVs. In some embodiments, the pharmaceutical agent comprises gamma irradiated bacteria and/or mEVs. The mEVs (such as smEVs and/or pmEVs) can be gamma irradiated after the mEVs are isolated (e.g., prepared). In some embodiments, the pharmaceutical agents comprise bacteria and/or mEVs from one of the bacteria strains or species described herein, e.g., Lactococcus, Prevotella, Bifidobacterium, Veillonella, Fournierella, Harryflintia, Megasphaera; e.g., Lactococcus lactis cremoris; Prevotella histicola; Bifidobacterium animalis lactis; Veillonella parvula; Fournierella massiliensis; Harryflintia acetispora; or Megasphaera sp.

In some embodiments, to quantify the numbers of mEVs (such as smEVs and/or pmEVs) and/or bacteria present in a sample, electron microscopy (e.g., EM of ultrathin frozen sections) can be used to visualize the mEVs (such as smEVs and/or pmEVs) and/or bacteria and count their relative numbers. Alternatively, nanoparticle tracking analysis (NTA), Coulter counting, or dynamic light scattering (DLS) or a combination of these techniques can be used. NTA and the Coulter counter count particles and show their sizes. DLS gives the size distribution of particles, but not the concentration. Bacteria frequently have diameters of 1-2 um (microns). The full range is 0.2-20 um. Combined results from Coulter counting and NTA can reveal the numbers of bacteria and/or mEVs (such as smEVs and/or pmEVs) in a given sample. Coulter counting reveals the numbers of particles with diameters of 0.7-10 um. For most bacterial and/or mEV (such as smEV and/or pmEV) samples, the Coulter counter alone can reveal the number of bacteria and/or mEVs (such as smEVs and/or pmEVs) in a sample. pmEVs are 20-600 nm in diameter. For NTA, a Nanosight instrument can be obtained from Malvern Pananlytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm - 3 um.

mEVs can be characterized by analytical methods known in the art (e.g., Jeppesen, et al. Cell 177:428 (2019)).

In some embodiments, the bacteria and/or mEVs may be quantified based on particle count. For example, total particle count of a bacteria and/or mEV preparation can be measured using NTA.

In some embodiments, the bacteria and/or mEVs may be quantified based on the amount of protein, lipid, or carbohydrate. For example, total protein content of a bacteria and/or preparation can be measured using the Bradford assay or BCA.

In some embodiments, mEVs are isolated away from one or more other bacterial components of the source bacteria or bacterial culture. In some embodiments, bacteria are isolated away from one or more other bacterial components of the source bacterial culture. In some embodiments, the pharmaceutical agent further comprises other bacterial components.

In certain embodiments, the mEV preparation obtained from the source bacteria may be fractionated into subpopulations based on the physical properties (e.g., sized, density, protein content, binding affinity) of the subpopulations. One or more of the mEV subpopulations can then be incorporated into the pharmaceutical agents of the invention.

In certain aspects, provided herein are solid dosage forms comprising pharmaceutical agents that comprise bacteria and/or mEVs (such as smEVs and/or pmEVs) useful for the treatment and/or prevention of disease (e.g., a cancer, an autoimmune disease, an inflammatory disease, a metabolic disease, or a dysbiosis), as well as methods of making and/or identifying such bacteria and/or mEVs, and methods of using pharmaceutical agents and solid dosage forms thereof (e.g., for the treatment of a cancer, an autoimmune disease, an inflammatory disease, or a metabolic disease, either alone or in combination with other therapeutics). In some embodiments, the pharmaceutical agents comprise both mEVs (such as smEVs and/or pmEVs) and bacteria (e.g., whole bacteria) (e.g., live bacteria, dead (e.g., killed) bacteria, non-replicating bacteria, attenuated bacteria). In some embodiments, the pharmaceutical agents comprise bacteria in the absence of mEVs (such as smEVs and/or pmEVs). In some embodiments, the pharmaceutical agents comprise mEVs (such as smEVs and/or pmEVs) in the absence of bacteria. In some embodiments, the pharmaceutical agents comprise mEVs (such as smEVs and/or pmEVs) and/or bacteria from one or more of the bacteria strains or species or taxonomic groups listed herein. In some embodiments, the pharmaceutical compositions comprise mEVs (such as smEVs and/or pmEVs) and/or bacteria from one of the bacteria strains or species or taxonomic groups listed herein. In some embodiments, the pharmaceutical agents comprise bacteria and/or mEVs from one of the bacteria strains or species described herein, e.g., Lactococcus, Prevotella, Bifidobacterium, Veillonella, Fournierella, Harryflintia, Megasphaera; e.g., Lactococcus lactis cremoris; Prevotella histicola; Bifidobacterium animalis lactis; Veillonella parvula; Fournierella massiliensis; Harryflintia acetispora; or Megasphaera sp.

In certain aspects, provided are pharmaceutical agents for administration to a subject (e.g., human subject). In some embodiments, the pharmaceutical agents are combined with additional active and/or inactive materials in order to produce a final product, which may be in single dosage unit or in a multi-dose format. In some embodiments, the pharmaceutical agent is combined with an adjuvant such as an immuno-adjuvant (e.g., a STING agonist, a TLR agonist, or a NOD agonist).

In some embodiments, the solid dosage form comprises at least one carbohydrate.

In some embodiments, the solid dosage form comprises at least one lipid. In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20: 1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the solid dosage form comprises at least one mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the solid dosage form comprises at least one vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the solid dosage form comprises an excipient. Nonlimiting examples of suitable excipients include a buffering agent, a preservative, a stabilizer, a binder, a compaction agent, a lubricant, a dispersion enhancer, a disintegration agent, a flavoring agent, a sweetener, and a coloring agent.

Suitable excipients that can be included in the solid dosage form can be one or more pharmaceutically acceptable excipients known in the art. For example, see Rowe, Sheskey, and Quinn, eds., Handbook of Pharmaceutical Excipients, sixth ed.; 2009; Pharmaceutical Press and American Pharmacists Association.

### Solid Dosage Forms

The solid dosage form described herein can be, e.g., a tablet or a minitablet. Further, a plurality of minitablets can be in (e.g., loaded into) a capsule.

In some embodiments, the solid dosage form comprises a tablet (> 4 mm) (e.g., 5 mm-17 mm). For example, the tablet is a 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm,17 mm or 18 mm tablet. The size refers to the diameter of the tablet, as is known in the art. As used herein, the size of the tablet refers to the size of the tablet prior to application of an enteric coating.

In some embodiments, the solid dosage form comprises a minitablet. The minitablet can be in the size range of 1 mm-4 mm range. E.g., the minitablet can be a 1 mm minitablet, 1.5 mm minitablet, 2 mm minitablet, 3 mm minitablet, or 4 mm minitablet. The size refers to the diameter of the minitablet, as is known in the art. As used herein, the size of the minitablet refers to the size of the minitablet prior to application of an enteric coating.

The minitablets can be in a capsule. The capsule can be a size 00, size 0, size 1, size 2, size 3, size 4, or size 5 capsule. The capsule that contains the minitablets can comprise HPMC (hydroxyl propyl methyl cellulose) or gelatin. The minitablets can be inside a capsule: the number of minitablets inside a capsule will depend on the size of the capsule and the size of the minitablets. As an example, a size 0 capsule can contain 31-35 (an average of 33) minitablets that are 3 mm minitablets. In some embodiments, the capsule is banded after loading. In some embodiments, the capsule is banded with an HPMC-based banding solution.

### Coating

The solid dosage form (e.g., tablet or minitablet) described herein can be enterically coated, e.g., with one enteric coating layer or with two layers of enteric coating, e.g., an inner enteric coating and an outer enteric coating. The inner enteric coating and outer enteric coating are not identical (e.g., the inner enteric coating and outer enteric coating do not contain the same components in the same amounts). The enteric coating allows for release of the pharmaceutical agent, e.g., in the small intestine, e.g., upper small intestine, e.g., duodenum and/or jejunum.

Release of the pharmaceutical agent in the small intestine, e.g., in the upper small intestine, e.g., in the duodenum, or in the jejunum, allows the pharmaceutical agent to target and affect cells (e.g., epithelial cells and/or immune cells) located at these specific locations, e.g., which can cause a local effect in the small intestine and/or cause a systemic effect (e.g., an effect outside of the gastrointestinal tract).

EUDRAGIT is the brand name for a diverse range of polymethacrylate-based copolymers. It includes anionic, cationic, and neutral copolymers based on methacrylic acid and methacrylic/acrylic esters or their derivatives,

Examples of other materials that can be used in the enteric coating (e.g., the one enteric coating or the inner enteric coating and/or the outer enteric coating) include cellulose acetate phthalate (CAP), cellulose acetate trimellitate (CAT), poly(vinyl acetate phthalate) (PVAP), hydroxypropyl methylcellulose phthalate (HPMCP), fatty acids, waxes, shellac (esters of aleurtic acid), plastics, plant fibers, zein, AQUA-ZEIN® (an aqueous zein formulation containing no alcohol), amylose starch, starch derivatives, dextrins, methyl acrylate-methacrylic acid copolymers, cellulose acetate succinate, hydroxypropyl methyl cellulose acetate succinate (hypromellose acetate succinate), methyl methacrylate-methacrylic acid copolymers, and/or sodium alginate.

The enteric coating (e.g., the one enteric coating or the inner enteric coating and/or the outer enteric coating) can include a methacrylic acid ethyl acrylate (MAE) copolymer (1:1).

The one enteric coating can include methacrylic acid ethyl acrylate (MAE) copolymer (1:1) (such as Kollicoat MAE 100P).

The one enteric coating can include a Eudragit coplymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

Other examples of materials that can be used in the enteric coating (e.g., the one enteric coating or the inner enteric coating and/or the outer enteric coating) include those described in, e.g., U.S. 6312728; U.S. 6623759; U.S. 4775536; U.S. 5047258, U.S. 5292522; U.S. 6555124; U.S. 6638534; U.S. 2006/0210631; U.S. 2008/200482; U.S. 2005/0271778; U.S. 2004/0028737; WO 2005/044240, hereby incorporated by reference in their entirety, particularly with respect to the enteric coatings disclosed therein.

See also, e.g., U.S. 9233074, which provides pH dependent, enteric polymers that can be used with the solid dosage forms provided herein, including methacrylic acid copolymers, polyvinylacetate phthalate, hydroxypropylmethyl cellulose acetate succinate, hydroxypropylmethyl cellulose phthalate and cellulose acetate phthalate; suitable methacrylic acid copolymers include: poly(methacrylic acid, methyl methacrylate) 1:1 sold, for example, under the Eudragit L100 trade name; poly(methacrylic acid, ethyl acrylate) 1:1 sold, for example, under the Eudragit L100-55 trade name; partially-neutralized poly(methacrylic acid, ethyl acrylate) 1:1 sold, for example, under the Kollicoat MAE-100P trade name; and poly(methacrylic acid, methyl methacrylate) 1:2 sold, for example, under the Eudragit S100 trade name.

In certain aspects, the solid dosage form (e.g., tablet or minitablet) described herein further comprises a sub-coating. In some embodiments, the solid dosage form comprises a sub-coating, e.g., in addition to the enteric coating, e.g., the sub-coating is beneath the enteric coating (e.g., between the solid dosage form and the enteric coating). In some embodiments, the sub-coating comprises Opadry QX, e.g., Opadry QX Blue. The sub-coat can be used, e.g., to visually mask the appearance of the therapeutic agent.

### Dose

The dose of the pharmaceutical agent (e.g., for human subjects) is the dose per capsule or tablet or per total number of minitablets used in a capsule.

In embodiments where dose is determined by total cell count, total cell count can be determined by Coulter counter.

In some embodiments, the pharmaceutical agent comprises bacteria and the dose of bacteria is about 1 × 10⁷ to about 2 × 10¹² (e.g., about 3 × 10¹⁰ or about 1.5 × 10¹¹ or about 1.5 × 10¹²) cells (e.g., wherein cell number is determined by total cell count, which is determined by Coulter counter), wherein the dose is per capsule or tablet or per total number of minitablets in a capsule. In some embodiments, the pharmaceutical agent comprises bacteria and the dose of bacteria is about 1 × 10¹⁰ to about 2 × 10¹² (e.g., about 1.6 × 10¹¹ or about 8 × 10¹¹ or about 9.6 × 10¹¹ about 12.8 × 10H or about 1.6 × 10¹²) cells (e.g., wherein cell number is determined by total cell count, which is determined by Coulter counter), wherein the dose is per capsule or tablet or per total number of minitablets in a capsule.

In some embodiments, the pharmaceutical agent comprises bacteria and the dose of bacteria is about 1 × 10⁹, about 3 × 10⁹, about 5 × 10⁹, about 1.5 × 10¹⁰, about 3 × 10¹⁰, about 5 × 10¹⁰, about 1.5 × 10¹¹, about 1.5 × 10¹², or about 2 × 10¹² cells, wherein the dose is per capsule or tablet or per total number of minitablets in a capsule.

In some embodiments, the pharmaceutical agent comprises mEVs and the dose of mEVs is about 1 × 10⁵ to about 7 × 10¹³ particles (e.g., wherein particle count is determined by NTA (nanoparticle tracking analysis)), wherein the dose is per capsule or tablet or per total number of minitablets in a capsule. In some embodiments, the pharmaceutical agent comprises mEVs and the dose of mEVs is about 1 × 10¹⁰ to about 7 × 10¹³ particles (e.g., wherein particle count is determined by NTA (nanoparticle tracking analysis)), wherein the dose is per capsule or tablet or per total number of minitablets in a capsule.

In some embodiments, wherein the pharmaceutical agent comprises mEVs, the dose of mEVs is about 2×10⁶ to about 2×10¹⁶ particles (e.g., wherein particle count is determined by NTA (nanoparticle tracking analysis)), wherein the dose is per capsule or tablet or per total number of minitablets in a capsule.

In some embodiments, the pharmaceutical agent dose can be a milligram (mg) dose determined by weight the pharmaceutical agent (e.g., a powder comprising bacteria and/or an agent of bacterial origin, such as mEVs). The dose of the pharmaceutical agent is per capsule or tablet or per total number of minitablets, e.g., in a capsule.

For example, to administer a 1× dose of the pharmaceutical agent of about 400 mg, about 200 mg of the pharmaceutical agent is present per capsule and two capsules are administered, resulting in a dose of about 400 mg. The two capsules can be administered, for example, 1× or 2× daily.

For example, for a minitablet: about 0.1 to about 3.5 mg (0.1, 0.35, 1.0, 3.5 mg) of the pharmaceutical agent can be contained per minitablet. The minitablets can be inside a capsule: the number of minitablets inside a capsule will depend on the size of the capsule and the size of the minitablets. For example, an average of 33 (range of 31-35) 3 mm minitablets fit inside a size 0 capsule. As an example, 0.1- 3.5 mg of the pharmaceutical agent per minitablet, the dose range will be 3.3 mg- 115.5 mg (for 33 minitablets in size 0 capsule) per capsule (3.1 mg- 108.5 mg for 31 minitablets in size 0 capsule) (3.5 mg- 122.5 mg for 35 minitablets in size 0 capsule). Multiple capsules and/or larger capsule(s) can be administered to increase the administered dose and/or can be administered one or more times per day to increase the administered dose.

In some embodiments, the dose can be about 3 mg to about 125 mg of the pharmaceutical agent, per capsule or tablet or per total number of minitablets, e.g., in a capsule.

In some embodiments, the dose can be about 35 mg to about 1200 mg (e.g., about 35 mg, about 125 mg, about 350 mg, or about 1200 mg) of the pharmaceutical agent.

In some embodiments, the dose of the pharmaceutical agent can be about 30 mg to about 3500 mg (about 25, about 50, about 75, about 100, about 150, about 250, about 300, about 350, about 400, about 500, about 600, about 750, about 1000, about 1250, about 1300, about 2000, about 2500, about 3000, or about 3500 mg).

A human dose can be calculated appropriately based on allometric scaling of a dose administered to a model organism (e.g., mouse).

In some embodiments, one or two tablets capsules can be administered one or two times a day.

The pharmaceutical agent contains the bacteria and/or an agent of bacterial origin, such as mEVs, or contains a powder comprising bacteria and/or an agent of bacterial origin, such as mEVs, and can also contain one or more additional components, such as a cryoprotectantetc.

In some embodiments, the mg (by weight) dose of the pharmaceutical agent is, e.g., about 1 mg to about 500 mg per capsule, or per tablet, or per total number of minitablets, e.g., used in a capsule.

### Methods of Use

The solid dosage forms described herein allow, e.g., for oral administration of a pharmaceutical agent contained therein.

The solid dosage forms having the disclosed combinations and/or amounts of disintegration agents provide a decrease in disintegration times (e.g., 2-fold, 4-fold, 6-fold, 8-fold), which can further result in an increase in therapeutic efficacy and/or physiological effect as compared to the same solid dosage forms that do not have the disclosed combinations of the disintegration agents.

The solid dosage forms described herein can be used in the treatment and/or prevention of a cancer, inflammation, autoimmunity, a metabolic condition, or a dysbiosis.

Methods of using a solid dosage form (e.g., for oral administration) (e.g., for pharmaceutical use) comprising a pharmaceutical agent (e.g., a therapeutically effective amount thereof), wherein the pharmaceutical agent comprises bacteria and/or microbial extracellular vesicles (mEVs), and wherein the solid dosage form further comprises the disclosed disintegration agents are described herein.

The methods and administered solid dosage forms described herein allow, e.g., for oral administration of a pharmaceutical agent contained therein. The solid dosage form can be administered to a subject is a fed or fasting state. The solid dosage form can be administered, e.g., on an empty stomach (e.g., one hour before eating or two hours after eating). The solid dosage form can be administered one hour before eating. The solid dosage form can be administered two hours after eating.

A solid dosage form for use in the treatment and/or prevention of a cancer, inflammation, autoimmunity, a metabolic condition, or a dysbiosis is provided herein.

Use of a solid dosage form for the preparation of a medicament for the treatment and/or prevention of a cancer, inflammation, autoimmunity, a metabolic condition, or a dysbiosis is provided herein.

### Method of Making Solid Dosage Forms

In certain aspects, provided herein are methods of preparing a solid dosage form of a pharmaceutical composition, the method comprising combining into a pharmaceutical composition a pharmaceutical agent (e.g., bacteria disclosed herein and/or an agent (e.g., component) or a powder comprising bacteria disclosed herein and/or an agent (e.g., component)) of bacterial origin, such as mEVs disclosed herein) and one or more (e.g., one, two, or three) disintegration agents. In some embodiments, the total mass of the one or more disintegrating agents is at least 5%, at least, 6%, at least 7%, at least 8%, at least 9%, at least 10%, at least 11%, or at least 12%. In some embodiments, the total mass of the one or more disintegrating agents is no more than 12%, 11%, 10%, 9%, or 8% of the total mass of the pharmaceutical composition. In some embodiments, the one or more disintegration agents comprise low-substituted hydroxypropyl cellulose (L-HPC, e.g., LH-11) and/or crospovidone (e.g., PVPP).

In certain embodiments, the solid dosage forms provided herein comprise L-HPC. In some embodiments, the L-HPC is of grade LH-11. In certain embodiments, the total L-HPC mass is at least 0.1%, 0.5%, 1%, 2%, 3%, 4%, 5%, 6%, 7%, 8%, 9%, or 10% of the total mass of the pharmaceutical composition. In certain embodiments, the total L-HPC mass is no more than 0.1%, 0.5%, 1%, 2%, 3%, 4%, 5%, 6%, 7%, 8%, 9%, or 10% of the total mass of the pharmaceutical composition. In certain embodiments, the total L-HPC mass is about 0.1%, 0.5%, 1%, 2%, 3%, 4%, 5%, 6%, 7%, 8%, 9%, or 10% of the total mass of the pharmaceutical composition. In certain embodiments, the total L-HPC (e.g., LH- 11) mass is about 0.5%of the total mass of the pharmaceutical composition. In certain embodiments, the total L-HPC (e.g., LH- 11) mass is about 5%of the total mass of the pharmaceutical composition.

In certain embodiments, the solid dosage forms provided herein comprise crospovidone (polyvinylpolypyrrolidone (PVPP), such as crospovidone CL-F). In certain embodiments, the total crospovidone (e.g., polyvinylpolypyrrolidone (PVPP), such as crospovidone CL-F)mass is at least 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., polyvinylpolypyrrolidone (PVPP), such as crospovidone CL-F)mass is no more than 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., polyvinylpolypyrrolidone (PVPP), such as crospovidone CL-F)mass is about 1%, 2%, 3%, 4%, 5%, 6%, 7%, 8%, 9%, 10%, 11%, 12%, 13%, 14%, or 15% of the total mass of the pharmaceutical composition. In certain embodiments, the total crospovidone (e.g., polyvinylpolypyrrolidone (PVPP), such as crospovidone CL-F)mass is about 4% to about 10% of the total mass of the pharmaceutical composition. In certain embodiments, the total crospovidone (e.g., polyvinylpolypyrrolidone (PVPP), such as crospovidone CL-F) mass is about 7% of the total mass of the pharmaceutical composition.

In certain embodiments, the method further comprises compressing the pharmaceutical composition, thereby forming a tablet or a minitablet. In some embodiments, the method further comprises enterically coating the tablet or minitablet, thereby preparing the enterically coated tablet. In certain embodiments, the method further comprises loading the minitablets into a capsule.

### Additional Aspects of the Solid Dosage Forms

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described disintegration agents, can provide a therapeutically effective amount of the pharmaceutical agent to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described disintegration agents, can provide a non-natural amount of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described disintegration agents, can provide an unnatural quantity of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described disintegration agents, can bring about one or more changes to a subject, e.g., human, e.g., to treat or prevent a disease or a health disorder.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described disintegration agents, has potential for significant utility, e.g., to affect a subject, e.g., a human, e.g., to treat or prevent a disease or a health disorder.

### Additional Therapeutic Agents

In certain aspects, the methods provided herein include the administration to a subject of a solid dosage form described herein either alone or in combination with an additional therapeutic agent. In some embodiments, the additional therapeutic agent is an immunosuppressant, an anti-inflammatory agent, a steroid, and/or a cancer therapeutic.

In some embodiments, the solid dosage form is administered to the subject before the additional therapeutic agent is administered (e.g., at least 1, 2, 3, 4, 5, 6, 7, 8, 9, 10, 11, 12, 13, 14, 15, 16, 17, 1 8, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments , the solid dosage form is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the solid dosage form and the additional therapeutic agent are administered to the subject simultaneously or nearly simultaneously (e.g., administrations occur within an hour of each other).

In some embodiments, an antibiotic is administered to the subject before the solid dosage form is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after the solid dosage form is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the solid dosage form and the antibiotic are administered to the subject simultaneously or nearly simultaneously (e.g., administrations occur within an hour of each other).

In some embodiments, the additional therapeutic agent is a cancer therapeutic. In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesm synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall ; dynemicin, including dynemicm A; bisphosphonates, such as clodronate; an esperamicin; as well as neocarzinostatin chromophore and related chromoprotein enedlytie antibiotic chromophores, aclacinomysins, actinomycin, authrarnycin, azaserine, bleomycins, cactinomycin, carabicin, caminomycin, carzinophilin, chromomycmis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicm, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabme, 6-mercaptopurine, thiamiprine, thioguanine; pyrimidine analogs such as ancitabine, azacitidine, 6-azauridine, carmofur, cytarabine, dideoxyuridme, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannoniustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the cancer therapeutic is a cancer immunotherapy agent. Immunotherapy refers to a treatment that uses a subject’s immune system to treat cancer, e.g., checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), lpiliniuniab (BMS, anti-CTLA-4), MED14736 (AstraZeneca, anti-PD-Ll), and MPDL3280A (Roche, anti-PD-LI). Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. The immunotherapy agent may be administered via injection (e.g., intravenously, intratumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STT-A1010.

In some embodiments, the methods provided herein include the administration of a pharmaceutical composition described herein in combination with one or more additional therapeutic agents. In some embodiments, the methods disclosed herein include the administration of two immunotherapy agents (e.g., immune checkpoint inhibitor). For example, the methods provided herein include the administration of a pharmaceutical composition described herein in combination with a PD-1 inhibitor (such as pemrolizumab or nivolumab or pidilizumab) or a CLTA-4 inhibitor (such as ipilimumab) or a PD-L1 inhibitor.

In some embodiments, the immunotherapy agent is an antibody or antigen binding fragment thereof that, for example, binds to a cancer-associated antigen. Examples of cancer-associated antigens include, but are not limited to, adipophilin, AIM-2, ALDH1Al, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-Al, dek-can fusion protein, DKK1, EFTUD2, Elongation factor 2, ENAH (hMena), Ep-CAM, EpCAM, EphA3, epithelial tumor antigen (“ETA”), E′I′V6-Al\/ILI fusion protein, EZH2, FGF5, FLT3-ITD, FN1, G250/MN/CAIX, GAGE-1,2,8, GAGE-3,4,5,6,7, GAS7, glypicati-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHNI also known as CCDC110, LAGE-1, LDLR-fucosyltransferaseAS fusion protein, Lengsin, M-CSF, MAGE-Al, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-⅟LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-l/gp75, TRP-2, TRP2- INT2, tyrosinase, tyrosinase (“TYR”), VEGF, WT1, XAGE-lb/GAGED2a. In some embodiments, the antigen is a neo-antigen.

In some embodiments, the immunotherapy agent is a cancer vaccine and/or a component of a cancer vaccine (e.g., an antigenic peptide and/or protein). The cancer vaccine can be a protein vaccine, a nucleic acid vaccine or a combination thereof. For example, in some embodiments, the cancer vaccine comprises a polypeptide comprising an epitope of a cancer-associated antigen. In some embodiments, the cancer vaccine comprises a nucleic acid (e.g., DNA or RNA, such as mRNA) that encodes an epitope of a cancer-associated antigen. Examples of cancer-associated antigens include, but are not limited to, adipophilin, AIM-2, ALDHIAl, alpha-actinin-4, alpha-fetoprotein (“AFP”), ARTCI, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIAl, CTAG1, CTAG2, cyclin DI, Cyclin-Al, dek-can fusion protein, DKK1, EFTUD2, Elongation factor 2, ENAH (hMena), Ep-CAM, EpCAM, EpliA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, 1-H-A-Al 1, HLA-A2, HLA-DOB, hsp70-2, IDOl, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, TvLkGE-Cl., MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-⅟LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-⅟gp75, TRP-2, TRP2-INT2, tyrosinase, tyrosinase (“TYR”), VEGF, WT1, XAGE-1b/GAGED2a. In some embodiments, the antigen is a neo-antigen. In some embodiments, the cancer vaccine is administered with an adjuvant. Examples of adjuvants include, but are not limited to, an immune modulatory protein, Adjuvant 65, α-GalCer, aluminum phosphate, aluminum hydroxide, calcium phosphate, β-Glucan Peptide, CpG ODN DNA, GPI-0100, lipid A, lipopolysaccharide, Lipovant, Montanide, N-acetyl-muramyl-L-alanyl-D-isoglutamine, Pam3CSK4, quil A , cholera toxin (CT) and heat-labile toxin from enterotoxigenic Escherichia coli (LT) including derivatives of these (CTB, mmCT, CTA1-DD, LTB, LTK63, LTR72, dmLT) and trehalose dimycolate.

In some embodiments, the immunotherapy agent is an immune modulating protein to the subject. In some embodiments, the immune modulatory protein is a cytokine or chemokine. Examples of immune modulating proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon alpha (“IFN-alpha”), Interferon beta (“IFN-beta”) Interferon gamma (“IFN-gamma”), Interlukin-1 alpha (“IL-1 alpha”), Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin- 10 (“IL-10”), Interleukin- 11 (“IL-11”), Subunit beta of Interleukin- 12 (“IL-12 p40” or “IL-12 p70”), Interleukin-13 (“IL-13”), Interleukin-15 (“IL-15”), Interleukin-16 (“IL-16”), Interleukin-17A-F (“IL-17A-F”), Interleukin-18 (“IL-18”), Interleukin-21 (“IL-21”), Interleukin-22 (“IL-22”), Interleukin-23 (“IL-23”), Interleukin-33 (“IL-33”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein- 1 -delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4“), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin- 18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor- 1 alpha (“SDF-1 alpha”), Chemokine (C-C motif) ligand 17 (“TARC”), Thymus-expressed chemokine (“TECK”), Thymic stromal lymphopoietin (“TSLP 4- IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 Rl, IL-13R2, IL-17B, IL-2 Ra, IL-2 Rb, IL-23, LAP, Neuronal cell adhesion molecule (“NrCAM”), Plasminogen activator inhibitor- 1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFRIAdiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/ Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD 144 (“VE-Cadherin”), WNT1-inducible-signaling pathway protein 1 (“WISP-1”), and Receptor Activator of Nuclear Factor κ B (“RANK”).

In some embodiments, the cancer therapeutic is an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 1311-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

Exemplary anti-cancer compounds that modify the function of proteins that regulate gene expression and other cellular functions (e.g., HDAC inhibitors, retinoid receptor ligants) are Vorinostat (Zolinza®), Bexarotene (Targretin®) and Romidepsin (Istodax®), Alitretinoin (Panretin®), and Tretinoin (Vesanoid®).

Exemplary anti-cancer compounds that induce apoptosis (e.g., proteasome inhibitors, antifolates) are Bortezomib (Velcade®), Carfilzomib (Kyprolis™), and Pralatrexate (Folotyn®).

Exemplary anti-cancer compounds that increase anti-tumor immune response (e.g., anti CD20, anti CD52; anti-cytotoxic T-lymphocyte-associated antigen-4) are Rituximab (Rituxan®), Alemtuzumab (Campath®), Ofatumumab (Arzerra®), and Ipilimumab (Yervoy™).

Exemplary anti-cancer compounds that deliver toxic agents to cancer cells (e.g., anti-CD20-radionuclide fusions; IL-2-diphtheria toxin fusions; anti-CD30- monomethylauristatin E (MMAE)-fusions) are Tositumomab and 131I-tosittimoniab (Bexxar®) and Ibritumomab tiuxetan (Zevalin®), Denileukin diftitox (Ontak®), and Brentuximab vedotin (Adcetris®).

Other exemplary anti-cancer compounds are small molecule inhibitors and conjugates thereof of, e.g., Janus kinase, ALK, Bcl-2, PARP, PI3K, VEGF receptor, Braf, MEK, CDK, and HSP90.

Exemplary platinum-based anti-cancer compounds include, for example, cisplatin, carboplatin, oxaliplatin, satraplatin, picoplatin, Nedaplatin, Triplatin, and Lipoplatin. Other metal-based drugs suitable for treatment include, but are not limited to ruthenium-based compounds, ferrocene derivatives, titanium-based compounds, and gallium-based compounds.

In some embodiments, the cancer therapeutic is a radioactive moiety that comprises a radionuclide. Exemplary radionuclides include, but are not limited to Cr-51, Cs-131, Ce-134, Se-75, Ru-97, 1-125, Eu-149, Os-189m, Sb-119, I-123, Ho-161, Sb-117, Ce-139, In-111, Rh-103m, Ga-67, Tl-201, Pd-103, Au-195, Hg-197, Sr-87m, Pt-191, P-33, Er-169, Ru-103, Yb-169, Au-199, Sn-121, Tm-167, Yb-175, In-113m, Sn-113, Lu-177, Rh-105, Sn-117m, Cu-67, Sc-47, Pt-195m, Ce-141, I-131, Tb-161, As-77, Pt-197, Sm-153, Gd-159, Tm-173, Pr-143, Au-198, Tm-170, Re-186, Ag-111, Pd-109, Ga-73, Dy-165, Pm-149, Sn-123, Sr-89, Ho-166, P-32, Re-188, Pr-142, Ir-194, In-114m/In-114, and Y-90.

In some embodiments, the additional therapeutic is an antibiotic. For example, if the presence of a disease-associated bacteria and/or a disease-associated microbiome profile is detected, antibiotics can be administered, e.g., to eliminate the disease-associated bacteria from the subject. In some embodiments, the cancer therapeutic is an antibiotic. For example, if the presence of a cancer-associated bacteria and/or a cancer-associated microbiome profile is detected according to the methods provided herein, antibiotics can be administered to eliminate the cancer-associated bacteria from the subject. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a disease (such as cancer) niche may be used to target disease-associated microbes, including disease-associated bacteria in that niche. In other embodiments, antibiotics are administered after the solid dosage form. In some embodiments, antibiotics are administered before the solid dosage form.

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin, Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillim/clavulanate, ampicillin/sulbactam, amphomycin ristocetin, azithromycin, bacitracin, buforin II, carbomycin, cecropin P1, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic agent is an immunosuppressive agent, a DMARD, a pain-control drug, a steroid, a non-steroidal antiinflammatory drug (NSAID),, or a cytokine antagonist, and combinations thereof. Representative agents include, but are not limited to, cyclosporin, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra /Actemra®), integrin antagonists (TYSABRI® (natalizumab)), IL-1 antagonists (ACZ885 (Ilaris)), Anakinra (Kineret®)), CD4 antagonists, IL-23 antagonists, IL-20 antagonists, IL-6 antagonists, BLyS antagonists (e.g., Atacicept, Benlysta®/ LymphoStat-B® (belimumab)), p38 Inhibitors, CD20 antagonists (Ocrelizumab, Ofatumumab (Arzerra®)), interferon gamma antagonists (Fontolizumab), prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin + Viusid, TwHF, Methoxsalen, Vitamin D - ergocalciferol, Milnacipran, Paclitaxel, rosig tazone, Tacrolimus (Prograf®), RADOO1, rapamune, rapamycin, fostamatinib, Fentanyl, XOMA 052, Fostamatinib disodium, rosightazone, Curcumin (Longvida™), Rosuvastatin, Maraviroc, ramipnl, Milnacipran, Cobiprostone, somatropin, tgAAC94 gene therapy vector, MK0359, GW856553, esomeprazole, everolimus, trastuzumab, JAK1 and JAK2 inhibitors, pan JAK inhibitors, e.g., tetracyclic pyridone 6 (P6), 325, PF-956980, denosumab, IL-6 antagonists, CD20 antagonists, CTLA4 antagonists, IL-8 antagonists, IL-21 antagonists, IL-22 antagonist, integrin antagonists (Tysarbri® (natalizumab)), VGEF antagnosits, CXCL antagonists, MMP antagonists, defensin antagonists, IL-1 antagonists (including IL-1 beta antagonsits), and IL-23 antagonists (e.g., receptor decoys, antagonistic antibodies, etc.).

In some embodiments, the additional therapeutic agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

In some embodiments, the additional therapeutic agent is an RNAmolecule, such as a double stranded RNA.

In some embodiments, the additional therapeutic agent is an anti-sense oligonucleotide.

### Administration

In certain aspects, provided herein is a method of delivering a solid dosage form described herein to a subject. In some embodiments of the methods provided herein, the solid dosage form that comprises bacteria and/or mEVs is administered in conjunction with the administration of an additional therapeutic agent. In some embodiments, the solid dosage form comprises a pharmaceutical agent co-formulated with the additional therapeutic agent. In some embodiments, the solid dosage form is co-administered with the additional therapeutic agent. In some embodiments, the additional therapeutic agent is administered to the subject before administration of the solid dosage form (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the solid dosage form (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the solid dosage form and the additional therapeutic agent. In some embodiments, different modes of delivery are used to administer the solid dosage form and the additional therapeutic agent. For example, in some embodiments the solid dosage form is administered orally while the additional therapeutic agent is administered via injection (e.g., an intravenous, intramuscular and/or intratumoral injection).

In certain embodiments, the solid dosage form described herein can be administered in conjunction with any other conventional anti-cancer treatment, such as, for example, radiation therapy and surgical resection of the tumor. These treatments may be applied as necessary and/or as indicated and may occur before, concurrent with or after administration of the solid dosage form described herein.

The dosage regimen can be any of a variety of methods and amounts, and can be determined by one skilled in the art according to known clinical factors. As is known in the medical arts, dosages for any one patient can depend on many factors, including the subject’s species, size, body surface area, age, sex, immunocompetence, and general health, the particular microorganism to be administered, duration and route of administration, the kind and stage of the disease, for example, tumor size, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a pharmaceutical agent (e.g., in a solid dosage form) described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., autoimmune disease, inflammatory disease, metabolic disease, dysbiosis, or cancer), delay its onset, or slow or stop its progression, or relieve one or more symptoms of the disease. One skilled in the art will recognize that dosage will depend upon a variety of factors including the strength of the particular agent (e.g., pharmaceutical agent) employed, as well as the age, species, condition, and body weight of the subject. The size of the dose will also be determined by the route, timing, and frequency of administration as well as the existence, nature, and extent of any adverse side-effects that might accompany the administration of a particular pharmaceutical agent and the desired physiological effect.

Suitable doses and dosage regimens can be determined by conventional range-finding techniques known to those of ordinary skill in the art. Generally, treatment is initiated with smaller dosages, which are no more than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the pharmaceutical agents used in accordance with the invention vary depending on the active agent, the age, weight, and clinical condition of the recipient patient, and the experience and judgment of the clinician or practitioner administering the therapy, among other factors affecting the selected dosage. For example, for cancer treatment, the dose should be sufficient to result in slowing, and preferably regressing, the growth of a tumor and most preferably causing complete regression of the cancer, or reduction in the size or number of metastases As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a solid dosage form, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be no more than the time period for a subject to mount an immune response, such as no more than about one week, no more than about ten days, no more than about two weeks, or no more than about a month.

In some embodiments, the delivery of an additional therapeutic agent in combination with the solid dosage form described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent which is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylasix, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-planter erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertisio, water retention, weakness, weight loss, weight gain, and xerostomia. In general, toxicity is acceptable if the benefits to the subject achieved through the therapy outweigh the adverse events experienced by the subject due to the therapy.

### Immune Disorders

In some embodiments, the methods and solid dosage forms described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn’s disease or ulcerative colitis). In some embodiments, the disease or disorder is psoriasis. In some embodiments, the disease or disorder is atopic dermatitis.

The methods and solid dosage forms described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring a such a disease or disorder.

The solid dosage forms described herein can be used, for example, as a pharmaceutical composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto’s disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of immune cells.

In some embodiments, the methods and solid dosage forms provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget’s disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and solid dosage forms described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and solid dosage forms described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn’s disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet’s disease, sarcoidosis, scleroderma, IBD-associated dysplasia, dysplasia associated masses or lesions, and primary sclerosing cholangitis.

Examples of reproductive system immune disorders which may be treated with the methods and solid dosage forms described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and solid dosage forms described herein may be used to treat autoimmune conditions having an inflammatory component. Such conditions include, but are not limited to, acute disseminated alopecia universalise, Behcet’s disease, Chagas’ disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn’s disease, diabetes mellitus type 1, giant cell arteritis, goodpasture’s syndrome, Grave’s disease, Guillain-Barre syndrome, Hashimoto’s disease, Henoch-Schonlein purpura, Kawasaki’s disease, lupus erythematosus, microscopic colitis, microscopic polyarteritis, mixed connective tissue disease, Muckle-Wells syndrome, multiple sclerosis, myasthenia gravis, opsoclous myoclonus syndrome, optic neuritis, ord’s thyroiditis, pemphigus, polyarteritis nodosa, polymyalgia, rheumatoid arthritis, Reiter’s syndrome, Sjogren’s syndrome, temporal arteritis, Wegener’s granulomatosis, warm autoimmune haemolytic anemia, interstitial cystitis, Lyme disease, morphea, psoriasis, sarcoidosis, scleroderma, ulcerative colitis, and vitiligo.

The methods and solid dosage forms described herein may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and solid dosage forms include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary’s syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Metabolic Disorders

In some embodiments, the methods and solid dosage forms described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and pharmaceutical compositions described herein relate to the treatment of Nonalcoholic Fatty Liver Disease (NAFLD) and Nonalcoholic Steatohepatitis (NASH).

The methods and solid dosage forms described herein can be used to treat any subject in need thereof. As used herein, a “subject in need thereof includes any subject that has a metabolic disease or disorder, as well as any subject with an increased likelihood of acquiring a such a disease or disorder.

The solid dosage forms described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH), or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Cancer

In some embodiments, the methods and solid dosage forms described herein relate to the treatment of cancer. In some embodiments, any cancer can be treated using the methods described herein. Examples of cancers that may treated by methods and solid dosage forms described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma, small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinonia, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget’s disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma, leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma, fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi’s sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing’s sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin’s disease; Hodgkin’s lymphoma; paragranuloma; malignant lymphoma, small lymphocytic; malignant lymphoma, large cell, diffuse; malignant lymphoma, follicular; mycosis fungoides; other specified non-Hodgkin’s lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises breast cancer (e.g., triple negative breast cancer).

In some embodiments, the cancer comprises colorectal cancer (e.g., microsatellite stable (MSS) colorectal cancer).

In some embodiments, the cancer comprises renal cell carcinoma.

In some embodiments, the cancer comprises lung cancer (e.g., non small cell lung cancer).

In some embodiments, the cancer comprises bladder cancer.

In some embodiments, the cancer comprises gastroesophageal cancer.

In some embodiments, the methods and solid dosage forms provided herein relate to the treatment of a leukemia. The term “leukemia” includes broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross’ leukemia, Rieder cell leukemia, Schilling’s leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and solid dosage forms provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher’s carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and solid dosage forms provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing′ s sarcoma, fascial sarcoma, fibroblastic sarcoma, giant cell sarcoma, Abernethy’s sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms’ tumor sarcoma, granulocytic sarcoma, Hodgkin’s sarcoma, idiopathic multiple pigmented hemorrhagic sarcoma, immunoblastic sarcoma of B cells, lymphoma, immunoblastic sarcoma of T-cells, Jensen’s sarcoma, Kaposi’s sarcoma, Kupffer cell sarcoma, angiosarcoma, leukosarcoina, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and solid dosage forms described herein include Hodgkin’s Disease, Non-Hodgkin’s Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman’s melanoma, S91 melanoma, nodular melanoma subungal melanoma, and superficial spreading melanoma.

Particular categories of tumors that can be treated using methods and solid dosage forms described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing’s tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms’ tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin′ s lymphoma, non-Hodgkin’s lymphoma, plasmacytoma, colorectal cancer, and rectal cancer.

Cancers treated in certain embodiments also include precancerous lesions, e.g., actinic keratosis (solar keratosis), moles (dysplastic nevi), acitinic chelitis (farmer’s lip), cutaneous horns, Barrett’s esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

### Other Diseases and Disorders

In some embodiments, the methods and solid dosage forms described herein relate to the treatment of liver diseases. Such diseases include, but are not limited to, Alagille Syndrome, Alcohol-Related Liver Disease, Alpha-1 Antitrypsin Deficiency, Autoimmune Hepatitis, Benign Liver Tumors, Biliary Atresia, Cirrhosis, Galactosemia, Gilbert Syndrome, Hemochromatosis, Hepatitis A, Hepatitis B, Hepatitis C, Hepatic Encephalopathy, Intrahepatic Cholestasis of Pregnancy (ICP), Lysosomal Acid Lipase Deficiency (LAL-D), Liver Cysts, Liver Cancer, Newborn Jaundice, Primary Biliary Cholangitis (PBC), Primary Sclerosing Cholangitis (PSC), Reye Syndrome, Type I Glycogen Storage Disease, and Wilson Disease.

The methods and solid dosage forms described herein may be used to treat neurodegenerative and neurological diseases. In certain embodiments, the neurodegenerative and/or neurological disease is Parkinson’s disease, Alzheimer’s disease, prion disease, Huntington’s disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Dysbiosis

In recent years, it has become increasingly clear that the gut microbiome (also called the “gut microbiota”) can have a significant impact on an individual’s health through microbial activity and influence (local and/or distal) on immune and other cells of the host (Walker, W.A., Dysbiosis. The Microbiota in Gastrointestinal Pathophysiology. Chapter 25. 2017; Weiss and Thierry, Mechanisms and consequences of intestinal dysbiosis. Cellular and Molecular Life Sciences. (2017) 74(16):2959-2977. Zurich Open Repository and Archive, doi: https://doi.org/10.1007/s00018-017-2509-x)).

A healthy host-gut microbiome homeostasis is sometimes referred to as a “eubiosis” or “normobiosis,” whereas a detrimental change in the host microbiome composition and/or its diversity can lead to an unhealthy imbalance in the microbiome, or a “dysbiosis” (Hooks and O′Malley. Dysbiosis and its discontents. American Society for Microbiology. October 2017. Vol. 8. Issue 5. mBio 8:e01492-17. https://doi.org/10.1128/mBio.01492-17). Dysbiosis, and associated local or distal host inflammatory or immune effects, may occur where microbiome homeostasis is lost or diminished, resulting in: increased susceptibility to pathogens; altered host bacterial metabolic activity; induction of host proinflammatory activity and/or reduction of host anti-inflammatory activity. Such effects are mediated in part by interactions between host immune cells (e.g., T cells, dendritic cells, mast cells, NK cells, intestinal epithelial lymphocytes (IEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359: 1098-99 (2018); Srinivasan et al., TEER measurement techniques for in vitro barrier model systems. J. Lab. Autom. 20: 107-126 (2015). A gastrointestinal dysbiosis can have physiological and immune effects within and outside the gastrointestinal tract.

The presence of a dysbiosis has been associated with a wide variety of diseases and conditions including: infection, cancer, autoimmune disorders (e.g., systemic lupus erythematosus (SLE)) or inflammatory disorders (e.g., functional gastrointestinal disorders such as inflammatory bowel disease (IBD), ulcerative colitis, and Crohn’s disease), neuroinflammatory diseases (e.g., multiple sclerosis), transplant disorders (e.g., graft-versus-host disease), fatty liver disease, type I diabetes, rheumatoid arthritis, Sjögren’s syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med .375:2369-79 (2016), Carding et al., Dysbiosis of the gut microbiota in disease. Microb. Ecol. Health Dis. (2015); 26: 10: 3402/mehd.v26.2619; Levy et al, Dysbiosis and the Immune System, Nature Reviews Immunology 17:219 (April 2017).

Exemplary pharmaceutical compositions and/or solid dosage forms disclosed herein can treat a dysbiosis and its effects by modifying the immune activity present at the site of dysbiosis. As described herein, such compositions can modify a dysbiosis via effects on host immune cells, resulting in, e.g., an increase in secretion of anti-inflammatory cytokines and/or a decrease in secretion of pro-inflammatory cytokines, reducing inflammation in the subject recipient or via changes in metabolite production.

Exemplary pharmaceutical compositions and/or solid dosage forms disclosed herein that are useful for treatment of disorders associated with a dysbiosis contain one or more types of immunomodulatory bacteria (e.g., anti-inflammatory bacteria) and/or mEVs (microbial extracellular vesicles) derived from such bacteria. Such compositions are capable of affecting the recipient host’s immune function, in the gastrointestinal tract, and/or a systemic effect at distal sites outside the subject’s gastrointestinal tract.

Exemplary pharmaceutical compositions and/or solid dosage forms disclosed herein that are useful for treatment of disorders associated with a dysbiosis contain a population of immunomodulatory bacteria of a single bacterial species (e.g., a single strain) (e.g., anti-inflammatory bacteria) and/or mEVs derived from such bacteria. Such compositions are capable of affecting the recipient host’s immune function, in the gastrointestinal tract, and /or a systemic effect at distal sites outside the subject’s gastrointestinal tract.

In one embodiment, pharmaceutical compositions and/or solid dosage forms containing an isolated population of immunomodulatory bacteria (e.g., anti-inflammatory bacterial cells) and/or mEVs derived from such bacteria are administered (e.g., orally) to a mammalian recipient in an amount effective to treat a dysbiosis and one or more of its effects in the recipient. The dysbiosis may be a gastrointestinal tract dysbiosis or a distal dysbiosis.

In another embodiment, pharmaceutical compositions and/or solid dosage forms of the instant invention can treat a gastrointestinal dysbiosis and one or more of its effects on host immune cells, resulting in an increase in secretion of anti-inflammatory cytokines and/or a decrease in secretion of pro-inflammatory cytokines, reducing inflammation in the subject recipient.

In another embodiment, the pharmaceutical compositions and/or solid dosage forms can treat a gastrointestinal dysbiosis and one or more of its effects by modulating the recipient immune response via cellular and cytokine modulation to reduce gut permeability by increasing the integrity of the intestinal epithelial barrier.

In another embodiment, the pharmaceutical compositions and/or solid dosage forms can treat a distal dysbiosis and one or more of its effects by modulating the recipient immune response at the site of dysbiosis via modulation of host immune cells.

Other exemplary pharmaceutical compositions and/or solid dosage forms are useful for treatment of disorders associated with a dysbiosis, which compositions contain one or more types of bacteria and/or mEVs capable of altering the relative proportions of host immune cell subpopulations, e.g., subpopulations of T cells, immune lymphoid cells, dendritic cells, NK cells and other immune cells, or the function thereof, in the recipient.

Other exemplary pharmaceutical compositions and/or solid dosage forms are useful for treatment of disorders associated with a dysbiosis, which compositions contain a population of immunomodulatory bacteria and/or mEVs of a single bacterial species e.g., a single strain) capable of altering the relative proportions of immune cell subpopulations, e.g., T cell subpopulations, immune lymphoid cells, NK cells and other immune cells, or the function thereof, in the recipient subject.

In one embodiment, the invention provides methods of treating a gastrointestinal dysbiosis and one or more of its effects by orally administering to a subject in need thereof a pharmaceutical composition and/or solid dosage forms which alters the microbiome population existing at the site of the dysbiosis. The pharmaceutical composition and/or solid dosage form can contain one or more types of immunomodulatory bacteria and/or mEVs or a population of immunomodulatory bacteria or mEVs of a single bacterial species (e.g., a single strain).

In one embodiment, the invention provides methods of treating a distal dysbiosis and one or more of its effects by orally administering to a subject in need thereof a pharmaceutical composition and/or solid dosage form which alters the subject’s immune response outside the gastrointestinal tract. The pharmaceutical composition and/or solid dosage form can contain one or more types of immunomodulatory bacteria and/or mEVs or a population of immunomodulatory bacteria or mEVs of a single bacterial species (e.g., a single strain).

In exemplary embodiments, pharmaceutical compositions and/or solid dosage forms useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, pharmaceutical compositions and/or solid dosage forms useful for treatment of disorders associated with a dysbiosis that decrease (e.g., inhibit) secretion of one or more pro-inflammatory cytokines by host immune cells. Pro-inflammatory cytokines include, but are not limited to, IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (e.g., orally administering) to the subject a therapeutic composition in the form of a probiotic or medical food comprising bacteria and/or mEVs in an amount sufficient to alter the microbiome at a site of the dysbiosis, such that the disorder associated with the dysbiosis is treated.

In another embodiment, a therapeutic composition of the instant invention in the form of a probiotic or medical food may be used to prevent or delay the onset of a dysbiosis in a subject at risk for developing a dysbiosis.

### Methods of Making Enhanced Bacteria

In certain aspects, provided herein are methods of making engineered bacteria for the production of the bacteria and/or mEVs (such as smEVs and/or pmEVs) described herein. In some embodiments, the engineered bacteria are modified to enhance certain desirable properties. For example, in some embodiments, the engineered bacteria are modified to enhance the immunomodulatory and/or therapeutic effect of the bacteria and/or mEVs (such as smEVs and/or pmEVs) (e.g., either alone or in combination with another therapeutic agent), to reduce toxicity and/or to improve bacterial and/or mEV (such as smEV and/or pmEV) manufacturing (e.g., higher oxygen tolerance, improved freeze-thaw tolerance, shorter generation times). The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, CRISPR/Cas9, or any combination thereof

In some embodiments of the methods provided herein, the bacterium is modified by directed evolution. In some embodiments, the directed evolution comprises exposure of the bacterium to an environmental condition and selection of bacterium with improved survival and/or growth under the environmental condition. In some embodiments, the method comprises a screen of mutagenized bacteria using an assay that identifies enhanced bacterium. In some embodiments, the method further comprises mutagenizing the bacteria (e.g., by exposure to chemical mutagens and/or UV radiation) or exposing them to a therapeutic agent (e.g., antibiotic) followed by an assay to detect bacteria having the desired phenotype (e.g., an in vivo assay, an ex vivo assay, or an in vitro assay).

### Gamma- Irradiation: Sample Protocol

Powders are gamma-irradiated at 17.5 kGy radiation unit at ambient temperature. Frozen biomasses are gamma-irradiated at 25 kGy radiation unit in the presence of dry ice.

### Frozen Biomass Preparation: Sample Protocol

After a desired level of bacterial culture growth is achieved, centrifuge cultures, discard the supernatant, leaving the pellet as dry as possible. Vortex the pellet to loosen the biomass. Resuspend pellet in desired cryoprotectant solution, transfer to cryogenic tube and snap freeze in liquid nitrogen. Store in -80 degree C freezer.

### Powder Preparation: Sample Protocol

After desired level of bacterial culture growth is achieved, centrifuge cultures, discard the supernatant, leaving the pellet as dry as possible. Resuspend pellet in desired cryoprotectant solution to create a formulated cell paste. The cryoprotectant may contain, e.g., maltodextrin, sodium ascorbate, sodium glutamate, and/or calcium chloride. Load the formulated cell paste onto stainless steel trays and load into a freeze drier, e.g., operating in automated mode with defined cycle parameters. The freeze dried product is fed into a milling machine and the resulting powder is collected.

Powders are stored (e.g., in vacuum sealed bags) at 2-8° C. (e.g., at 4° C.), e.g., in a desiccator.

After preparation and/or isolation, batches of mEVs are prepared as powders in similar fashion.

### EXAMPLES

### Example 1: Preparation of a Solid Dosage Form Comprising Extracellular Vesicles from Prevotella Histicola Strain B

The following recipe in Table 5 was prepared. The secreted microbial extracellular vesicles (smEVs) referred to below were from deposited Prevotella histicola Strain B 50329 (NRRL accession number B 50329). A disintegration study was performed to see how fast 1.5 mm minitablets of this recipe disintegrated. The resulting average disintegration times shown below in Table 5 are average times. DT in the table below stands for disintegration time in minutes: seconds.

TABLE 5

1.5 mm Mini-tablet

| Composition | % (w/w) | |
|---|---|---|
| Powder of smEVs from Prevotella histicola strain B 50329 | 25 | 5 |
| Mannitol (SD 200) | 61 | 80.5 |
| LHPC LH-11 | 5 | 5 |
| Crospovidone CL-F | 7 | 7 |
| Magnesium stearate | 1.5 | 2 |
| Colloidal silicon dioxide | 0.5 | 0.5 |
| Ave. DT (MM:SS) | 2:52 | 0:11 |

### Example 2: Preparation of a Solid Dosage Form Comprising Prevotella Histicola Strain B

The following recipes in Table 6 were prepared. The Prevotella histicola Strain B 50329 strain referred to below has been deposited as Prevotella histicola Strain B 50329 (NRRL accession number B 50329). A disintegration study was performed to see how fast 1.5 mm, 2 mm, or 3 mm minitablets of this recipe disintegrated. The resulting average disintegration times shown below in Table 6 are average times. DT in the table below stands for disintegration time in minutes: seconds.

TABLE 6

| | 1.5 mm Mini-tablet | | | 2 mm Mini-tablet | 3 mm Mini-tablet | | |
|---|---|---|---|---|---|---|---|
| Composition | % (w/w) | | | %, (w/w) | % (w/w) | | |
| Prevotella histicola Strain B 50329 powder | 50 | 30 | 10 | 16 | 60 | 50 | 5 |
| Mannitol (SD 200) | 36.5 | 56.5 | 76 | 70.5 | 26.5 | 36.5 | 81.5 |
| LHPC LH-11 | 5 | 5 | 5 | 5 | 5 | 5 | 5 |
| Crospovidone CL-F | 7 | 7 | 7 | 7 | 7 | 7 | 7 |
| Magnesium stearate | 1 | 1 | 1.5 | 1 | 1 | 1 | 1 |
| Colloidal silicon dioxide | 0.5 | 0.5 | 0.5 | 0.5 | 0.5 | 0.5 | 0.5 |
| Ave. DT (MM:SS) | 2:07 | 1:12 | 0:12 | 1:02 | 3:50 | 3:15 | 0:09 |

### Example 3: Preparation of a Solid Dosage Form Comprising Veillonella Parvula

The following recipes in Table 7 were prepared. The Veillonella parvula strain referred to below has been deposited as Veillonella parvula (ATCC designation number PTA-125691). The Veillonella parvula in the powder were gamma irradiated. A disintegration study was performed to see how fast 1.5 mm and 2 mm minitablets of this recipe disintegrated. The resulting average disintegration times shown below in Table 7 are average times. DT in the table below stands for disintegration time in minutes: seconds.

TABLE 7

| | 1.5 mm Mini-tablet | | | | 2 mm Mini-tablet | | | |
|---|---|---|---|---|---|---|---|---|
| Composition | % (w/w) | | | | % (w/w) | | | |
| Veillonella parvula powder | 50 | 30 | 10 | 3 | 50 | 16 | 5 | 1.6 |
| Mannitol (SD 200) | 36.5 | 56.5 | 76 | 83 | 36.5 | 70.5 | 81.5 | 84.9 |
| LHPC LH-11 | 5 | 5 | 5 | 5 | 5 | 5 | 5 | 5 |
| Crospovidone CL-F | 7 | 7 | 7 | 7 | 7 | 7 | | 7 |
| Magnesium stearate | 1 | 1 | 1.5 | 1.5 | 1 | 1 | 1 | 1 |
| Colloidal silicon dioxide | 0.5 | 0.5 | 0.5 | 0.5 | 0.5 | 0.5 | 0.5 | 0.5 |
| Ave. DT (MM:SS) | 4:47 | 1:11 | 0:20 | 0:13 | 7:26 | 1:11 | 0:22 | 0:10 |

### Example 4: Preparation of a Solid Dosage Form Comprising Bifidobacterium Animalis Ssp. Lactis

The following recipes in Table 8 were prepared. The Bifidobacterium animalis ssp. lactis strain referred to below has been deposited as Bifidobacterium animalis ssp. lactis (ATCC designation number PTA-125097). A disintegration study was performed to see how fast 1.5 mm and 2 mm minitablets of this recipe disintegrated. The resulting average disintegration times shown below in Table 8 are average times. DT in the table below stands for disintegration time in minutes: seconds.

TABLE 8

| | 1.5 mm Mini-tablet | | | 2 mm Mini-tablet |
|---|---|---|---|---|
| Composition | % (w/w) | | | % (w/w) |
| Bifidobacterium animalis ssp. Lactis powder | 50 | 30 | 10 | 50 |
| Mannitol (SD 200) | 36.5 | 56.5 | 76 | 36.5 |
| LHPC LH-11 | 5 | 5 | 5 | 5 |
| Crospovidone CL-F | 7 | 7 | 7 | 7 |
| Magnesium stearate | 1 | 1 | 1.5 | 1 |
| Colloidal silicon dioxide | 0.5 | 0.5 | 0.5 | 0.5 |
| Ave. DT (MM:SS) | 2:16 | 2:2.1 | 0:16 | 6:14 |

### Example 5: Preparation of a Solid Dosage Form Comprising Lactococcus Lactis Cremoris

The following recipe in Table 9 was prepared. The Lactococcus lactis cremoris Strain A strain referred to below has been deposited as Lactococcus lactis cremoris Strain A (ATCC designation number PTA- 125368).

TABLE 9

3 mm Mini-tablet

| Composition | % (w/w) |
|---|---|
| Lactococcus lactis cremoris powder | 50 |
| Mannitol (SD 200) | 36.5 |
| LHPC LH-11 | 5 |
| Crospovidone CL-F | 7 |
| Magnesium stearate | 1 |
| Colloidal silicon dioxide | 0.5 |

### Example 6: Preparation of a of Solid Dosage Form Comprising Prevotella Histicola Strain B

The following recipes in Table 10 were prepared as 3 mm mini-tablets. The Prevotella histicola Strain B 50329 strain referred to below has been deposited as Prevotella histicola Strain B 50329 (NRRL accession number B 50329).

TABLE 10

| | Formulation 1 w/w (%) | | | Formulation 2 w/w (%) | | |
|---|---|---|---|---|---|---|
| Composition | | | | | | |
| Prevotella histicola Strain B 50329 powder | 50 | 60 | 6 | 25 | 60 | 5 |
| Mannitol 200 SD | 36 | 26 | 80 | 61 | 26 | 81 |
| Low-substituted hydroxypropyl cellulose (L-HPC, LH-11) | 5 | | | | | |
| Crospovidone (Kollidon CL-F) | 7 | | | | | |
| Magnesium stearate | 1.5 | | | | | |
| Colloidal silica dioxide | 0.5 | | | | | |

Powder characteristics for the Formulation 2 preparations are provided in Table

TABLE 11

| | | | |
|---|---|---|---|
| Formulation 2 | 5% | 25% | 60% |
| Powder Weight (g) | 14.666 | 15.686 | 11.052 |
| Bulk Volume V1 (mL) | 34 | 37 | 26. 5 |
| Tap Volume V2 (mL) | 26 | 2.8 | 20.2 |
| Bulk Density (g/mL) | 0.43 | 0.42 | 0.42 |
| Tap Density (g/mL) | 0.56 | 0.56 | 0.55 |
| Hausner Ratio (V1/V2) | 1.31 | 1.32 | 1.31 |
| Flow through Orifice 10 mm (g/s) | 3.2 | 3.2 | 3. 3 |
| Flow through Orifice 25 mm (g/s) | 14.7 | 8.3 | 12.3 |

### Example 7: Preparation of a Solid Dosage Form Comprising Extracellular Vesicles from Prevotella Histicola Strain B

The following recipes in Table 12 were prepared. The secreted microbial extracellular vesicles (smEVs) referred to below were from deposited Prevotella histicola Strain B 50329 (NRRL accession number B 50329).

TABLE 12

Active-0.5% low dose

| Composition | %w/w |
|---|---|
| Powder of smEVs from Prevotella histicola strain B 50329 | 0.50% |
| SiO2 | 1.00% |
| Mannitol | 90.5% |
| Crospovidone | 7.00% |
| MgSt | 1.00% |
| Total | 100.00% |
| Active-5% medium dose | |
| Composition | %w/w |
| Powder of smEVs from Prevotella histicola strain B 50329 | 5.00% |
| Mannitol | 86.00% |
| SiO2 | 1.00% |
| Crospovidone | 7.00% |
| MgSt | 1.00% |
| Total | 100.00% |
| Active-25% high dose | |
| Composition | %w/w |
| Powder of smEVs from Prevotella histicola strain B 50329 | 25.00% |
| Mannitol | 66.00% |
| SiO2 | 1.00% |
| Crospovidone | 7.00% |
| MgSt | 1.00% |
| Total | 100.00% |

### Example 8: In Vivo Efficacy of Solid Dosage Forms of Secreted Microbial Extracellular Vesicles From Prevotella Histicola Strain B

Solid dosage forms containing secreted microbial extracellular vesicles (smEVs) from Prevotella histicola Strain B (NRRL accession number B 50329) were tested in an in vivo DTH model of inflammation. The 1.5 mm minitablet recipes in Table 12 were used to prepare the 1.5 mm mini-mini-tabs (MMTs).

Female 8 week old C57BL/6 mice were purchased from Taconic Biosciences and acclimated at a vivarium for one week. Mice were primed with an emulsion of KLH and CFA (1: 1) by subcutaneous immunization on day 0. On each day of dosing (days 5-8), mice were anesthetized with isoflurane and orally gavaged with the smEVs, dosed intraperitoneally with dexamethasone at 1 mg/kg, or gavaged with uncoated 1.5 mm mini-mini-tabs (MMTs) according to the following protocol. A flexible rat gavage needle was attached to a syringe and 200 ul of PBS was drawn up, then an individual MMT was placed inside the bottom of the needle. The needle was placed into the esophagus of the anesthetized mouse and the plunger was injected quickly so the force would dislodge the MMT into the esophagus. A stainless steel tipped disposable mouse gavage needle was then used to tamp the MMT down to the stomach. After dosing on day 8, while mice were still anesthetized, left ears were measured for baseline measurements with Fowler calipers and the mice were challenged intradermally with KLH in saline (10 µl) in the left ear and ear thickness measurements were taken at 24 hours.

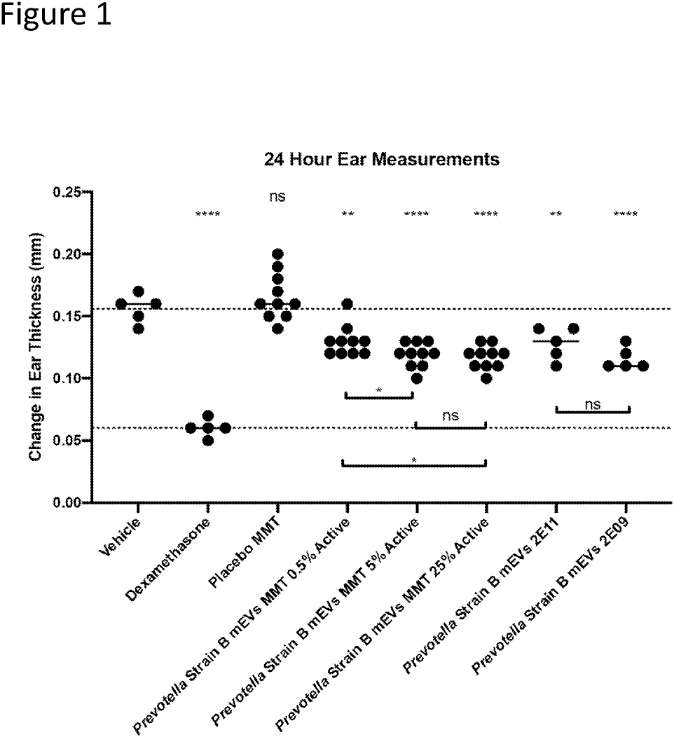

The 24-hour ear measurement results are shown in FIG. 1. MMTs made from Prevotella histicola Strain B 50329 mEVs as active ingredient show efficacy in a dose dependent manner (0.5% active (equivalent to 2.2E09 powder per tablet) shows a decrease in efficacy compared to 5% (equivalent to 2.2E10 powder per tablet) and 25% (equivalent to 1.1E+11 powder per tablet)). Similar efficacy was observed at equivalent MMT and smEV groups (Prevotella Strain B mEVs MMT 0.5% compared to Prevotella Strain B mEVs 2E+09 (liquid suspension), which saw a slight decrease in efficacy of the MMT, and Prevotella Strain B mEVs MMT 25% compared to Prevotella Strain B mEVs 2E+11 (liquid suspension), which showed no significant difference).

### Example 9: In Vivo Efficacy of Solid Dosage Forms of Prevotella HisticolaStrain B

Solid dosage forms containing Prevotella histicola Strain B (NRRL accession number B 50329) were tested in an in vivo DTH model of inflammation. The 1.5 mm minitablet recipes in Tablet 6 were used to prepare the 1.5 mm mini-mini-tabs (MMTs).

Female 8 week old C57BL/6 mice were purchased from Taconic Biosciences and acclimated at a vivarium for one week. Mice were primed with an emulsion of KLH and CFA (1: 1) by subcutaneous immunization on day 0. On each day of dosing (days 5-8), mice were anesthetized with isoflurane and orally gavaged with Prevotella histicola Strain B, dosed intraperitoneally with dexamethasone at 1 mg/kg, or gavaged with uncoated 1.5 mm mini-mini-tabs (MMTs) according to the following protocol. A flexible rat gavage needle was attached to a syringe and 200 ul of PBS was drawn up, then an individual MMT was placed inside the bottom of the needle. The needle was placed into the esophagus of the anesthetized mouse and the plunger was injected quickly so the force would dislodge the MMT into the esophagus. A stainless steel tipped disposable mouse gavage needle was then used to tamp the MMT down to the stomach. After dosing on day 8, while mice were still anesthetized, left ears were measured for baseline measurements with Fowler calipers and the mice were challenged intradermally with KLH in saline (10 µl) in the left ear and ear thickness measurements were taken at 24 hours.

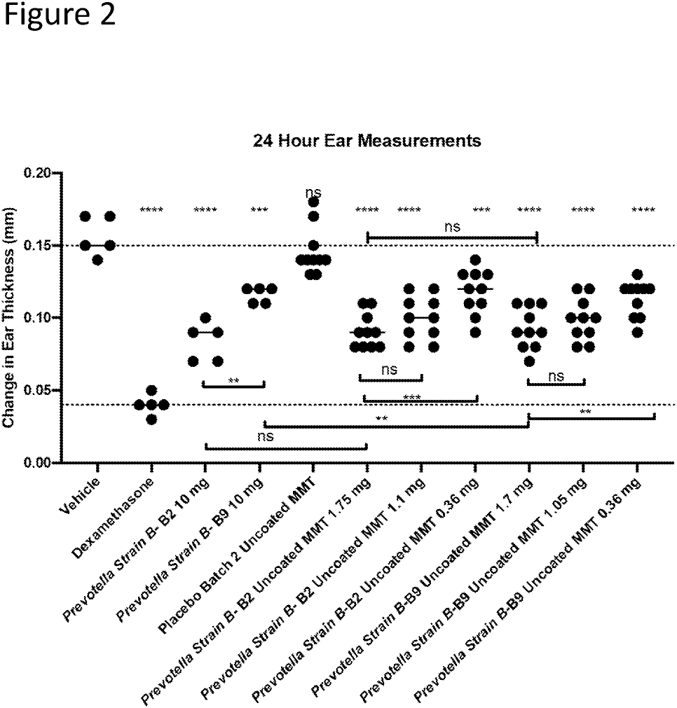

The 24-hour ear measurement results are shown in FIG. 2, MMTs made from Prevotella histicola Strain B 50329 from two different process batches (B2 and B9) were compared at three doses (1.75/1.7 mg, 1.1/1.05 mg and 0.36 mg) and against powder from each batch at 10 mg. In this study, MMTs made from 1.7 mg of powder are equally or more efficacious than powder alone at 10 mg. There was no significant difference between the MMTs made from B2 and B9.

### Example 10:In Vivo Efficacy of Solid Dosage Forms of Veillonella Parvula

Solid dosage forms containing gamma irradiated (GI) Veillonella parvula (ATCC designation number PTA-125691) were tested in an in vivo DTH model of inflammation. The 1.5 mm minitablet recipes in Tablet 7 were used to prepare the 1.5 mm mini-mini-tabs (MMTs).

Female 8 week old C57BL/6 mice were purchased from Taconic Biosciences and acclimated at a vivarium for one week. Mice were primed with an emulsion of KLH and CFA (1:1) by subcutaneous immunization on day 0. On each day of dosing (days 5-8), mice were anesthetized with isoflurane and orally gavaged with Veillonella parvula, dosed intraperitoneally with dexamethasone at 1 mg/kg, or gavaged with uncoated 1.5 mm mini-mini-tabs (MMTs) according to the following protocol. A flexible rat gavage needle was attached to a syringe and 200 ul of PBS was drawn up, then an individual MMT was placed inside the bottom of the needle. The needle was placed into the esophagus of the anesthetized mouse and the plunger was injected quickly so the force would dislodge the MMT into the esophagus. A stainless steel tipped disposable mouse gavage needle was then used to tamp the MMT down to the stomach. After dosing on day 8, while mice were still anesthetized, left ears were measured for baseline measurements with Fowler calipers and the mice were challenged intradermally with KLH in saline (10 µl) in the left ear and ear thickness measurements were taken at 24 hours.

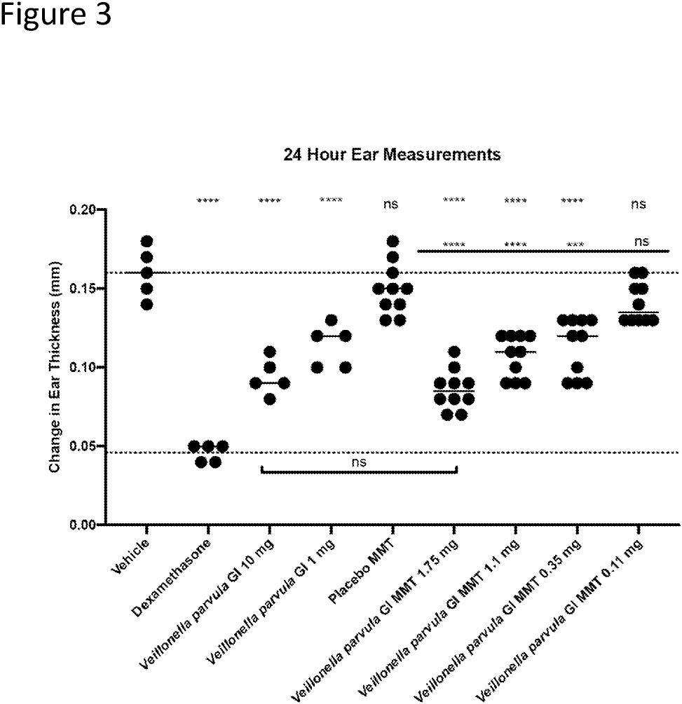

The 24 hour ear measurement results are shown in FIG. 3. MMTs made from Veillonella parvula were tested in a dose response at 1.75 mg, 1.1 mg, 0.35 mg and 0.11 mg and against powder alone at 10 mg and 1 mg. The MMTs at the three highest doses were efficacious, however the lowest dose, 0.11 mg lost efficacy. There was a dose response trend seen for the MMTs. The highest dose of MMTs (1.75 mg) was equally as efficacious as the 10 mg powder dose.

### Example 10: Representative Strains As Sources for EVs

Secreted microbial extracellular vesicles (smEVs) were isolated from the strains listed in Table J. Information on the Gram staining, cell wall structure, and taxonomic classification for each strain is also provided in Table J.

Bacteria of the taxonomic groups listed in Table J (e.g., class, order, family, genus, species or strain) can be used in the solid dosage forms described herein.

mEVs of bacteria of the taxonomic groups listed in Table 1 (e.g., class, order, family, genus, species or strain) can be used in the solid dosage forms described herein.

TABLE J

Strains from which extracellular vesicles (EVs) were isolated

| | | | | | | |
|---|---|---|---|---|---|---|
| Strain | Gram-stain | Cell envelope structure | Phylum | Class | Order | Family |
| Parabacteroides distasonis DRLU022118 A lLEUM-6 | Gram-stain-negative | diderm | Bacteroidota | Bacteroidia | Bacteroidales | Porphyromon adaceae |
| Parabacteroides goldsteinii S4 | Gram.-stain-negative | diderm | Bacteroidota | Bacteroidia | Bacteroidales | Porphyromon adaceae |
| Prevotella histicola | Gram-stain-negative | diderm | Bacteroidota | Bacteroidia | Bacteroidales | Prevotellaceae |
| Prevotella histicola | Gram-stain-negative | diderm | Bacteroidota | Bacteroidia | Bacteroidales | Prevotellaceae |
| Fournierella massiliensis S10 G1Mucosa-297 | Gram-stain-negative | monoderm | Firmicutes | Clostridia | Eubacteriales | Oscillospiraceae (formely Ruminococcaceae) |
| Harrxflintia acetispora S4-M5 | Gram-stain-negative | monoderm | Firmicutes | Clostridia | Eubacteriales | Oscillospiraceae |
| Blautia massiliensis S1046-4A5 | Gram-stain-negative | monoderm | Firmicutes | Clostridia | Eubacteriales | Lachnospiraceae |
| Mediterranei bacterl[Rumi nococcus] gnavus S10 GIMucosa-412 | Gram-stain-negative | monoderm | Firmicutes | Clostridia | Eubacteriales | Lachnospiraceae |
| Clostridioides difficile S10 GImucosa-525 | Gram-stain-positive | monoderm | Firmicutes | Clostridia | Eubacteriales | Peptostreptococcaceae |
| Aminipila sp. S16-M4 | Gram-stain-positive | monoderm | Firmicutes | Clostridia | Eubacteriales | Closttidiales Family XIII/Incerlae sedis41/[Eubacteriales, no family] |
| Megasphaera sp. S29-N3 | Gram-stain-negative | diderm | Firmicutes | Negativicutes | Veillonellales | Veillonellaceae |
| Megasphaera sp. S1007 | Gram-stain-negative | diderm | Firmicutes | Negativicutes | Veillonellales | Veillonellaceae |
| Selenomonas felix S34N-300R | Gram-stain-negative | diderm | Firmicutes | Negativicutes | Selenomonadales | Selenomonadaceae |
| Veillonella parvula | Gram-stain-negative | diderm | Firmicutes | Negativicutes | Veillonellales | Veillonellaceae |
| Propionispora sp. DSM100705-1A | Gram-stain-negative | diderm | Finnicutes | Negativicutes | Selenomonad ales | Sporomusaceae |
| Rarimicrobium hominis S24RS2-T2-5 | Gram-stain-negative | diderm | Synergistota | Synergistia | Synergistales | Synergistaceae |
| Cloacibacillus evryensis S29-M8 | Gram-stain-negative | diderm | Synergistota | Synergistia | Synergistales | Synergistaceae |
| Veillonella parvula S14-205 | Gram-stain-negative | diderm | Firmicutes | Negativicutes | Veillonellales | Veillonellaceae |
| Veillonella sp/dispar ECD01-DP-201 | Gram-stain-negative | diderm | Fimricutes | Negativicutes | Veillonellales | Veillonellaceae |
| Veillonella parvula/dispar ECD01-DP-223 | Gram-stain-negative | diderm | Firmicules | Negativictiles | Veillonellales | Veillonellaceae |
| Veillonella parvula S16 GIMucosa-95 | Gram-stain-negative | diderm | Firmicutes | Negativicutes | Veillonellales | Veillonellaceae |

### Incorporation by Reference

All publications patent applications mentioned herein are hereby incorporated by reference in their entirety as if each individual publication or patent application was specifically and individually indicated to be incorporated by reference. In case of conflict, the present application, including any definitions herein, will control.

### Equivalents

Those skilled in the art will recognize or be able to ascertain using no more than routine experimentation, many equivalents to the specific embodiments of the invention described herein. Such equivalents are intended to be encompassed by the following claims.

What is claimed is:

1. A solid dosage form of a pharmaceutical composition comprising a pharmaceutical agent and one or more disintegrating agents, wherein the total mass of the one or more disintegrating agents is at least 5% of the total mass of the pharmaceutical composition and wherein the pharmaceutical agent comprises bacteria and/or microbial extracellular vesicles (mEVs).

2. The solid dosage form of claim 1, wherein the one or more disintegrating agents comprises L-HPC.

3. The solid dosage form of claim 2, wherein the L-HPC is L-HPC of grade LH-11.

4. The solid dosage form of claim 2 or 3, wherein the total L-HPC mass is at least 0.1% and no more than 10% of the total mass of the pharmaceutical composition.

5. The solid dosage form of any one of claims 1 to 4, wherein the one or more disintegrating agents comprises crospovidone.

6. The solid dosage form of claim 5, wherein the total crospovidone mass is at least 1% and no more than 15% of the total mass of the pharmaceutical composition.

7. The solid dosage form of any one of claims 1 to 6, wherein the total pharmaceutical agent mass is at least 0.5% and no more than 75% of the total mass of the pharmaceutical composition.

8. A solid dosage form of a pharmaceutical composition comprising:
a pharmaceutical agent having a total pharmaceutical agent mass that is at least 0.5% and no more than 75% of the total mass of the pharmaceutical composition, wherein the pharmaceutical agent comprises bacteria and/or microbial extracellular vesicles (mEVs); lowsubstituted hydroxypropyl cellulose (L-HPC) having a total L-HPC mass that is at least 0.1% and no more than 10% of the total mass of the pharmaceutical composition;
and crospovidone having a total crospovidone mass that is at least 1% and no more than 15% of the total mass of the pharmaceutical composition.

9. The solid dosage form of claim 8, wherein the total L-HPC mass plus the total crospovidone mass is at least 5% of the total mass of the pharmaceutical composition.

10. The solid dosage form of claim 8, wherein the total L-HPC mass plus the total crospovidone mass is at least 10% of the total mass of the pharmaceutical composition.

11. The solid dosage form of any one of claims 8 to 10, wherein the L-HPC is L-HPC of grade LH-11.

12. The solid dosage form of any one of claims 8 to 11, wherein the total L-HPC mass is at least 0.3% and no more than 7% of the total mass of the pharmaceutical composition; the total crospovidone mass is at least 5% and no more than 10% of the total mass of the pharmaceutical composition.

13. The solid dosage form of any one of claims 8 to 12, wherein the total L-HPC mass is at least 0.4% and no more than 6% of the total mass of the pharmaceutical composition; the total crospovidone mass is at least 6% and no more than 8% of the total mass of the pharmaceutical composition.

14. The solid dosage form of any one of claims 8 to 13, wherein the total L-HPC mass is at least 0.5% and no more than 5% of the total mass of the pharmaceutical composition; the total crospovidone mass is about 7% of the total mass of the pharmaceutical composition.

15. The solid dosage form of any one of claims 1 to 13, wherein the total pharmaceutical agent mass is at least 1.2% and no more than 75% of the total mass of the pharmaceutical composition.

16. The solid dosage form of any one of claims 1 to 13, wherein the total pharmaceutical agent mass is at least 1.4% and no more than 65% of the total mass of the pharmaceutical composition.

17. The solid dosage form of any one of claims 1 to 13, wherein the total pharmaceutical agent mass is at least 1.5% and no more than 63% of the total mass of the pharmaceutical composition.

18. The solid dosage form of any one of claims 1 to 13, wherein the total pharmaceutical agent mass is at least 1.6% and no more than 60% of the total mass of the pharmaceutical composition.

19. The solid dosage form of any one of claims 1 to 18, further comprising mannitol having a total mannitol mass that is at least 25% and no more than 95% of the total mass of the pharmaceutical composition.

20. The solid dosage form of any one of claims 1 to 19, further comprising magnesium stearate having a total magnesium stearate mass that is at least 0.01% and no more than 10% of the total mass of the pharmaceutical composition.

21. The solid dosage form of any one of claims 1 to 20, further comprising colloidal silicon dioxide having a total colloidal silicon dioxide mass that is at least 0.01% and no more than 10% of the total mass of the pharmaceutical composition.

22. The solid dosage form of claim 21, wherein the total pharmaceutical agent mass is at least 5% and no more than 25% of the total mass of the pharmaceutical composition; the total mannitol mass is at least 61% and no more than 80.5% of the total mass of the pharmaceutical composition; the total L-HPC mass is about 5% of the total mass of the pharmaceutical composition; the total crospovidone mass is about 7% of the total mass of the pharmaceutical composition; the total magnesium stearate mass is at least 1.5% and no more than 2% of the total mass of the pharmaceutical composition; and the total colloidal silicon dioxide mass is about 0.5% of the total mass of the pharmaceutical composition.

23. The solid dosage form of claim 21, wherein the total pharmaceutical agent mass is at least 5% and no more than 60% of the total mass of the pharmaceutical composition; the total mannitol mass is at least 26.5% and no more than 81.5% of the total mass of the pharmaceutical composition; the total L-HPC mass is about 5% of the total mass of the pharmaceutical composition; the total crospovidone mass is 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

24. The solid dosage form of claim 21, wherein the total pharmaceutical agent mass is at least 3% and no more than 50% of the total mass of the pharmaceutical composition; the total mannitol mass is at least 36.5% and no more than 84.9% of the total mass of the pharmaceutical composition; the total L-HPC mass is about 5% of the total mass of the pharmaceutical composition; the total crospovidone mass is 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

25. The solid dosage form of claim 21, wherein the total pharmaceutical agent mass is at least 10% and no more than 50% of the total mass of the pharmaceutical composition; the total mannitol mass is at least 56.5% and no more than 76% of the total mass of the pharmaceutical composition; the total L-HPC mass is about 5% of the total mass of the pharmaceutical composition; the total crospovidone mass is about 7% of the total mass of the pharmaceutical composition; the total magnesium stearate mass is at least 1% and no more than 1.5% of the total mass of the pharmaceutical composition; and the total colloidal silicon dioxide mass is about 0.5% of the total mass of the pharmaceutical composition.

26. The solid dosage form of claim 21, wherein the total pharmaceutical agent mass is about 50% of the total mass of the pharmaceutical composition; the total mannitol mass is about 36.5% of the total mass of the pharmaceutical composition; the total L-HPC mass is about 5% of the total mass of the pharmaceutical composition; the total crospovidone mass is about 7% of the total mass of the pharmaceutical composition; the total magnesium stearate mass is about 1% of the total mass of the pharmaceutical composition; and the total colloidal silicon dioxide mass is about 0.5% of the total mass of the pharmaceutical composition.

27. The solid dosage form of claim 21, wherein the total pharmaceutical agent mass is at least 5% and no more than 60% of the total mass of the pharmaceutical composition; the total mannitol mass is at least 26% and no more than 81% of the total mass of the pharmaceutical composition; the total L-HPC mass is about 5% of the total mass of the pharmaceutical composition; the total crospovidone mass is about 7% of the total mass of the pharmaceutical composition; the total magnesium stearate mass is about 1.5% of the total mass of the pharmaceutical composition; and the total colloidal silicon dioxide mass is about 0.5% of the total mass of the pharmaceutical composition.

28. The solid dosage form of claim 21, wherein the total pharmaceutical agent mass is about 0.5% of the total mass of the pharmaceutical composition; the total mannitol mass is about 90.5% of the total mass of the pharmaceutical composition; the total crospovidone mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

29. The solid dosage form of claim 21, wherein the total pharmaceutical agent mass is about 5% of the total mass of the pharmaceutical composition; the total mannitol mass is about 86% of the total mass of the pharmaceutical composition; the total crospovidone mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

30. The solid dosage form of claim 21, wherein the total pharmaceutical agent mass is about 25% of the total mass of the pharmaceutical composition; the total mannitol mass is about 66% of the total mass of the pharmaceutical composition; the total crospovidone mass is about 7% of the total mass of the pharmaceutical composition; the total magnesium stearate mass is about 1% of the total mass of the pharmaceutical composition; and the total colloidal silicon dioxide mass is about 1% of the total mass of the pharmaceutical composition.

31. The solid dosage form of any one of claims 1 to 30, wherein the pharmaceutical agent comprises bacteria.

32. The solid dosage form of claim 31, wherein the bacteria are lyophilized bacteria.

33. The solid dosage form of claim 31 or 32, wherein the bacteria are of the genus Lactococcus, Prevotella, Bifidobacterium, or Veillonella.

34. The solid dosage form of claim 31 or 32, wherein the bacteria are of the species Lactococcus lactis cremoris.

35. The solid dosage form of claim 34, wherein the Lactococcus lactis cremoris is Lactococcus lactis cremoris Strain A (ATCC designation number PTA-125368).

36. The solid dosage form of claim 31 or 32, wherein the bacteria are of the species Veillonella parvula.

37. The solid dosage form of claim 36, wherein the Veillonella parvula is Veillonella parvula (ATCC designation number PTA-125691).

38. The solid dosage form of claim 31 or 32, wherein the bacteria are of the species Prevotella histicola.

39. The solid dosage form of claim 38, wherein the Prevotella histicola is Prevotella histicola Strain B 50329 (NRRL, accession number B 50329).

40. The solid dosage form of claim 31 or 32, wherein the bacteria are of the species Bifidobacterium animalis.

41. The solid dosage form of claim 40, wherein the Bifidobacterium animalis is Bifidobacterium animalis ssp. lactis (ATCC designation number PTA-125097).

42. The solid dosage form of claim 31 or 32, wherein the bacteria are a species listed in Table 1, Table 2, or Table 3.

43. The solid dosage form of claim 31 or 32, wherein the bacteria are a bacterial strain that has at least 95% genomic, 16S ribosomal ribonucleic acid, or clustered regularly interspaced short palindromic repeats sequence identity with a strain listed in Table 1 or Table 3.

44. The solid dosage form of claim 31 or 32, wherein the bacteria are a bacterial strain that has at least 99% genomic, 16S ribosomal ribonucleic acid, or clustered regularly interspaced short palindromic repeats sequence identity with a strain listed in Table 1 or Table 3.

45. The solid dosage form of claim 31 or 32, wherein the bacteria are a bacterial strain listed in Table 1 or Table 3.

46. The solid dosage form of any one of claims 31 to 45, wherein the bacterial are live, attenuated, or dead.

47. The solid dosage form of any one of claims 1 to 30, wherein the pharmaceutical agent comprises mEVs.

48. The solid dosage form of claim 47, wherein the mEVs are isolated mEVs.

49. The solid dosage form of claim 47, wherein the mEVs are secreted mEVs.

50. The solid dosage form of claim 47, wherein the mEVs are processed mEVs.

51. The solid dosage form of any one of claims 1 to 50, wherein the solid dosage form is a minitablet.

52. The solid dosage form of claim 50, wherein the minitablet is a 1 mm minitablet, 1.5 mm minitablet, 2 mm minitablet, 3 mm minitablet, or 4 mm minitablet.

53. The solid dosage form of claim 51 or 52, wherein a plurality of minitablets are contained in a capsule.

54. The solid dosage form of any one of claims 1 to 53, further comprising an enteric coating.

55. The solid dosage form of claim 54, wherein the enteric coating is a single enteric coating or more than one enteric coating.

56. The solid dosage form of claim 54 or 55, wherein the enteric coating comprises an inner enteric coating and an outer enteric coating, and wherein the inner and outer enteric coatings are not identical.

57. The solid dosage form of claim any one of claims 54 to 56, wherein the enteric coating comprises a methacrylic acid ethyl acrylate (MAE) copolymer (1:1).

58. The solid dosage form of any one of claims 54 to 57, wherein the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

59. The solid dosage form of any one of claims 54 to 57, wherein the enteric coating comprises an anionic polymeric material.

60. A method of preventing or treating a disease of a subject, the method comprising administering to the subject a solid dosage form of any one of claims 1 to 59.

61. Use of a solid dosage form of any one of claims 1 to 59 for the treatment or prevention of a disease of a subject.

62. Use of a solid dosage form of any one of claims 1 to 59 for the preparation of a medicament for treating or preventing a disease in a subject.

63. A solid dosage form of any one of claims 1 to 59 for use in the treatment or prevention of disease of a subject.

64. A method of preparing a solid dosage form of a pharmaceutical composition, the method comprising:
(a) combining into a pharmaceutical composition:
(i) a pharmaceutical agent having a total pharmaceutical agent mass that is at least 1% and no more than 75% of the total mass of the pharmaceutical composition, wherein the pharmaceutical agent comprises bacteria and/or microbial extracellular vesicles (mEVs);
(ii) low-substituted hydroxypropyl cellulose (L-HPC) having a total L-HPC mass that is at least 0.1% and no more than 10% of the total mass of the pharmaceutical composition;
(iii) crospovidone having a total crospovidone mass that is at least 1% and no more than 15% of the total mass of the pharmaceutical composition; and
(b) compressing the pharmaceutical composition into a solid dosage form.

65. A method of preparing a solid dosage form of a pharmaceutical composition, the method comprising:
(a) combining into a pharmaceutical composition:
(i) a pharmaceutical agent having a total pharmaceutical agent mass that is at least 1% and no more than 75% of the total mass of the pharmaceutical composition, wherein the pharmaceutical agent comprises bacteria and/or microbial extracellular vesicles (mEVs);
(ii) crospovidone having a total crospovidone mass that is at least 1% and no more than 15% of the total mass of the pharmaceutical composition; and
(b) compressing the pharmaceutical composition into a solid dosage form.

66. The method of claim 64 or 65, further comprising the step of enterically coating the solid dosage form to obtain an enterically coated solid dosage form.

67. The method of any one of claims 64 to 66, wherein the solid dosage form is a minitablet.
